(19) United States
(12) Patent Application Publication
(10) Pub. No.: US 2011/0105472 A1
(43) Pub. Date: May 5, 2011

(54) Diaminopyrimidines as crop protection agents

(75) Inventors: Jörg Nico Greul, Leichlingen (DE); Olive Gaertzen, Koln (DE); Stefan Hillebrand, Neuss (DE); Amos Mattes, Langenfeld (DE); Ulrike Wachendorff-Neumann, Neuwied (DE); Peter Dahmen, Neuss (DE); Arnd Voerste, Koln (DE); Peter Schreier, Koln (DE); Ulrich Görgens, Ratingen (DE); Heinz Kehne, Hofheim (DE); Christian Paulitz, Liederbach (DE); Hiroyuki Hadano, Shimotsuke-shi (JP); Oliver Guth, Leverkusen (DE); Angela Becker, Dusseldorf (DE); Olga Malsam, Rosrath (DE)

(73) Assignee: Bayer CropScience AG, Monheim (DE)

(21) Appl. No.: 12/933,600

(22) PCT Filed: Mar. 16, 2009
(86) PCT No.: PCT/EP09/01902

§ 371 Date: Dec. 13, 2010

(30) Foreign Application Priority Data
Mar. 20, 2008 (EP) 08102819.3

(51) Int. Cl. A01N 43/54; C07D 403/12; C07D 411/12; C07D 401/12; C07D 405/12; C07D 413/12; A01N 43/84; A01P 3/00; A01P 17/00

(52) U.S. Cl. 514/212.07; 544/324; 540/523; 544/122; 540/524; 514/275; 514/230.8; 514/235.8; 514/212.08

(57) ABSTRACT

Use of diaminopyrimidines of the formula (I)

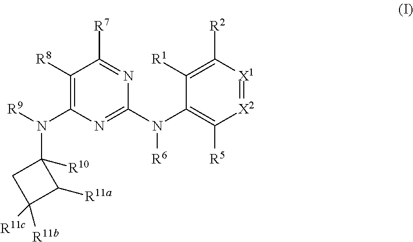

in which
R¹ to R^{11a,b,c} and X¹, X² have the meanings given in the description, and also agrochemically active salts thereof as crop protection agents.

Diaminopyrimidines of the formulae (Ia), (Ib) and (Ic)

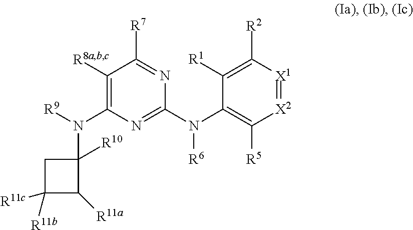

in which R^{8a}, R^{8b}, R^{8c}, and R¹, R², R³, R⁴, R⁵, R⁶, R⁷, R⁸, R⁹, R¹⁰, R^{11a,b,c} and X¹, X² have the meanings given in the description, and also agrochemically active salts thereof and their use for controlling animal pests and/or phytopathogenic harmful fungi.

The invention relates to cyclobutyl-substituted diaminopyrimidines and to their agrochemically active salts, to their use and also to methods and compositions for controlling animal pests and/or weeds and/or phytopathogenic harmful fungi in and/or on plants or in and/or on seed of plants, to processes for preparing such compositions and to treated seed and also to their use for controlling pests and/or weeds and/or phytopathogenic harmful fungi in agriculture, horticulture and forestry, in animal health, in the protection of materials and also in the domestic and hygiene field. The present invention furthermore relates to a process for preparing cyclobutyl-substituted diaminopyrimidines.

It is already known that certain alkynyl-substituted diaminopyrimidines can be employed as fungicidal crop protection agents (see DE 4029650 A1). However, in particular at low application rates, the fungicidal activity of these compounds is not always sufficient.

Since the ecological and economical demands made on modern crop protection agents are increasing constantly, for example with respect to activity spectrum, toxicity, selectivity, application rate, formation of residues and favourable manufacture, and there can furthermore be problems, for example, with resistance, there is a constant need to develop novel crop protection agents which, at least in some areas, help to overcome the disadvantages mentioned.

Surprisingly, it has now been found that the present cyclobutyl-substituted diaminopyrimidines achieve the objects mentioned at least in some aspects and are suitable as crop protection agents, in particular as fungicides, insecticides or herbicides.

Some of these cyclobutyl-substituted diaminopyrimidines are already known as pharmaceutically active compounds (see, for example, WO 2006/021544, WO 2007/072158, WO 2007/003596, WO 2005/016893, WO 05/013996, WO 04/056807, WO 04/014382, WO 03/030909), but not their surprising fungicidal, insecticidal or herbicidal activity.

The invention provides the use of compounds of the formula (I) as crop protection agents,

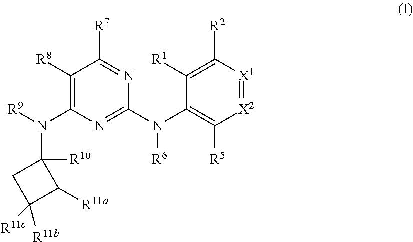

in which the symbols have the following meanings:
X¹ is nitrogen or CR³
X² is nitrogen or CR⁴
where X¹ and X² are not both nitrogen
R¹ and R⁵ independently of one another are hydrogen, C₁-C₄-alkyl, C₁-C₄-alkoxy or Hal
R² to R⁴ independently of one another are hydrogen, halogen, cyano, nitro, a 3- to 8-membered unsubstituted or substituted, saturated or unsaturated cycle which may contain no or up to four heteroatoms selected from the group consisting of N, O and S, where any two oxygen atoms are not adjacent to one another, OR¹², O(CH₂)ₘOR¹², O(CH₂)ₘN(R¹²)₂, O[C(R¹²)₂]ₘOR¹², O[C(R¹²)₂]ₘN(R¹²)₂, OSO₂N(R¹²)₂, OCON(R¹²)₂, OCOR¹³, SF₅, SR¹², SOR¹², SO₂R¹², SON(R¹²)₂, SO₂N(R¹²)₂, C═OR¹², CH═NOR¹², CR¹³═NOR¹², COCl, CON(R¹²)₂, COOR¹², COO(CH₂)ₘOR¹², CO(CH₂)ₘCN, NR¹²COR¹², NR¹²COOR¹³, NR¹²(C═S)OR¹³, N(R¹²)₂, NR¹²SO₂R¹³, NR¹²SOR¹³, C(R¹²)₂OR¹², [C(R¹²)₂]ₘCN, (CH₂)ₘC(R¹²)₂OR¹², (CH₂)ₘOR¹², (CH₂)ₘSR¹², [C(R¹²)₂]ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, [C(R¹²)₂]ₘN(R¹²)₂, (CH₂)ₘCOOR¹², (CH₂)ₘCOR¹², [C(R¹²)₂]ₘOR¹², [C(R¹²)₂]ₘCOR¹², [C(R¹²)₂]ₘCON(R¹²)₂, (CH₂)ₘNR¹²COR¹², (CH₂)ₘNR¹²COOR¹³, [C(R¹²)₂]ₘNR¹²COR¹², [C(R¹²)₂]ₘNR¹²COOR¹³, [C(R¹²)₂]ₘNR¹²OR¹², unsubstituted or substituted C₁-C₈-alkyl, C₂-C₆-alkenyl, C₁-C₈-haloalkyl; where m=1−4 where additionally or independently thereof two adjacent radicals R², R³ or R⁴, if appropriate via R¹² or R¹³, may form a 3- to 7-membered unsubstituted or substituted, saturated or unsaturated cycle which may contain no or up to four heteroatoms selected from the group consisting of N, O and S, where any two oxygen atoms are not adjacent to one another,
   where the substituents independently of one another are selected from the group consisting of hydrogen, fluorine, chlorine or bromine, C₁-C₄-alkyl, C₁-C₄-alkoxy, hydroxyl, oxo, C₁-C₄-haloalkyl and cyano,
R⁶ is hydrogen, C₁-C₂-alkyl, C₁-C₄-alkoxy(C₁-C₄)alkyl, C₁-C₄-trialkylsilyl, C₁-C₄-trialkylsilylethyl, C₁-C₄-dialkylmonophenylsilyl, formyl, (C₁-C₄alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulphinyl or C₁-C₂-alkylsulphonyl,
   where the substituents independently of one another are selected from the group consisting of hydrogen, fluorine, chlorine or bromine, C₁-C₄-alkyl, C₁-C₄-alkoxy, hydroxyl, C₁-C₄-haloalkyl and cyano,
R⁷ is hydrogen, C₁-C₃-alkyl, cyano or C₁-C₃-haloalkyl,
R⁸ is chlorine, bromine, iodine, cyano, methyl, CF₃, CCl₃, CFH₂ or CF₂H,
R⁹ is hydrogen, C₁-C₂-alkyl, C₁-C₄alkoxy(C₁-C₄)alkyl, C₁-C₆-trialkylsilyl, C₁-C₄-trialkylsilylethyl, C₁-C₄-dialkylmonophenylsilyl, (C₁-C₄-alkyl)carbonyl, (C₁-C₄-haloalkyl)carbonyl, (C₁-C₄-alkoxy)carbonyl, unsubstituted or substituted benzyl, unsubstituted or substituted C₂-C₆-alkenyl, unsubstituted or substituted C₂-C₆-alkynyl, C₁-C₆-alkylsulphinyl, C₁-C₆-alkylsulphonyl, C₁-C₆-haloalkylsulphinyl or C₁-C₆-haloalkylsulphonyl,
   where the substituents independently of one another are selected from the group consisting of fluorine, chlorine and/or bromine atoms, cyano, hydroxyl, methoxy, CF₃
R¹⁰ is hydrogen, methyl, trifluoromethyl, nitrile, phenyl, meta-chlorophenyl, meta-fluorophenyl, para-chlorophenyl, para-fluorophenyl, benzyl, meta-chlorobenzyl, COOH, COOMe or COOEt,
R^{11a} is hydrogen, phenyl, para-methoxyphenyl or COOMe,
R^{11b} is hydrogen, fluorine, phenyl, para-methoxyphenyl, COOH or COOEt,
R^{11c} is hydrogen or fluorine,
   where in each case only one of the radicals R¹⁰, R^{11a}, R^{11b} or R^{11c} is not hydrogen,
   or
R^{11b} and R^{11c} both represent fluorine.
R¹² are identical or different and are hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another
   or
   if two radicals R¹² are attached to a nitrogen atom, two radicals R¹² may form a 3- to 7-membered unsubstituted or substituted, saturated or unsaturated cycle which may contain up to four further heteroatoms selected from the group consisting of N, O and S, where any two oxygen atoms are not adjacent to one another,
   or
   if two radicals R¹² are adjacent in the grouping NR¹²COR¹² NR¹²SOR¹² NR¹²SO₂R¹² NR¹²SONR¹² NR¹²SO₂NR¹², two radicals R¹² may form a 3- to 7-membered unsubstituted or substituted, saturated or unsaturated cycle which may contain up to four further heteroatoms selected from the group consisting of N, O and S, where any two oxygen atoms are not adjacent to one another,
R¹³ are identical or different and are C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another,
   where two R¹³ may form a 3- to 7-membered unsubstituted or substituted, saturated or unsaturated cycle which may contain up to four further heteroatoms selected from the group consisting of N, O and S, where any two oxygen atoms are not adjacent to one another
   and where possible substituents are selected from the following list:
   fluorine chlorine, bromine, iodine, cyano, nitro, CF₃, CFH₂, CF₂H, C₂F₅, CCl₃, hydroxyl, OMe, OEt, OPr, OisoPr, OBu, OsecBu, OisoBu, OtertBu, O(CH₂)₂OCH₃, O(CH₂)₃OCH₃, O-cyclopentyl, O-phenyl, OCF₃, OCF₂H, OCF₂CF₃, OCF₂CF₂H, SH, SMe, SEt, SCF₃, SCF₂H, SPh, SCF₅, SO₂Me, SO₂CF₃, SOMe, SOEt, CO₂H, CO₂CH₃, CO₂Et, CO₂Pr, CO₂isoPr, CO₂tertBu, COMe, COCF₃, NH₂, NHMe, NMe₂, NHEt, NEt₂, NHPr, NHisoPr, NHnBu, NHtertBu, NHisoBu, NHsecBu, cyclopropylamino, formyl, CH₂CN, CHMeCN, CH₂COCH₃, CH₂OMe, (CH₂)₂OMe, (CH₂)₃OMe, CH₂OH, CH₂SMe, (CH₂)₂SMe, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, cyclohexylmethyl, neopentyl, prop-2-en-1-yl, 1-methylprop-2-en-1-yl, but-3-en-1-yl, (trimethylsilyl)methyl, cyclopropyl, cyclobutyl, cyclopentyl, cyclohexyl, phenyl, benzyl, —CH₂CH═CH₂, —CH(CH₃)CH═CH₂, —CH₂C≡CH,
   and also agrochemically active salts thereof.

The invention furthermore provides the use of the compounds of the formula (I) as fungicides.

The invention furthermore provides the use of the compounds of the formula (I) as insecticides.

The invention furthermore provides the use of the compounds of the formula (I) as herbicides.

Diaminopyrimidines of the formula (I) according to the invention and their agrochemically active salts are highly suitable as pesticides, in particular for controlling animal pests such as insects, parasites from the sub-class of the Acari (Acarina) (such as mites, spider mites and/or ticks) and/or nematodes. They are also suitable for controlling phytopathogenic harmful fungi. The compounds according to the invention mentioned above have in particular strong insecticidal and/or acaricidal and/or nematicidal and/or fungicidal activity and can be used both in crop protection, in the domestic and hygiene field and in the protection of materials.

The compounds of the formula (I) can be present both in pure form and as mixtures of various possible isomeric forms, in particular of stereoisomers, such as E and Z, threo and erythro, and also optical isomers, such as R and S isomers or atropisomers, and, if appropriate, also of tautomers. What is claimed are both the E and the Z isomers, and also the threo and erythro, and also the optical isomers, any mixtures of these isomers, and also the possible tautomeric forms.

Preference is given to using, as crop protection agents, compounds of the formula (I) in which one or more of the symbols have one of the following meanings:
X¹ is nitrogen or CR³
X² is nitrogen or CR⁴
   where X¹ and X² are not both nitrogen
R¹ and R⁵ independently of one another are H, C₁-C₂-alkyl, C₁-C₂-alkoxy, F, Cl or Br
R² to R⁴ independently of one another are hydrogen, fluorine, chlorine, bromine, iodine, cyano, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkynyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-haloalkyl), CONH(C₂-C₄-alkenyl), CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl, NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methyl cyclopropyl)carbonyl]-amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

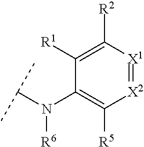

may be (2-oxo-2,3-dihydro-1H-indol-5-yl)amino, 1H-indol-6-ylamino, 1H-indol-5-ylamino, (2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-yl)amino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ is hydrogen, C₁-C₂-alkyl, triethylsilyl, trimethylsilyl, tert-butyldimethylsilyl, dimethyl-phenylsilyl, C₁-C₂-alkoxy(C₁-C₂)alkyl, formyl, (C₁-C₄-alkyl)carbonyl, (methoxymethyl)-carbonyl, (allyloxy)carbonyl, (cyclopropyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₄-alkenyl, unsubstituted or substituted C₂-C₄-alkynyl, C₁-C₂-alkylsulphinyl or C₁-C₂-alkylsulphonyl,
   where the substituents independently of one another are selected from the group consisting of hydrogen, fluorine, chlorine or bromine, C₁-C₄-alkyl, C₁-C₄-alkoxy, hydroxyl, C₁-C₄-haloalkyl or cyano,
R⁷ is hydrogen, methyl, CF₃, CFH₂, cyano, or CF₂H,
R⁸ is chlorine, bromine, iodine, cyano, methyl, CF₃, CCl₃, CFH₂, or CF₂H,
R⁹ is hydrogen, C₁-C₂-alkyl, C₁-C₂-alkoxy(C₁-C₂)alkyl, C₁-C₆-trialkylsilyl, C₁-C₄-trialkylsilylethyl, C₁-C₄-dialkylmonophenylsilyl, (C₁-C₄-alkyl)carbonyl, (C₁-C₄-haloalkyl)carbonyl, (C₁-C₄-alkoxy)carbonyl, benzyl, 4-methoxybenzyl, C₂-C₄-alkenyl, C₂-C₄-alkynyl, C₁-C₄-alkylsulphinyl, C₁-C₄-alkylsulphonyl, C₁-C₄-haloalkylsulphinyl or C₁-C₄-haloalkylsulphonyl,
R¹⁰ is hydrogen, methyl, trifluoromethyl, nitrile, phenyl, meta-chlorophenyl, meta-fluorophenyl, para-chlorophenyl, para-fluorophenyl, benzyl, meta-chlorobenzyl, COOH, COOMe or COOEt,
R^{11a} is hydrogen, phenyl, para-methoxyphenyl or COOMe,
R^{11b} is hydrogen, fluorine, phenyl, para-methoxyphenyl, COOH or COOEt,
R^{11c} is hydrogen or fluorine,
   where in each case only one of the radicals R¹⁰, R^{11a}, R^{11b} or R^{11c} is not hydrogen,
   or
R^{11b} and R^{11c} both represent fluorine.
R¹² are identical or different and are hydrogen, unsubstituted or substituted C₁-C₆-alkyl, unsubstituted or substituted C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₃-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another
   and also agrochemically active salts thereof.

Particular preference is given to using, as crop protection agents, compounds of the formula (I) in which one or more of the symbols have one of the following meanings:
X¹ is nitrogen or CR³
X² is nitrogen or CR⁴
   where X¹ and X² are not both nitrogen
R¹ and R⁵ independently of one another are H, C₁-C₂-alkyl, C₁-C₂-alkoxy, F, Cl or Br,
R² to R⁴ independently of one another are hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂ OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF₅, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH—CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C═CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(C₁-C₂-alkyl)CO(C₁-C₄-alkyl), NHCHO, NMeCHO, NHCO₂(C₁-C₄-alkyl), NHCO₂Ph, NHCO₂CH₂CH₂Cl, N(C₁-C₂-alkyl)CO₂(C₁-C₂-alkyl), NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂Cocyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3-dimethylbutyl, cyclohexylmethyl, C₂-C₆-alkenyl, (trimethylsilyl)methyl, C₁-C₂-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxo-pyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazo-lidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl-(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxo-tetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

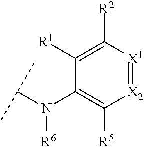

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ is hydrogen, C₁-C₂-alkyl, CH₂OCH₃, CH₂CH₂OCH₃, formyl, (C₁-C₄-alkyl)carbonyl, (C₁-C₄-haloalkyl)carbonyl, (C₁-C₄-alkoxy)carbonyl, COOC₂F₅, benzyloxycarbonyl, benzyl, 4-methoxybenzyl, 2,4-dimethoxybenzyl, 2-hydroxybenzyl, unsubstituted or substituted C₂-C₄-alkenyl, unsubstituted or substituted C₂-C₄-alkynyl, C₁-C₂-alkylsulphinyl or C₁-C₂-alkylsulphonyl,
   where the substituents independently of one another are selected from the group consisting of hydrogen, fluorine, chlorine or bromine, C₁-C₂-alkyl, C₁-C₂-haloalkyl or cyano,
R⁷ is hydrogen, methyl, CF₃, CFH₂, cyano,
R⁸ is chlorine, bromine, iodine, cyano, methyl, CF₃, CCl₃, CFH₂, or CF₂H,
R⁹ is hydrogen, C₁-C₂-alkyl, CH₂CH₂OCH₃, CH₂OCH₃, (C₁-C₂-alkyl)carbonyl, (C₁-C₂-haloalkyl)carbonyl, (C₁-C₄-alkoxy)carbonyl, benzyl, 4-methoxybenzyl, C₂-C₃-alkenyl, C₂-C₃-alkynyl, SOCH₃,═SO₂CH₃, SO₂CF₃ or SOCF₃,
R¹⁰ is hydrogen, methyl, trifluoromethyl, nitrile, phenyl, meta-chlorophenyl, meta-fluorophenyl, para-chlorophenyl, para-fluorophenyl, benzyl, meta-chlorobenzyl, COOH, COOMe or COOEt,
R^{11a} is hydrogen, phenyl, para-methoxyphenyl or COOMe,
R^{11b} is hydrogen, fluorine, phenyl, para-methoxyphenyl, COOH or COOEt,
R^{11c} is hydrogen or fluorine,
   where in each case only one of the radicals R¹⁰, R^{11a}, R^{11b} or R^{11c} is not hydrogen,
   or
R^{11b} and R^{11c} both represent fluorine,
   and also agrochemically active salts thereof.

Very particular preference is given to using, as crop protection agents, compounds of the formula (I) in which one or more of the symbols have one of the following meanings:
X¹ is nitrogen or CR³
X² is nitrogen or CR⁴
   where X¹ and X² are not both nitrogen
R¹ and R⁵ independently of one another are H, methyl, methoxy or F,
R² to R⁴ independently of one another are hydrogen, fluorine, chlorine, bromine, iodine, cyano, nitro, hydroxyl, O—C₁-C₄-alkyl, O(CH₂)₂OCH₃, O(CF₁₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH—CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH—CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C═CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(Cₑ-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, -ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

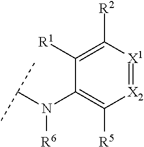

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, 2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ is hydrogen, C₁-C₂-alkyl, CH₂OCH₃, CH₂CH₂OCH₃, formyl, (C₁-C₂-alkyl)carbonyl, (C₁-C₂-haloalkyl)carbonyl, (C₁-C₄-alkoxy)carbonyl, COOC₂F₅, benzyloxycarbonyl, benzyl, 4-methoxybenzyl, CH₂CH═CH₂, CH₂C═CH, SOCH₃ or SO₂CH₃,
R⁷ is hydrogen, methyl, CF₃, CFH₂, cyano,
R⁸ is chlorine, bromine, iodine, cyano, methyl, CF₃, CCl₃, CFH₂, or CF₂H,
R⁹ is hydrogen, methyl, CH₂OCH₃, COMe, COCF₃, COOMe, COOEt, COOtertBu, benzyl, 4-methoxybenzyl, CH₂CH═CH₂ or CH₂CECH,
R¹⁰ is hydrogen, methyl or trifluoromethyl,
R^{11a} is hydrogen,
R^{11b} is hydrogen or fluorine,
R^{11c} is hydrogen or fluorine,
   where in each case only one of the radicals R¹⁰, R^{11a}, R^{11b} or R^{11c} is not hydrogen,

### Or

R^{11b} and R^{11c} both represent fluorine,
   and also agrochemically active salts thereof.

Special preference is given to using, as crop protection agents, compounds of the formula (I) in which one or more of the symbols have one of the following meanings:
X¹ is nitrogen or CR³
X² is nitrogen or CR⁴
   where X¹ and X² are not both nitrogen
R¹ and R⁵ independently of one another are H, methyl, methoxy or F,
R² to R⁴ independently of one another are hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH—CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), NHCOCF₃, N(C₂H₅)COMe, NHCOCH═CH₂, NHCOPh, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₅-cycloalkyl, 1-methoxycyclopropyl, 1-chlorocyclopropyl, 3-dimethylbutyl, cyclohexylmethyl, neopentyl, prop-2-en-1-yl, 1-methylprop-2-en-1-yl, but-3-en-1-yl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methyl-cyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl-(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydro furan-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl.
   and, if in each case two adjacent radicals R², R³ or R⁴, if appropriate via R¹² or R¹³, form a cycle, the following subunit from the general formula (I):

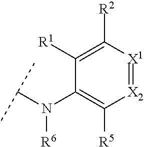

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ is hydrogen, Me, formyl, COMe, COCF₃, COOMe, COOEt, COOtertBu, benzyloxycarbonyl or benzyl,
R⁷ is hydrogen, methyl, CF₃,
R⁸ is chlorine, bromine, iodine, cyano, methyl, CF₃, CCl₃, CFH₂,
R⁹ is hydrogen, methyl,
R¹⁰ is hydrogen or methyl,
R^{11a} is hydrogen,
R^{11b} is hydrogen or fluorine,
R^{11c} is hydrogen or fluorine,
   where in each case only one of the radicals R¹⁰, R^{11a}, R^{11b} or R^{11c} is not hydrogen,
   or
R^{11b} and R^{11c} both represent fluorine,
   and also agrochemically active salts thereof.

Special preference is furthermore given to using, as crop protection agents, compounds of the formula (I) in which one or more of the symbols have one of the following meanings:
X¹ is nitrogen or CR³
X² is nitrogen or CR⁴
   where X¹ and X² are not both nitrogen,
R¹ and R⁵ independently of one another are H, methyl, methoxy or F,
R² to R⁴ independently of one another are hydrogen, fluorine, chlorine, bromine, iodine, cyano, nitro, hydroxyl, OMe, OEt, OPr, OisoPr, OBu, OsecBu, OisoBu, OtertBu, O(CH₂)₂OCH₃, O(CH₂)₃OCH₃, O-cyclopentyl, OCF₃, OCF₂CF₃, OCF₂CF₂H, O(CH₂)₂N(CH₃)₂, OCH(CH₃)CH₂OCH₃, OSO₂NMe₂, OCONHMe, OCONHEt, OCONHPr, OCONHisoPr, OCONMe₂, OCONEt₂, OCONPr₂, OCONisoPr₂, OCOMe, OCOEt, OCOBu, OSO₂N(CH₃)₂, SH, SF₅, SMe, SEt, SiPr, SCF₃, SCF₂H, SPh, SO₂Me, SO₂CF₃, SO₂NH₂, SO₂NHMe, SO₂NMe₂, SO₂NHPh, SO₂NHnBu, SO₂NHtBu, COMe, COEt, COPr, COCF₃, COCH₂CN, CONHMe, CONMe₂, CONHEt, CONHCH₂CF₃, CONEt₂, CONHPr, CONHisoPr, CONHBu, CONHtertBu, CONHCH₂CH═CH₂, CONHCH₂C═CH, CONHCH₂C(═CH₂)CH₃, CONHCH(CH₃)CH₂OCH₃, CONH(CH₂)₂OCH₃, CONHPh, CONH-cyclopropyl, CONH-cyclopropylmethyl, piperidin-1-ylcarbonyl, morpholin-4-ylcarbonyl, COOH, COCl, CO₂CH₃, CO₂Et, CO₂Pr, CO₂isoPr, CO₂(CH₂)₂OCH₃, NHCOMe, NHCOEt, NHCOPr, NHCOisoPr, NHCOBu, NHCOisoBu, NHCOCF₃, NHCOsecBu, NHCOisoBu, NHCOtertBu, N(C₂H₅)COMe, NHCOCH═CH₂, NHCOPh, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, NHCHO, NMeCHO, NHCO₂Me, NHCO₂Et, NHCO₂ isoPr, NHCO₂tertBu, NHCO₂ isoBu, NHCO₂secBu, NHCO₂nBu, NHCO₂Ph, NHCO₂CH₂CH₂Cl, NMeCO₂Me, NH₂, NHMe, NMe₂, NHEt, NEt₂, NHPr, NHisoPr, NHnBu, cyclopropylamino, NHCH(CH₃)CH₂OCH₃, acetyl(cyclopropyl)amino, [(1-methyl cyclopropyl)carbonyl]amino, morpholin-1-yl, morpholin-4-ylmethyl, NHSO₂Me, CH₂CN, CHMeCN, CH₂SO₂Me, CH₂SO₂NHMe, CH₂SO₂NHPr, CH₂COCH₃, CH₂COtertBu, CH(CH₃)COCH₃, CH₂COCH(CH₃)₂, CH₂CO-cyclopropyl, CH₂CONHtertBu, CH₂CO₂Et, CH₂OMe, (CH₂)₂OMe, C(CH₃)₂OCH₃, CH₂OisoPr, CH₂OtertBu, CHCF₃OH, CH₂OH, CH₂SMe, CH₂NHCOOMe, CH₂NHAc, CH₂NHCOCF₃, CH₂NMe₂, CH₂NH(CH₂)₂OCH₃, (CH₂)₂NMe₂, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, cyclohexylmethyl, neopentyl, prop-2-en-1-yl, 1-methylprop-2-en-1-yl, but-3-en-1-yl, (trimethylsilyl)methyl, CF₃, CF₂H, morpholin-4-ylsulphonyl, 2-oxopyrrolidin-1-yl, 2-oxo-1,3-oxazolidin-3-yl, (3-methyl-2,5-dioxoimidazolidin-1-yl), 2,5-dioxopyrrolidin-1-yl, 3-methyl-2-oxoimidazolidin-1-yl, 2,5-dioxoimidazolidin-4-yl, 2-thienyl, piperidin-1-ylsulphonyl, 1,3-thiazol-2-yl, 1,3-thiazol-4-yl, [(4-methylphenyl)amino]sulphonyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl-) amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, -methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

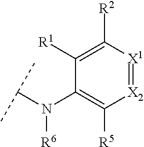

may be (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (2-oxo-2,3,4,5-tetrahydro-1H-1-benzazepin-8-yl)amino, 1,3-benzodioxol-5-ylamino, (1,3-dioxo-2,3-dihydro-1H-isoindol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino,
R⁶ is hydrogen, Me, formyl, COMe, COOMe, COOEt, COOtertBu, COOBn, COCF₃, benzyl,
R⁷ is hydrogen, methyl,
R⁸ is chlorine, bromine, iodine, cyano, CF₃, CFH₂,
R⁹ is hydrogen, methyl,
R¹⁰ is hydrogen or methyl,
R^{11a} is hydrogen,
R^{11b} is hydrogen,
R^{11c} is hydrogen,
   where in each case only one of the radicals R¹⁰, R^{11a}, R^{11b} or R^{11c} is not hydrogen,
   or
R^{11b} and R^{11c} both represent fluorine,
   and also agrochemically active salts thereof.

Preference is furthermore given to using, as crop protection agents, compounds of the formula (I) in which one or more of the symbols have one of the following meanings:
X¹ is CR³ and
X² is CR⁴,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
X¹ is CR³ and
X² is nitrogen,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R¹⁰ is H or Me,
R^{11a,b,c} is in each case H,
is CR³ and
X² is CR⁴,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R⁶ is H, CHO, COCH₃ or COCF₃,
R² is H
R⁹ is H, Me, CHO or COCH₃,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R⁶ is H,
R⁷ is H
R⁹ is H or Me,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R¹ is H,
R⁵ is H
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R¹ is H,
R⁵ is H
X¹ is CR³ and
X² is CR⁴,
   where the other substituents have one or more of the meanings mentioned above,
   and also the agrochemically active salts thereof.

Preference is furthermore given to using, as crop protection agents, compounds of the formula (I) in which one or more of the symbols have one of the following meanings:
R⁸ is chlorine, bromine, CF₃,
   where the other substituents have one or more of the meanings mentioned above,
   and also the agrochemically active salts thereof.

The invention also provides compounds formulae (Ia), (Ib) and (Ic).

Compounds of the formulae (Ia), (Ib) and (Ic) according to the invention and their agrochemically active salts are highly suitable as pesticides, in particular for controlling animal pests such as insects, parasites from the sub-class of the Acari (Acarina) (such as mites, spider mites and/or ticks) and/or nematodes. They are also suitable for controlling phytopathogenic harmful fungi. The compounds according to the invention mentioned above have in particular strong insecticidal and/or acaricidal and/or nematicidal and/or fungicidal activity and can be used both in crop protection, in the domestic and hygiene field and in the protection of materials. Furthermore, surprisingly, they have herbicidal activity.

The invention also provides compounds of the formula (Ia).

a) compounds of the formula (Ia),

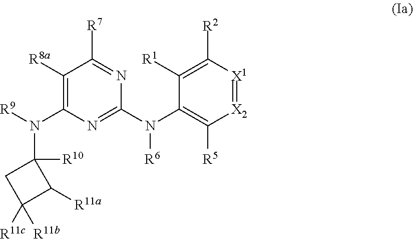

in which
R^{8a} represents chlorine, iodine, CFH₂, CF₂H, CCl₃, cyano or Me and
X¹, X², R¹ to R⁷, R^{1-A}, R⁹ to R¹³ have the general, preferred, particularly preferred, very particularly preferred and especially preferred meanings indicated above, and also agrochemically active salts of these compounds.

Preference is furthermore given to compounds of the formula (Ia) in which one or more of the symbols have one of the following meanings:
X¹ is CR³ and
X² is CR⁴,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR² and
X² is nitrogen,
   where the other substituents have one or more of the meanings mentioned above,
   and also the agrochemically active salts thereof.

Preference is furthermore given to compounds of the formula (Ia) in which one or more of the symbols have one of the following meanings:
R¹⁰ is H or Me,
R^{11a} is H,
R^{11,b,c} is in each case H or Me,
   where the other substituents have one or more of the meanings mentioned above,
   and also the agrochemically active salts thereof.

Preference is furthermore given to compounds of the formula (Ia) in which one or more of the symbols have one of the following meanings:
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
R¹⁰ is H or Me,
R^{11a,b,c} is in each case H,
X¹ is CR³ and
X² is CR⁴,
   where the other substituents have one or more of the meanings mentioned above,
   and also the agrochemically active salts thereof.

Preference is furthermore given to compounds of the formula (Ia) in which one or more of the symbols have one of the following meanings:
R⁶ is H, CHO, COCH₃ or COCF₃,
R⁷ is H
R⁹ is H, Me, CHO or COCH₃,
   where the other substituents have one or more of the meanings mentioned above,
   and also the agrochemically active salts thereof.

Preference is furthermore given to compounds of the formula (Ia) in which one or more of the symbols have one of the following meanings:
R⁶ is H,
R⁷ is H
R⁹ is H or Me,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
X² is CR⁴,
R⁶ is H, CHO, COCH₃ or COCF₃,
R⁷ is H
R⁹ is H, Me, CHO or COCH₃,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
X² is CR⁴,
R⁶ is H,
R⁷ is H
R⁹ is H or Me,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
X² is CR⁴,
R⁶ is H, CHO, COCH₃ or COCF₃,
R⁷ is H
R⁹ is H, Me, CHO or COCH₃,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
X² is CR⁴,
R⁶ is H,
R⁷ is H
R⁹ is H or Me,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
X¹ is CR³ and
X² is CR⁴,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
R⁶ is H, CHO, COCH₃ or COCF₃,
R⁷ is H,
R⁹ is H, Me, CHO or COCH₃,
X¹ is CR³ and
X² is CR⁴,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
R⁶ is H, CHO, COCH₃ or COCF₃,
R⁷ is H,
R⁹ is H, Me, CHO or COCH₃,
R¹⁰ is H or Me,
R^{11a,b,c} is in each case H,
X¹ is CR³ and
X² is CR⁴,
   where the other substituents have one or more of the meanings mentioned above,
   and also the agrochemically active salts thereof.

Preference is furthermore given to compounds of the formula (Ia) in which one or more of the symbols have one of the following meanings:
X² is CR⁴,
R⁴ is hydrogen, fluorine, chlorine, bromine, iodine, cyano, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-halo alkyl), CONH(C₂-C₄-alkenyl), CONH(C₂-C₄-alkynyl), CONHCH₂C(—CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONN-cyclopropylmethyl, piperidin-1-ylcarbonyl, morpholin-4-ylcarbonyl, (4-methylpiperazin-1-yl)carbonyl, COOH, COC₁, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)o(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methylcyclopropyl)carbonyl]-amino, morpholin-1-yl, morpholin-4-ylmethyl, NHSO(C₁-C₄-alkyl), NHSo(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

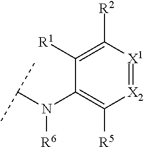

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ia) in which one or more of the symbols have one of the following meanings:
X² is CR⁴,
R⁴ is H or SO₂N(R¹²)₂,
R¹² are identical or different and are hydrogen, unsubstituted or substituted C₁-C₆-alkyl, unsubstituted or substituted C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₃-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another,
   where the other substituents have one or more of the meanings mentioned above,
   and also the agrochemically active salts thereof.

Preference is furthermore given to compounds of the formula (Ia) in which one or more of the symbols have one of the following meanings:
X² is CR⁴,
R⁴ is H, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂ or SO₂NHCH₂CH═CH₂,
   where the other substituents have one or more of the meanings mentioned above,
   and also the agrochemically active salts thereof.

Preference is furthermore given to compounds of the formula (Ia) in which one or more of the symbols have one of the following meanings:
X² is CR⁴,
R⁴ is H or SO₂NH₂,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R⁴ is H or SO₂N(R¹²)₂,
R¹² are identical or different and are hydrogen, unsubstituted or substituted C₁-C₆-alkyl, unsubstituted or substituted C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₃-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R⁴ is H, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂ or SO₂NHCH₂CH═CH₂,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R⁴ is H or SO₂NH₂,
   where the other substituents have one or more of the meanings mentioned above,
   and also the agrochemically active salts thereof.

Preference is furthermore given to compounds of the formula (Ia) in which one or more of the symbols have one of the following meanings:
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
R¹⁰ is H or Me,
R^{11a,b,c} is in each case H,
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
R⁶ is H, CHO, COCH₃ or COCF₃,
R⁷ is H
R⁹ is H, Me, CHO or COCH₃,
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
R¹ is H,
R⁵ is H,
R^{8a} is chlorine, iodine or cyano,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R¹ is H,
R⁵ is H,
R^{8a} is chlorine, iodine or cyano,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R¹ is H,
R⁵ is H,
R^{8a} is chlorine, iodine or cyano,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
R^{8a} is chlorine,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R¹ is H,
R⁵ is H,
R^{8a} is chlorine,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³ and
X² is CR⁴,
R¹ is H,
R⁵ is H,
R^{8a} is chlorine,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
R³ is hydrogen, fluorine, chlorine, bromine, iodine, cyano, nitro, hydroxyl, O—C₁-C₄-alkyl, O—(C₁-C₃-haloalkyl), —(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₃-haloalkyl, SPh, SO(C₁-C₄-SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-haloalkyl), CONH(C₂-C₄-alkenyl), CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methyl cyclopropyl)carbonyl]-amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(CCH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

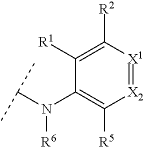

may be (2-oxo-2,3-dihydro-1H-indol-5-yl)amino, 1H-indol-6-ylamino, 1H-indol-5-yl amino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-yl amino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
R³ is H, F, I, methoxy, methyl, CN, cyanomethyl, cyanoethyl, acetyl, acetamido, trifluoroacetylamino, acetyl(methyl)amino, acetyl(ethyl)amino, methoxycarbonylamino, ethoxycarbonylamino, methylthio, methylsulphonyl, methylaminosulphonyl, dimethylaminosulphonyl, butylaminosulphonyl, aminosulphonyl, 4-tert-butoxycarbonylpiperazin-1-yl, 2,2-dimethylpropanoylamino, [(1-methylcyclopropyl)carbonyl]amino, methylcarbamoyl, 2-oxo-1,3-oxazolidin-3-yl, 2-oxopyrrolidin-1-yl, 2,5-dioxoimidazolidin-4-yl, piperidin-1-ylcarbonyl, morpholin-4-ylcarbonyl, dimethylcarbamoyl, ethylmethylcarbamoyl, diethylcarbamoyl, propan-2-ylcarbamoyl, prop-2-en-1-ylcarbamoyl, 2-oxopropyl,
   where the other substituents have one or more of the meanings mentioned above,
   and also the agrochemically active salts thereof.

Preference is furthermore given to compounds of the formula (Ia) in which one or more of the symbols have one of the following meanings:
R² is hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH—CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C═CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONN-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

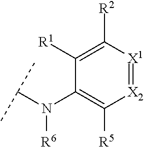

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ia) in which one or more of the symbols have one of the following meanings:
R² is H, methyl, ethyl, propan-2-yl, tert-butyl, cyanomethyl, aminosulphonyl, methylsulphonyl, acetamido, 3-oxobutyl, morpholin-4-ylcarbonyl, C₁, CF₃, CN, methoxy, methoxymethyl, trifluoromethoxy, 2,2,2-trifluoro-1-hydroxyethyl, methylthio, ethylthio, isopropylthio, methylamino, acetyl(methyl)amino, 2-oxopyrrolidin-3,5-dimethylpiperidin-1-yl, 2-oxopiperidin-1-yl, 2-oxopyrrodin-1-yl, 2-oxazepan-1-yl, 3-methyl-2-oxoimidazolidin-1-yl, 1,1-dioxidoisothiazolidin-2-yl, methacryloylamino, 2,2-dimethylpropanoylamino, methylcarbamoyl, dimethylcarbamoyl, piperidin-1-ylsulphonyl, ethoxycarbonylamino, methylcarbamoyloxy, methoxycarbonylamino, [(1-methylcyclopropyl)carbonyl]amino, (3-fluoro-2,2-dimethylpropanoyl)amino,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
R² and R³ independently of one another are hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF₅, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonyl amino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

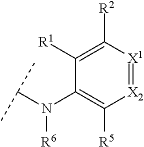

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-ylamino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ia) in which one or more of the symbols have one of the following meanings:
X¹ is CR³,
R² is H, methyl, ethyl, propan-2-yl, tert-butyl, cyanomethyl, aminosulphonyl, methylsulphonyl, acetamido, 3-oxobutyl, morpholin-4-ylcarbonyl, C₁, CF₃, CN, methoxy, methoxymethyl, trifluoromethoxy, 2,2,2-trifluoro-1-hydroxyethyl, methylthio, ethylthio, isopropylthio, methylamino, acetyl(methyl)amino, 2-oxopyrrolidin-3,5-dimethylpiperidin-1-yl, 2-oxopiperidin-1-yl, 2-oxopyrrodin-1-yl, 2-oxazepan-1-yl, 3-methyl-2-oxoimidazolidin-1-yl, 1,1-dioxidoisothiazolidin-2-yl, methacryloylamino, 2,2-dimethylpropanoylamino, methylcarbamoyl, dimethylcarbamoyl, piperidin-1-ylsulphonyl, ethoxycarbonylamino, methylcarbamoyloxy, methoxycarbonylamino, [(1-methylcyclopropyl)carbonyl]amino, (3-fluoro-2,2-dimethylpropanoyl)amino,
R³ is H, F, I, methoxy, methyl, CN, cyanomethyl, cyanoethyl, acetyl, acetamido, trifluoroacetylamino, acetyl(methyl)amino, acetyl(ethyl)amino, methoxycarbonylamino, ethoxycarbonylamino, methylthio, methylsulphonyl, methylaminosulphonyl, dimethylaminosulphonyl, butylaminosulphonyl, aminosulphonyl, 4-tert-butoxycarbonylpiperazin-1-yl, 2,2-dimethylpropanoylamino, [(1-methylcyclopropyl)carbonyl]amino, methylcarbamoyl, 2-oxo-1,3-oxazolidin-3-yl, 2-oxopyrrolidin-1-yl, 2,5-dioxoimidazolidin-4-yl, piperidin-1-ylcarbonyl, morpholin-4-ylcarbonyl, dimethylcarbamoyl, ethylmethylcarbamoyl, diethylcarbamoyl, propan-2-ylcarbamoyl, prop-2-en-1-ylcarbamoyl, 2-oxopropyl,
   where the other substituents have one or more of the meanings mentioned above,
   and also the agrochemically active salts thereof.

The radical definitions mentioned above can be combined with one another as desired. Moreover, individual definitions may not apply.

The invention also provides compounds of the formulae (Ib).

b) compounds of the formula (Ib),

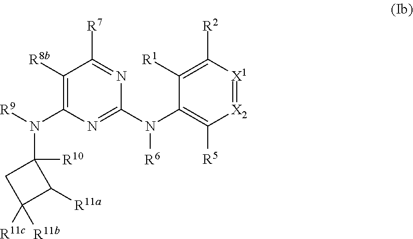

in which the symbols are as defined below:
R^{8b} represents CF₃ and
X¹, X², R¹ to R⁷, R^{1-A}, R⁹, R¹⁰, R^{11a,b,c,}, R¹² and R¹³ have the general, preferred, particularly preferred, very particularly preferred and especially preferred meanings indicated above,
   except for those cases where in the following subunit of the general formula below (Ib)

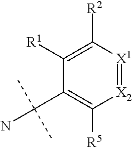

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   and also agrochemically active salts of these compounds.

Preference is furthermore given to compounds of the formula (Ib) in which one or more of the symbols have one of the following meanings:
X¹ is CR³ and
X² is CR⁴,
   except for those cases where in the following subunit of the general formula (Ib) below

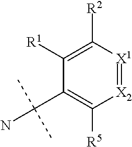

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³ and
   except for the following case:
R³ is CON(R¹²)₂ and both radicals R¹² together with the nitrogen atom to which they are attached form a 4-methyl-1,4-piperazinyl radical;
X² is nitrogen,
   where the other substituents have one or more of the meanings mentioned above,
   and also the agrochemically active salts thereof.

Preference is furthermore given to compounds of the formula (Ib) in which one or more of the symbols have one of the following meanings:
R¹⁰ is H or Me,
R^{11a} is H,
R^{11,b,c} is in each case H or Me,
   where the other substituents have one or more of the meanings mentioned above,
   and also the agrochemically active salts thereof.

Preference is furthermore given to compounds of the formula (Ib) in which one or more of the symbols have one of the following meanings:
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ib) in which one or more of the symbols have one of the following meanings:
R¹⁰ is H or Me,
R^{11a,b,c} is in each case H,
X¹ is CR³ and
X² is CR⁴,
   except for those cases where in the following subunit of the general formula (Ib) below

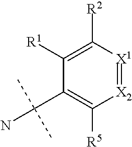

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (M-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R⁶ is H, CHO, COCH₃ or COCF₃,
R⁷ is H
R⁹ is H, Me, CHO or COCH₃,
   except for those cases where in the following subunit of the general formula below (Ib)

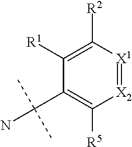

X¹ represents CR³ and X² represents CR⁴ and
   either R² and R³ or R³ and R⁴ together with the phenyl ring a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R⁶ is H,
R⁷ is H
R⁹ is H,
   except for those cases where in the following subunit of the general formula (Ib) below

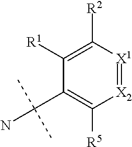

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
X² is CR⁴,
R⁶ is H,
R⁷ is H
R⁹ is H,
   except for those cases where in the following subunit of the general formula (Ib) below

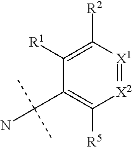

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
X² is CR⁴,
R⁶ is H,
R⁷ is H
R⁹ is H,
R¹⁰ is H or Me,
R^{11a,b,c} is in each case H,
   except for those cases where in the following subunit of the general formula (Ib) below

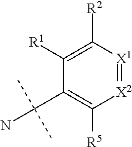

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R¹ is H,
R⁵ is H
   except for those cases where in the following subunit of the general formula (Ib) below

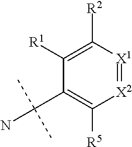

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (11-1-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R¹ is H,
R⁵ is H
R¹⁰ is H or Me,
R^{11a,b,c} is in each case H,
   except for those cases where in the following subunit of the general formula (Ib) below

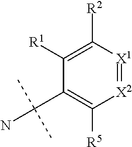

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R¹ is H,
R⁵ is H,
X¹ is CR³ and
X² is CR⁴,
   except for those cases where in the following subunit of the general formula (Ib) below

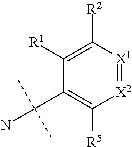

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R¹ is H,
R⁵ is H,
R⁶ is H,
R⁷ is H,
R⁹ is H,
X¹ is CR³ and
X² is CR⁴,
   except for those cases where in the following subunit of the general formula (Ib) below

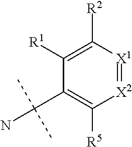

X¹ represents CR³ and X² represents CR⁴ and
   either R² and R³ or R³ and R⁴ together with the phenyl ring form a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
R¹ is H,
R⁵ is H,
R⁶ is H,
R⁷ is H,
R⁹ is H,
R¹⁰ is H or Me,
R^{11a,b,c} is in each case H,
X¹ is CR³ and
X² is CR⁴,
   except for those cases where in the following subunit of the general formula (Ib) below

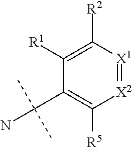

X¹ represents CR³ and X² represents CR⁴ and
   either R² and R³ or R³ and R⁴ together with the phenyl ring forth a (1H-2-hydroxyindol-6-yl), (1H-2-hydroxyindol-7-yl) or (1H-3,4-dihydro-2-oxoquinolin-7-yl) radical;
   or R³ is CON(R¹²)₂ and both radicals R¹² together with the nitrogen atom to which they are attached form a 4-methyl-1,4-piperazinyl radical;
   where the other substituents have one or more of the meanings mentioned above,
   and also the agrochemically active salts thereof.

Preference is furthermore given to compounds of the formula (Ib) in which one or more of the symbols have one of the following meanings:
X² is CR⁴,
R⁴ is hydrogen, fluorine, chlorine, bromine, iodine, cyano, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-haloalkyl), CONH(C₂-C₄-alkenyl), CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘ(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methylcyclopropyl)carbonyl]-amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

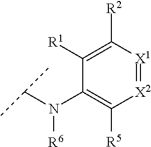

may be (2-oxo-2,3-dihydro-1H-indol-5-yl)amino, 1H-indol-6-ylamino, 1H-indol-5-yl amino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ib) in which one or more of the symbols have one of the following meanings:
X² is CR⁴,
R⁴ is H,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³ and
R³ is hydrogen, fluorine, chlorine, bromine, iodine, cyano, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-halo alkyl), CONH(C₂-C₄-alkenyl), CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONN-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C—S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methylcyclopropyl)carbonyl]-amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cyclo alkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-halo alkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

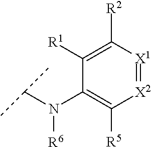

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
X² is CR⁴,
R⁴ is H,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
R³ is hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C═CHSO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂ (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methyl cyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

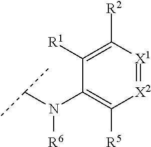

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benz-imidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
R³ is H, F, dimethylcarbamoyl, prop-2-en-1-ylcarbamoyl, 2-oxopyrrolidin-1-yl or piperidin-1-ylcarbonyl,
   where the other substituents have one or more of the meanings mentioned above,
   and also the agrochemically active salts thereof.

Preference is furthermore given to compounds of the formula (Ib) in which one or more of the symbols have one of the following meanings:
R² is hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, CH(CH₃)CH₂OCH₃, SO₂NMe₂, OCONH(C₁-C₃-alkyl), OCON(C₁-C₃-alkyl)₂, OCO(C₁-C₄-alkyl), OSO₂N(CH₃)₂, SH, SF₅, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C═CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₃-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2, -dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

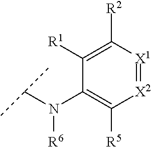

may be (2-oxo-2,3-dihydro-1H-indol-5-yl)amino, 1H-indol-6-ylamino, 1H-indol-5-yl amino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-yl amino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ib) in which one or more of the symbols have one of the following meanings:
R² is H, 2-oxopyrrolidin-1-yl, acetyl, cyanomethyl, ethylsulphanyl, methylthio, methylsulphonyl, methoxy, propan-2-yl or tert-butyl,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
R² and R³ independently of one another are hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, 3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

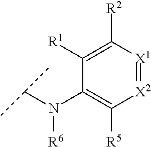

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
R² is H, 2-oxopyrrolidin-1-yl, acetyl, cyanomethyl, ethylsulphanyl, methylthio, methylsulphonyl, methoxy, propan-2-yl or tert-butyl,
R³ is H, F, dimethylcarbamoyl, prop-2-en-1-ylcarbamoyl, 2-oxopyrrolidin-1-yl or piperidin-1-ylcarbonyl,
   where the other substituents have one or more of the meanings mentioned above,
   and also the agrochemically active salts thereof.

The radical definitions mentioned above can be combined with one another as desired. Moreover, individual definitions may not apply.

The invention also provides compounds of the formulae (Ic).

c) compounds of the formula (Ic),

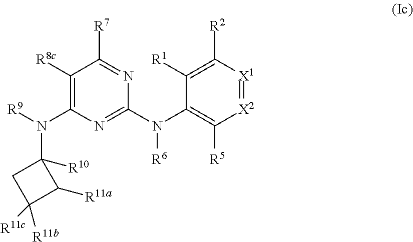

in which the symbols have the following meanings:
R^{8c} represents Br
   and
X¹, X², R⁶, R⁷ R^{1-A}, R⁹, R¹⁰, R^{11a,b,c,}, R¹² and R¹³ have the general, preferred, particularly preferred, very particularly preferred and especially preferred meanings indicated above, and also agrochemically active salts of these compounds,
   except for the following cases:
   either X¹ represents CR³ and R² and R³ form, in the following subunit from the general formula (IC):

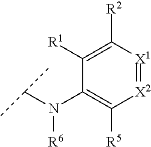

a (1H-indazol-6-yl)amine,
or X¹ represents CR³ and X² represents CR⁴ and R⁴ and R³, in the above subunit from the general formula (Ic), also form a (1H-indazol-6-yl)amine.

Preference is furthermore given to compounds of the formula (Ic) in which one or more of the symbols have one of the following meanings:
X¹ is CR³ and
X² is CR⁴,
   except for the following cases:
   either X¹ represents CR³ and R² and R³ form, in the following subunit from the general formula (Ic):

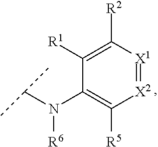

a (1H-indazol-6-yl)amine,
or X¹ represents CR³ and X² represents CR⁴ and R⁴ and R³, in the above subunit from the general formula (Ic), also form a (1H-indazol-6-yl)amine,
where the other substituents have one or more of the meanings mentioned above,
and also the agrochemically active salts thereof.

Preference is furthermore given to compounds of the formula (Ic) in which one or more of the symbols have one of the following meanings:
X¹ is CR³ and
X² is nitrogen,
   except for the following case:
X¹ represents CR³ and R² and R³ form, in the following subunit from the general formula (Ic):

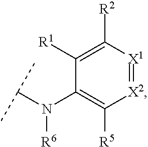

a (1H-indazol-6-yl)amine,
where the other substituents have one or more of the meanings mentioned above,
and also the agrochemically active salts thereof.

Preference is furthermore given to compounds of the formula (Ic) in which one or more of the symbols have one of the following meanings:
R¹⁰ is H or Me,
R^{11a} is H,
R^{11,b,c} is in each case H or Me,
   where the other substituents have one or more of the meanings mentioned above,
   and also the agrochemically active salts thereof.

Preference is furthermore given to compounds of the formula (Ic) in which one or more of the symbols have one of the following meanings:
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ic) in which one or more of the symbols have one of the following meanings:
R¹⁰ is H or Me,
R^{11a,b,c} is in each case H,
X¹ is CR³ and
X² is CR⁴,
   except for the following cases:
   either X¹ represents CR³ and R² and R³ form, in the following subunit from the general formula (Ic):

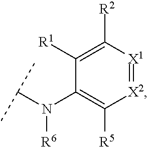

a (1H-indazol-6-yl)amine,
or X¹ represents CR³ and X² represents CR⁴ and R⁴ and R³, in the above subunit from the general formula (Ic), also form a (1H-indazol-6-yl)amine, where the other substituents have one or more of the meanings mentioned above, and also the agrochemically active salts thereof.

Preference is furthermore given to compounds of the formula (Ic) in which one or more of the symbols have one of the following meanings:
R⁶ is H, CHO, COCH₃ or COCF₃,
R⁷ is H,
R⁹ is H, Me, CHO or COCH₃,
   where the other substituents have one or more of the meanings mentioned above,
   and also the agrochemically active salts thereof.

Preference is furthermore given to compounds of the formula (Ic) in which one or more of the symbols have one of the following meanings:
R⁶ is H,
R⁷ is H,
R⁹ is H,
   where the other substituents have one or more of the meanings mentioned above,
   and also the agrochemically active salts thereof.

Preference is furthermore given to compounds of the formula (Ib) in which one or more of the symbols have one of the following meanings:
X¹ is CR³,
X² is CR⁴,
R⁶ is H,
R⁷ is H
R⁹ is H,
   where the other substituents have one or more of the meanings mentioned above,
   and also the agrochemically active salts thereof.

Preference is furthermore given to compounds of the formula (Ic) in which one or more of the symbols have one of the following meanings:
X¹ is CR³,
X² is CR⁴,
R⁶ is H,
R⁷ is H
R⁹ is H,
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ic) in which one or more of the symbols have one of the following meanings:
R¹ is H,
R⁵ is H
   where the other substituents have one or more of the meanings mentioned above,
   and also the agrochemically active salts thereof.

Preference is furthermore given to compounds of the formula (Ic) in which one or more of the symbols have one of the following meanings:
R¹ is H,
R⁵ is H
R¹⁰ is H or Me,
R^{11a,b,c} is in each case H,
   where the other substituents have one or more of the meanings mentioned above,
   and also the agrochemically active salts thereof.

Preference is furthermore given to compounds of the formula (Ic) in which one or more of the symbols have one of the following meanings:
R¹ is H,
R⁵ is H
X¹ is CR³ and
X² is CR⁴,
   where the other substituents have one or more of the meanings mentioned above,
   and also the agrochemically active salts thereof.

Preference is furthermore given to compounds of the formula (Ic) in which one or more of the symbols have one of the following meanings:
R¹ is H,
R⁵ is H,
R⁶ is H,
R⁷ is H,
R⁹ is H,
X¹ is CR³ and
X² is CR⁴,
   where the other substituents have one or more of the meanings mentioned above,
   and also the agrochemically active salts thereof.

Preference is furthermore given to compounds of the formula (Ic) in which one or more of the symbols have one of the following meanings:
R¹ is H,
R⁵ is H,
R⁶ is H,
R⁷ is H,
R⁹ is H,
R¹⁰ is H or Me,
R^{11a,b,c} is in each case H,
X¹ is CR³ and
X² is CR⁴,
   where the other substituents have one or more of the meanings mentioned above,
   and also the agrochemically active salts thereof.

Preference is furthermore given to compounds of the formula (Ic) in which one or more of the symbols have one of the following meanings:
X² is CR⁴,
R⁴ is hydrogen, fluorine, chlorine, bromine, iodine, cyano, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-haloalkyl), CONH(C₂-C₄-alkenyl), CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methylcyclopropyl)carbonyl]-amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methyl-ethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazoli-din-2-yl, 3-6-methyl-1,1-dioxido-1,2,6-thiadiazinan-2-yl,
   where m=1−3
   and, if in each case two adjacent radicals R², R³ or R⁴, if appropriate via R¹² or R¹³, form a cycle, the following subunit from the general formula (I):

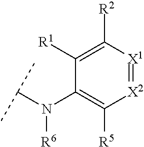

may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ic) in which one or more of the symbols have one of the following meanings:
X² is CR⁴,
R⁴ is H, halogen, C₁-C₄-alkyl or O—C₁-C₄-alkyl,
   where the other substituents have one or more of the meanings mentioned above,
   and also the agrochemically active salts thereof.

Preference is furthermore given to compounds of the formula (Ic) in which one or more of the symbols have one of the following meanings:
X² is CR⁴,
R⁴ is H, halogen, methyl or methoxy,
   where the other substituents have one or more of the meanings mentioned above,
   and also the agrochemically active salts thereof.

Preference is furthermore given to compounds of the formula (Ic) in which one or more of the symbols have one of the following meanings:
X¹ is CR³,
R³ is hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF_{S}, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONN-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonyl amino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, (2,2,2-trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

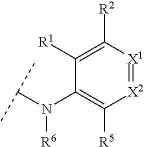

may be (2-oxo-2,3-dihydro-1H-indol-5-yl)amino, 1H-indol-6-ylamino, 1H-indol-5-yl amino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ic) in which one or more of the symbols have one of the following meanings:
X¹ is CR³,
R³ is H, halogen, CN, COOH, methoxycarbonyl, pentanoyloxy, OH, methoxy, ethoxy, isopropoxy, methylthio, difluoromethylthio, methylsulphonyl, trifluoromethylsulphonyl, methylamino, methyl, isopropyl, 2-methoxy-1-methylethoxy, acetyl(cyclopropyl)amino, acetyl(cyclohexyl)amino, (1-methylcyclopropyl)carbonylamino, methoxycarbonylamino, (ethoxycarbonyl)amino, (tert-butoxycarbonyl)amino, [(2-chloroethoxy)carbonyl]-amino, (3-chloro-2,2-dimethylpropanoyl)amino, (3-fluoro-2,2-dimethylpropanoyl)amino, isobutyrylamino, 3-methyl,-2,5-dioxoimidazolidin-1-yl, 2,5-dioxoimidazolidin-4-yl, 3-oxomorpholin-4-yl, 2-oxo-1,3-oxazolidin-3-yl, 2-oxopyrrolidin-1-yl, 2,5-dioxopyrrolidin-1-yl, 1,3-thiazol-4-yl, CF₃, difluoromethoxy, trifluoromethoxy, 1,1,2,2-tetrafluoroethoxy, trifluoromethylthio, morpholin-4-ylcarbonyl, Anilinocarbonyl, methylcarbamoyl, tert-butylcarbamoyl, dimethylcarbamoyl, allylcarbamoyl, 3,3-dimethyl-2-oxobutyl, methylsulphonylamino, [methylaminosulphonyl]methyl, dimethylaminosulphonyl, [dimethylaminosulphonyl]oxy, acetamido, (2,2-dimethylpropanoyl)amino, formamido, formyl(methyl)amino, acetyl(methyl)amino, acetyl(ethyl)amino, acetyl, trifluoroacetyl, trifluoroacetylamino, 2-ethoxy-2-oxoethyl, cyanoacetyl, propionyl,
   where the other substituents have one or more of the meanings mentioned above,
   and also the agrochemically active salts thereof.

Preference is furthermore given to compounds of the formula (Ic) in which one or more of the symbols have one of the following meanings:
R² is hydrogen, fluorine, chlorine, bromine, iodine, cyano, nitro, hydroxyl, O—C₁-C₄-alkyl, O(CH₂)₂OCH₃, O(CH₂)₃OCH₃, O-cyclopentyl, OCF₃, OCF₂H, OCF₂CF₃, OCF₂CF₂H, O(CH₂)₂N(C₂H₅)₂, O(CH₂)₂N(CH₃)₂, OCH(CH₃)CH₂OCH₃, OSO₂NMe₂, OCONH(C₁-C₃-alkyl), OCON(C₁-C₃-alkyl)₂, OCO(C₁-C₄-alkyl), OSO₂N(CH₃)₂, SH, SF₅, S—C₁-C₃-alkyl, SCF₃, SCF₂H, SPh, SOMe, SONHMe, SONMe₂, SO₂Me, SO₂CF₃, SO₂CH₂CH═CH₂, SO₂CH₂CN, SO₂CH₂C≡CH, SO₂NH₂, SO₂NH(C₁-C₄-alkyl), SO₂N(C₁-C₄-alkyl)₂, SO₂NHAc, SO₂NHPh, SO₂NH(CH₂)₂N(CH₃)₂, SO₂NH(CH₂)₃N(CH₃)₂, SO₂NHCH₂CH═CH₂, CO(C₁-C₄-alkyl), COCHF₂, COCF₃, COCH₂CN, CONH(C₁-C₄-alkyl), CON(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, COCH₂NMe₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₃-alkoxy)carbonyl, CO₂(CH₂)₂OCH₃, NHCO(C₁-C₄-alkyl), N(C₂H₅)COMe, NHCOCH═CH₂, NHCOPh, NHCOCF₃, NHCOC(CH₃)₂CH₂F, NHCOC(CH₃)₂CH₂Cl, NHCO(C═CH₂)CH₃, NHCONMe₂, NHCOCH₂OCH₃, NHCO(CH₂)₂OCH₃, N(CH₃)COCH₃, N(C₂H₅)COCH₃, N(CH₃)COC(CH₃)₃, NHCHO, NMeCHO, NHCO₂(C₁-C₄-alkyl), NHCO₂Ph, NHCO₂CH₂CH₂Cl, NEtCO₂Me, NMeCO₂Me, NH(C═S)OMe, NH₂, NH(C₁-C₄-alkyl), N(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSOMe, NHSOCF₃, NHSO₂Me, NHSO₂CF₃, CH₂CN, CHMeCN, CH₂SO₂Me, CH₂SO₂NH(C₁-C₄-alkyl), CH₂COCH₃, CH₂COtertBu, CH(CH₃)COCH₃, CH₂COCH(CH₃)₂, CH₂CO-cyclopropyl, CH₂CONHtertBu, CH₂CO₂Et, (CH₂)₂OMe, (CH₂)₃OMe, C(CH₃)₂OCH₃, CH₂OisoPr, CH₂OtertBu, CH₂C(CH₃)₂OCH₃, CHCHF₂OH, CH₂OH, CH₂SMe, (CH₂)₂SMe, C(CH₃)₂SCH₃, CH₂NHCOO(C₁-C₄-alkyl), CH₂NHCOOBn, CH═NOMe, C(CH₃)═NOMe, CH═NOEt, C(CH₃)═NOEt, CH₂NH(CH₂)₂OCH₃, CH₂NAc₂, CH₂NHAc, CH₂NHCOCF₃, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, CH₂COOCH₃, CH₂COOEt, C₁-C₄-alkyl, C₃-C₆-cycloalkyl, 1-methoxycyclopropyl, 1-chlorocyclopropyl, 3,3-dimethylbutyl, cyclohexylmethyl, C₂-C₆-alkenyl, (trimethylsilyl)methyl, CF₃, CF₂H, CCl₃, C₂F₅, 4-(tert-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoylamino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methylcyclopropyl)carbonylamino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, (2,2,2-trifluoroethyl)carbamoyl, 5-ethoxy-3-(trifluoromethyl)-1H-pyrazol-1-yl, 3-(2-chloroethyl)-2-oxoimidazolidin-1-yl, 1-(methylsulphanyl)ethyl, 2-oxoazepan-1-yl, 2-oxopyridin-1(2H)-yl, 3-oxobutyl, acetyl(methoxy)amino, 1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
   and, if in each case two adjacent radicals R², R³ or R⁴, if appropriate via R¹² or R¹³, form a cycle, the following subunit from the general formula (I):

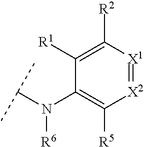

may be (2-oxo-2,3-dihydro-1H-indol-5-yl)amino, 1H-indol-6-ylamino, 1H-indol-5-yl amino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benz-imidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
   where the other substituents have one or more of the meanings mentioned above,
   and also the agrochemically active salts thereof.

Preference is furthermore given to compounds of the formula (Ic) in which one or more of the symbols have one of the following meanings:
R² is H, Cl, F, cyano, methyl, isopropyl, tert-butyl, propoxy, methoxy, methoxymethyl, ethoxycarbonyl, methylthio, ethylsulphanyl, methylsulphonyl, formamido, acetyl, 1,1,-dioxidoisothiazolidin-2-yl, 2-oxoazepan-1-yl, 2-oxopiperidin-1-yl, 2-oxopyrrolidin-1-yl, 1,3-thiazol-2-yl, 3-methyl-2-oxoimidazolidin-1-yl, 3-(2-chloroethyl)-2-oxoimidazolidin-1-yl, 3-oxomorpholin-4-yl, morpholin-4-ylsulphonyl, [(4-methylphenypamino]sulphonyl, aminosulphonyl, propionylamino, methacryloylamino, acryloylamino, (2,2-dimethylpropanoyl)amino, (methylsulphonyl)amino, (methylsulphonyl)methyl, piperidin-1-ylsulphonyl, [(dimethylamino)sulphonyl], [(dimethylamino)sulphonyl]oxy, acetamido, methoxycarbonylamino, ethoxycarbonylamino, 2-(tert-butylamino)-2-oxoethyl, dimethylcarbamoyl, (3-fluoro-2,2-dimethylpropanoyl)amino, 2-furoylamino, benzoylamino, 1,1,2,2-tetrafluoethoxy, cyclopropylcarbamoyl, (2,2,2-trifluoroethyl)carbamoyl, methylcarbamoyl, ethylcarbamoyl, diethylcarbamoyl, trifluoromethoxy, 2,2,2-trifluoro-1-hydroxyethyl, hydroxymethyl, cyanomethyl, propionyl, (methylcarbamoyl)oxy, (phenoxycarbonyl)amino, acetamido, 3-oxobutyl,
   where the other substituents have one or more of the meanings mentioned above,
   and also the agrochemically active salts thereof.

Preference is furthermore given to compounds of the formula (Ic) in which one or more of the symbols have one of the following meanings:
X¹ is CR³,
R² is H, Cl, F, cyano, methyl, isopropyl, tert-butyl, propoxy, methoxy, methoxymethyl, ethoxycarbonyl, methylthio, ethylsulphanyl, methylsulphonyl, formamido, acetyl, 1,1,-dioxidoisothiazolidin-2-yl, 2-oxoazepan-1-yl, 2-oxopiperidin-1-yl, 2-oxopyrrolidin-1-yl, 1,3-thiazol-2-yl, 3-methyl-2-oxoimidazolidin-1-yl, 3-(2-chloroethyl)-2-oxoimidazolidin-1-yl, 3-oxomorpholin-4-yl, morpholin-4-ylsulphonyl, [(4-methylphenypamino]sulphonyl, aminosulphonyl, propionylamino, methacryloylamino, acryloylamino, (2,2-dimethylpropanoyl)amino, (methylsulphonyl)amino, (methylsulphonyl)methyl, piperidin-1-ylsulphonyl, [(dimethylamino)sulphonyl], [(dimethylamino)sulphonyl]oxy, acetamido, methoxycarbonylamino, ethoxycarbonylamino, 2-(tert-butylamino)-2-oxoethyl, dimethylcarbamoyl, (3-fluoro-2,2-dimethylpropanoyl)amino, 2-furoylamino, benzoylamino, 1,1,2,2-tetrafluoethoxy, cyclopropylcarbamoyl, (2,2,2-trifluoroethyl)carbamoyl, methylcarbamoyl, ethylcarbamoyl, diethylcarbamoyl, trifluoromethoxy, 2,2,2-trifluoro-1-hydroxyethyl, hydroxymethyl, cyanomethyl, propionyl, (methylcarbamoyl)oxy, (phenoxycarbonyl)amino, acetamido, 3-oxobutyl,
R³ is H, halogen, CN, COOH, methoxycarbonyl, pentanoyloxy, OH, methoxy, ethoxy, isopropoxy, methylthio, difluoromethylthio, methylsulphonyl, trifluoromethylsulphonyl, methylamino, methyl, isopropyl, 2-methoxy-1-methylethoxy, acetyl(cyclopropyl)amino, acetyl(cyclohexyl)amino, (1-methylcyclopropyl)carbonylamino, methoxycarbonylamino, (ethoxycarbonyl)amino, (tert-butoxycarbonyl)amino, [(2-chloroethoxy)carbonyl]-amino, (3-chloro-2,2-dimethylpropanoyl)amino, (3-fluoro-2,2-dimethylpropanoyl)amino, isobutyrylamino, 3-methyl,-2,5-dioxoimidazolidin-1-yl, 2,5-dioxoimidazolidin-4-yl, 3-oxomorpholin-4-yl, 2-oxo-1,3-oxazolidin-3-yl, 2-oxopyrrolidin-1-yl, 2,5-dioxopyrrolidin-1-yl, 1,3-thiazol-4-yl, CF₃, difluoromethoxy, trifluoromethoxy, 1,1,2,2-tetrafluoroethoxy, trifluoromethylthio, morpholin-4-ylcarbonyl, anilinocarbonyl, methylcarbamoyl, tert-butylcarbamoyl, dimethylcarbamoyl, allylcarbamoyl, 3,3-dimethyl-2-oxobutyl, methylsulphonylamino, [methylaminosulphonyl]methyl, dimethylaminosulphonyl, [dimethylaminosulphonyl]oxy, acetamido, (2,2-dimethylpropanoyl)amino, formamido, formyl(methyl)amino, acetyl(methyl)amino, acetyl(ethyl)amino, acetyl, trifluoroacetyl, trifluoroacetylamino, 2-ethoxy-2-oxoethyl, cyanoacetyl, propionyl,
   where the other substituents have one or more of the meanings mentioned above,
   and also the agrochemically active salts thereof.

The radical definitions mentioned above can be combined with one another as desired. Moreover, individual definitions may not apply.

The compounds of the formulae (I), (Ia), (Ib) and (Ic) can be present both in pure form and as mixtures of various possible isomeric forms, in particular of stereoisomers, such as E and Z, threo and erythro, and also optical isomers, such as R and S isomers or atropisomers, and, if appropriate, also of tautomers. What is claimed are both the E and the Z isomers, and also the threo and erythro, and also the optical isomers, any mixtures of these isomers, and also the possible tautomeric forms.

Depending on the nature of the substituents defined above, the compounds of the formulae (I), (Ia), (Ib) and (Ic) have acidic or basic properties and may form salts with inorganic or organic acids or with bases or with metal salts, if appropriate also inner salts or adducts. If the compounds of the formulae (I), (Ia), (Ib) and (Ic) carry amino, alkylamino or other groups which induce basic properties, these compounds can be reacted with acids to salts or are obtained directly as salts in the synthesis. If the compounds of the formulae (I), (Ia), (Ib) and (Ic) carry hydroxyl, carboxy or other groups which induce acidic properties, these compounds can be reacted with bases to salts. Suitable bases are, for example, hydroxides, carbonates, bicarbonates of the alkali metals and the alkaline earth metals, in particular those of sodium, potassium, magnesium and calcium, furthermore ammonia, primary, secondary and tertiary amines having (C₁-C₄-)-alkyl groups, mono-, di- and trialkanolamines of (C₁-C₄)-alkanols, choline and also chlorocholine.

The salts obtainable in this manner also have fungicidal, herbicidal and insecticidal properties.

Examples of inorganic acids are hydrohalic acids such as hydrogen fluoride, hydrogen chloride, hydrogen bromide and hydrogen iodide, sulphuric acid, phosphoric acid and nitric acid and acidic salts such as NaHSO₄ and KHSO₄. Suitable organic acids are, for example, formic acid, carbonic acid and alkanoic acids such as acetic acid, trifluoroacetic acid, trichloroacetic acid and propionic acid and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or -disulphonic acids (aromatic radicals such as phenyl and naphthyl which carry one or two sulphonic acid groups), alkylphosphonic acids (phosphonic acids having straight-chain or branched alkyl radicals of 1 to 20 carbon atoms), arylphosphonic acids or -diphosphonic acids (aromatic radicals such as phenyl and naphthyl which carry one or two phosphonic acid groups), where the alkyl or aryl radicals may carry further substituents, for example, p-toluenesulphonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals may be present in the various valencies that they can assume.

Optionally substituted groups can be mono- or polysubstituted, where in the case of polysubstitution the substituents can be identical or different.

In the definitions of the symbols given in the formulae above, collective terms were used that are generally representative for the following substituents:

halogen: fluorine, chlorine, bromine and iodine;
alkyl: saturated straight-chain or branched hydrocarbon radicals having 1 to 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 8 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 8 carbon atoms and a double bond in any position, for example C₂-C₆-alkenyl, such as ethenyl, 1-propenyl, 2-prop enyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1,-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated, straight-chain or branched alkoxy radicals having 1 to 8 carbon atoms, for example C₁-C₆-alkoxy, such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethyl-butoxy, 3,3-dimethylbutoxy, 1-ethylbutoxy, 2-ethylbutoxy, 1,1,2-trimethylpropoxy, 1,2,2-tri-methylpropoxy, 1-ethyl-1-methylpropoxy and 1-ethyl-2-methylpropoxy;
haloalkoxy: straight-chain or branched alkoxy groups having 1 to 8 carbon atoms (as mentioned above), where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2-difluoroethoxy, 2,2-dichloro-2-fluoroethoxy, 2,2,2-trichloroethoxy, pentafluoroethoxy and 1,1,1-trifluoroprop-2-oxy;
alkylamino: saturated, straight-chain or branched alkylamino radicals having 1 to 8 carbon atoms, for example C₁-C₆-alkylamino, such as methylamino, ethylamino, propylamino, 1-methylethylamino, butylamino, 1-methylpropylamino, 2-methylpropylamino, 1,1-dimethyl-ethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethyl-propylamino, 1-methylpentylamino, 2-methylpentylamino, 3-methylpentylamino, 4-methyl-pentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino and 1-ethyl-2-methylpropylamino;
alkylthio: saturated, straight-chain or branched alkylthio radicals having 1 to 8 carbon atoms, for example C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methyl-pentylthio, 4-methylpentylthio, 1,1-dimethylbutylthio, 1,2-dimethylbutylthio, 1,3-dimethyl-butylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
haloalkylthio: straight-chain or branched alkylthio groups having 1 to 8 carbon atoms (as mentioned above), where some or all of the hydrogen atoms in these groups may be replaced by halogen atoms as mentioned above, for example C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;
alkylsulphinyl: saturated, straight-chain or branched alkylsulphinyl radicals having 1 to 8 carbon atoms, for example C₁-C₆-alkylsulphinyl, such as methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethyl-butylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethyl-propylsulphinyl, 1-ethyl-1-methylpropylsulphinyl and 1-ethyl-2-methylpropylsulphinyl;
alkylsulphonyl: saturated, straight-chain or branched alkylsulphonyl radicals having 1 to 8 carbon atoms, for example C₁-C₆-alkylsulphonyl, such as methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl;
cycloalkyl: monocyclic saturated hydrocarbon groups having 3 to 8 carbon ring members, such as cyclopropyl, cyclobutyl, cyclopentyl and cyclohexyl;
cylcoalkenyl: monocyclic nonaromatic hydrocarbon groups having 3 to 8 carbon ring members and at least one double bond, such as cyclopenten-1-yl, cyclohexen-1-yl, cyclohepta-1,3-dien-1-yl;
alkoxycarbonyl: an alkoxy group having 1 to 6 carbon atoms (as mentioned above) which is attached to the skeleton via a carbonyl group (—CO—);
oxyalkylenoxy: divalent unbranched chains of 1 to 3 CH₂ groups where both valencies are attached to the skeleton via an oxygen atom, for example, OCH₂O, OCH₂CH₂O and OCH₂CH₂CH₂O;
a three- to ten-membered saturated or partially unsaturated heterocycle which contains one to four heteroatoms from the group consisting of oxygen, nitrogen and sulphur: mono- or bicyclic heterocycles (heterocyclyl) which contain, in addition to carbon ring members, one to three nitrogen atoms and/or one oxygen or sulphur atom or one or two oxygen and/or sulphur atoms; if the ring contains a plurality of oxygen atoms, these are not directly adjacent; for example, oxiranyl, aziridinyl, 2-tetrahydrofuranyl, 3-tetrahydrofuranyl, 2-tetrahydrothienyl, 3-tetra-hydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydroopyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetra-hydropyranyl, 2-tetrahydrothienyl, 3-hexahydropyridazinyl, 4-hexahydropyridazinyl, 2-hexa-hydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
a five- to ten-membered aromatic heterocycle which contains one to four heteroatoms from the group consisting of oxygen, nitrogen and sulphur: mono- or bicyclic heteroaryl, for example,
5-membered heteroaryl which contains one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members and in which two adjacent carbon ring members or one nitrogen and an adjacent carbon ring member may be bridged by a buta-1,3-diene-1,4-diyl group in which one or two carbon atoms may be replaced by nitrogen atoms;
5-membered heteroaryl which is attached via nitrogen and contains one to four nitrogen atoms, or benzo-fused 5-membered heteroaryl which is attached via nitrogen and contains one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and an adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
   6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
   leaving group: an S_{N}1 or S_{N}2 leaving group, for example halogen (chlorine, bromine, iodine), alkylsulphonate (—OSO₂-alkyl, for example —OSO₂CH₃, —OSO₂CF₃) or arylsulphonate (—OSO₂-aryl, for example —OSO₂Ph, —OSO₂PhMe);

Not included are combinations which are contrary to natural laws and which the person skilled in the art would therefore have excluded based on his or her expert knowledge. Ring structures having three or more adjacent oxygen atoms, for example, are excluded.

The invention furthermore provides a process for preparing the diaminopyrimidines of the formulae (Ia), (Ib) and (Ic) according to the invention, which process comprises at least one of steps (a) to (e) below:
(a) the reaction of 2,4-dihalopyrimidines of the formula (III) in the presence of a base, if appropriate in the presence of a solvent, if appropriate in the presence of a catalyst with cyclobutylamines of the formula (II) to give compounds of the formula (V), according to the reaction scheme below (Scheme 1):

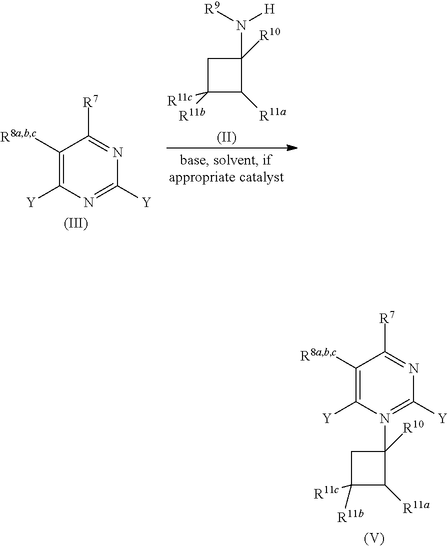

where Y═F, Cl, Br, I
(b) the reaction of compounds of the formula (V), if appropriate in the presence of an acid, if appropriate in the presence of a solvent, with aromatic amines of the formula (IV), according to the reaction scheme below (Scheme 2):

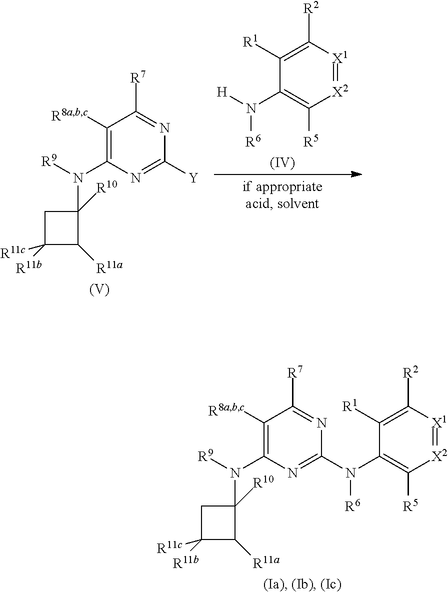

where Y═F, Cl, Br, I
(c) the reaction of compounds of the formula (VIb), if appropriate in the presence of an acid, if appropriate in the presence of a solvent, with an aromatic amine of the formula (IV), according to the reaction scheme below (Scheme 3):

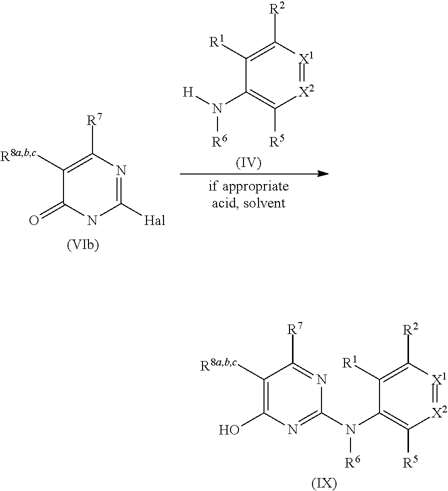

where Hal=F, Cl, Br, I
(d) the reaction of compounds of the formula (IX), if appropriate in the presence of a solvent, with a halogenating agent to give compounds of the formula (X), according to the reaction scheme below (Scheme 4):

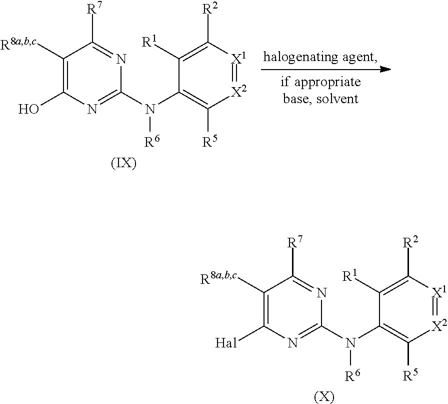

(e) the reaction of compounds of the formula (X) in the presence of a base, if appropriate in the presence of a solvent, if appropriate in the presence of a catalyst with cyclobutylamines of the formula (II) to give compounds of the formulae (Ia, Ib and Ic), according to the reaction scheme below (Scheme 5):

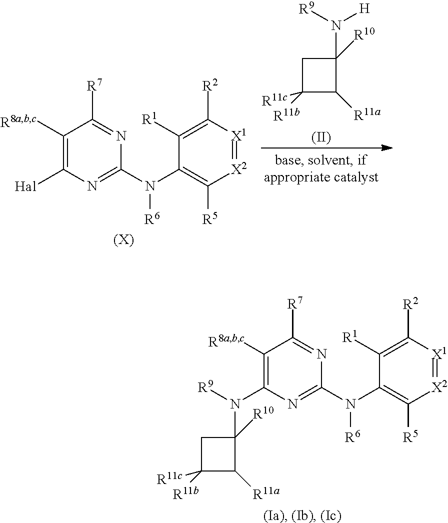

where the definitions of the radicals R¹ to R^{11c} and X¹ and X² in the above schemes correspond to the definitions given above, and Y and Hal represent F, Cl, Br, I.

One way of preparing the intermediate (V) is shown in Scheme 1.

The amino compounds of the formula (II) are either commercially available, or they can be prepared according to procedures from the literature. One method of preparing suitable amino verbindungen (II) is, for example, the rearrangement of suitable carboxylic acid derivatives to the corresponding amino compounds (described, for example, in J. Am. Chem. Soc. 1961, 83, 3671-3678). Further methods for preparing amines (II) comprise, for example, the hydroboration of suitable cyclobutenes and subsequent treatment with NH₂SO₃H (for example Tetrahedron 1970, 26, 5033-5039), the reductive amination of cyclobutanones (described, for example, in J. Org. Chem. 1964, 29, 2588-2592) and also the reduction of nitro- or nitrosocyclobutanes (see, for example, J. Am. Chem. Soc. 1953, 75, 4044; Can. J. Chem. 1963, 41, 863-875) or azidocyclobutanes (described, for example, in Chem. Pharm. Bull. 1990, 38, 2719-2725; J. Org. Chem. 1962, 27, 1647-1650).

Suitable substituted 2,4-dihalopyrimidines (III) are either commercially available or can be prepared according to procedures from the literature, for example from commercially available substituted uracils (for example R⁸═CN: J. Org. Chem. 1962, 27, 2264; J. Chem. Soc. 1955, 1834; Chem. Ber. 1909, 42, 734; R⁸═CF₃: J. Fluorine Chem. 1996, 77, 93; see also WO 2000/047539).

Initially, using a suitable base at a temperature of from −30° C. to +80° C. in a suitable solvent such as, for example, dioxane, THF, dimethylformamide or acetonitrile, a cyclobutylamine (II) is reacted with a 2,4-dihalopyrimidine (III) over a period of 1-24 h. Suitable for use as bases are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst such as, for example, palladium together with a suitable ligand such as, for example, triphenylphosphine or xanthphos.

Some of the compounds of the formula (V) are novel and therefore also form part of the subject matter of the present invention.

Novel are compounds of the formula (Va) in which

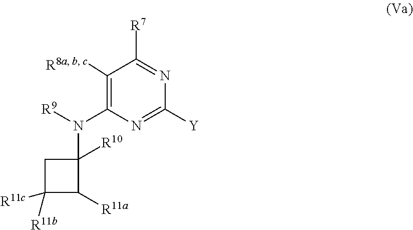

R^{8a} represents iodine, CFH₂, CF₂H, CCl₃, cyano or Me,
R^{8b} represents CF₃;
R^{8C} represents Br
Y═F, Cl, Br or I
   and
R⁷, R⁹, R¹⁰, R^{11a}, R^{11b} and R^{11c} have the general, preferred, particularly preferred and very particularly preferred meanings defined above.

One way of preparing the compounds (Ia), (Ib) and (Ic) is shown in Scheme 2.

The substituted (het-)aromatic amines (IV) are either commercially available or can be prepared by methods known from the literature from commercially available precursors. Aromatic amines which carry one or more identical or different substituents in the aromatic moiety can be prepared by a large number of methods described in the relevant literature. Some of the methods are mentioned below by way of example.

Sulphonamide- or sulphonic ester-substituted (het-)arylamines can be prepared, for example, by the reaction, known from the literature, of commercially available aminosulphonic acids with chlorinating agents (for example POCl₃) and subsequent reaction of the sulphochlorides formed with O- or N-nucleophiles.

Two frequently used methods for preparing N-monoacylated diamino(het-)aromatics are illustrated below. Thus, for example, nitroanilines can be reacted by standard methods with acyl halides, chloroformates or iso(thio)cyanates to give the corresponding N-acylnitroaromatic which can then be reduced according to procedures known from the literature to give N-acyl-aminoaromatics. A further method describes the preparation of the compounds mentioned by transition metal-catalyzed cross-coupling of aminohaloaromatic and N-acyl compounds (see, for example, J. Am. Chem. Soc. 2001, 123, 7727).

### Synthesis of N-Monoacylated Diaminoaromatics

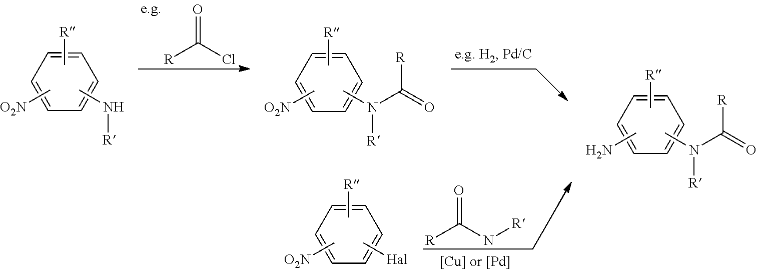

Cyclic radicals R¹ to R⁵ attached via nitrogen can be prepared, for example, by condensation of nitroaminoaromatics with haloalkylcarbonyl halides or diesters or diester equivalents or lactones; the subsequent reduction of the nitro group affords the desired aromatic amine. A further way of synthesizing radicals R¹ to R⁵ attached via nitrogen is the condensation of nitroarylhydrazines with diesters or diester equivalents, propargyl acid esters or keto esters. The reduction of the nitro group gives the aniline.

The intermediate (V) is, in the presence of Brönstedt acids such as, for example, anhydrous hydrochlorid acid, camphorsulphonic acid or p-toluenesulphonic acid, reacted in a suitable solvent such as, for example, dioxane, THF, DMSO, DME, 2-methoxyethanol, n-butanol or acetonitrile at a temperature of 0° C.-140° C. over a period of 1-48 h with a (het-)aromatic amine (IV). Analogously described, for example, in Bioorg. Med. Chem. Lett. 2006, 16, 2689; GB2002 A1-2369359, Org. Lett. 2005, 7, 4113.

Alternatively, the reaction of (V) and (IV) to give (Ia), (Ib) and (Ic) can also be carried out with base catalysis, that is using, for example, carbonates, such as potassium carbonate, alkoxides, such as potassium tert-butoxide, or hydrides, such as sodium hydride, where the catalytic use of a transition metal such as, for example, palladium together with a suitable ligand such as, for example, xanthphos may also be of use.

Finally, it is possible to carry out the reaction of (V) and (IV) to give (I) in the absence of solvents and/or Brönstedt acids (described, for example, in Bioorg. Med. Chem. Lett. 2006, 16, 108; Bioorg. Med. Chem. Lett. 2005, 15, 3881).

One way of preparing compounds of the formula (IX) and also (IXa) is shown in Scheme 3.

2-Halo-substituted pyrimidin-4-ones (VIb) are obtainable from 2,4-dihalo-substituted pyrimidines by regioselective hydrolysis. This is described, for example, in Russ. J. Org. Chem. 2006, 42, 580; J. Med. Chem. 1965, 8, 253.

Intermediates of the formula (VIb) are reacted in the presence of Brönstedt acids such as, for example, anhydrous hydrochloric acid, camphorsulphonic acid or p-toluenesulphonic acid in a suitable solvent such as, for example, dioxane, THF, DMSO, DME, 2-methoxyethanol, n-butanol or acetonitrile at a temperature of 0° C.-140° C. over a period of 1-48 h with a (het-)aromatic amine (IV).

Alternatively, the reaction of (VIb) and (IV) to give (IX) can also be carried out with base catalysis, that is using, for example, carbonates, such as potassium carbonate, alkoxides, such as potassium tert-butoxide, or hydrides, such as sodium hydride, where the catalytic use of a transition metal such as, for example, palladium together with a suitable ligand such as, for example, xanthphos may also be of use.

Finally, it is possible to carry out the reaction of (VIb) and (IV) to give (IX) in the absence of solvents and/or Brönstedt acids (described, for example, in Bioorg. Med. Chem. Lett. 2006, 16, 108; Bioorg. Med. Chem. Lett. 2005, 15, 3881).

Some of the compounds of the formula (IX) are novel and therefore also form part of the subject matter of the present invention.

Novel are compounds of the formula (IXa),

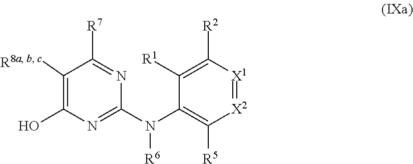

in which the symbols have the following meanings:
X¹, X², R¹ to R⁵, R^{8b}, R^{8c}, R¹² and R¹³ have the general, preferred, particularly preferred, very particularly preferred and especially preferred meanings indicated above,
R⁶ and R⁷ are hydrogen.
R^{8a} represents chlorine, iodine, CFH₂, CF₂H, CCl₃
   with the proviso that, if
R¹═R²═R⁵═H and X²═CH or N,
R³ must not be H, CO₂H, (CH₂)₂OH, SMe, SOMe, SO₂NH₂ or cyano
   or
   with the proviso that, if
R¹═R⁵═H and X¹═CH,
   neither R² nor R⁴ must be OH or CONH₂.

One way of preparing compounds of the formula (X) and also (Xa) is shown in Scheme 4.

Intermediates of the formula (IX) can be converted by reaction with suitable halogenating agents such as, for example, thionyl chloride, phosphorus pentoxide or phosphoryl chloride or a mixture thereof; if appropriate in the presence of a suitable solvent such as, for example, toluene or ethanol and, if appropriate, in the presence of a suitable base such as, for example, triethylamine, into 2-anilino-4-chloropyrimidines of the formula (X). Analogously described, for example, in J. Med. Chem. 1989, 32, 1667; J. Heterocycl. Chem. 1989, 26, 313.

Some of the compounds of the formula (X) are novel and therefore also form part of the subject matter of the present invention.

Novel are compounds of the formula (Xa),

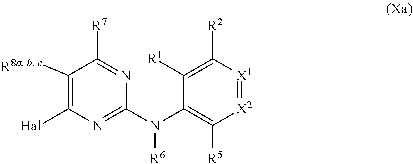

in which the symbols have the following meanings:
X¹, X², R² to R⁴, R⁷, R^{8b}, R^{8c}, R¹² and R¹³ have the general, preferred, particularly preferred, very particularly preferred and especially preferred meanings indicated above and
Hal represents fluorine, chlorine, bromine or iodine,
R^{8a} represents chlorine, iodine, CFH₂, CF₂H, CCl₃ and cyano,
R¹, R⁵ and R⁶ represent hydrogen
   with the proviso that, if
X²═CH or N and X¹═CR³,
R³ is not CON(Me)-4-(N-methylpiperidinyl), N-piperazinyl, CO-1-(4-methylpiperazinyl), N-morpholinyl, SO₂Me, CONH₂, Me, OMe, COO-benzyl, COOH, COCl, CN, SO₂NH₂, NO₂, NMe₂ or Cl,
   or
   with the proviso that, if
X¹═CH and X²═CR⁴,
R² or R⁴ does not represent CN, Cl or 5-oxazolyl,
   or
   with the proviso that, if
X¹═CR³ and X²═CR⁴,
R², R³ and R⁴ are not chlorine,
   or
   with the proviso that, if
X¹═CR³, X²═CR⁴ and R^{8b}═CF₃,
R² and R³ or R³ and R⁴ together do not form a saturated or partially unsaturated heterocycle.

A further way of preparing the compounds (Ia), (Ib), and (Ic) is shown in Scheme 5.

To prepare compounds of the formulae (Ia), (Ib) and (Ic), the intermediate (X) is, in the presence of bases such as, for example, carbonates such as potassium carbonate, alkoxides such as potassium tert-butoxide or hydrides such as sodium hydride, reacted in a suitable solvent such as, for example, dioxane, THF, DMSO, DME, 2-methoxyethanol, n-butanol or acetonitrile at a temperature of 0° C.-140° C. over a period of 1-48 h with cyclobutylamines of the formula (II), where the catalytic use of a transition metal such as, for example, palladium together with a suitable ligand such as, for example, triphenylphosphine or xanthphos may also be of use.

In general, it is also possible to chose another way to prepare the compounds (Ia), (Ib) and (Ic) according to the invention, as shown in Scheme 6.

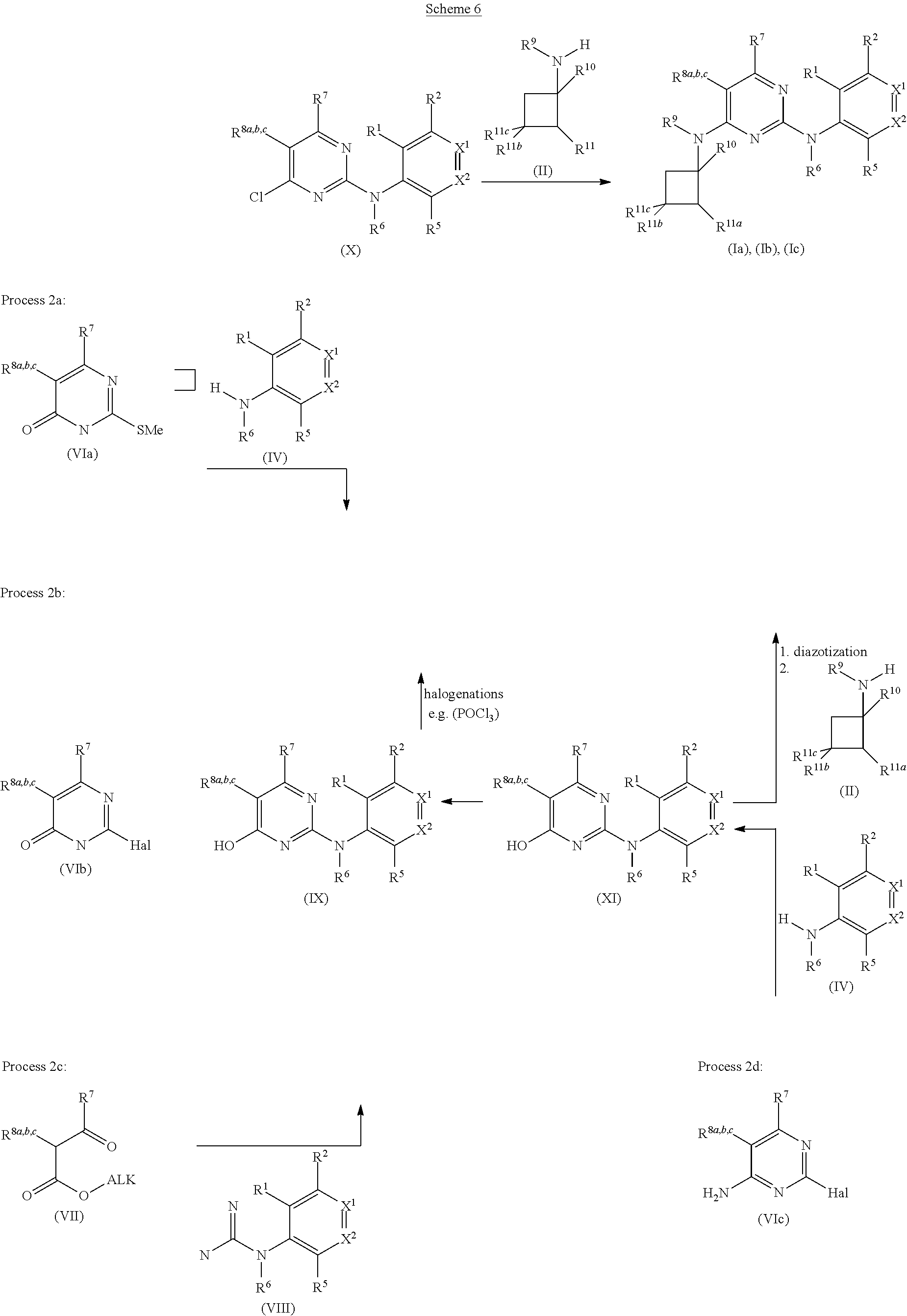

A further way of preparing diaminopyrimidines of the formulae (Ia), (Ib) and (Ic) is shown in Scheme 7:

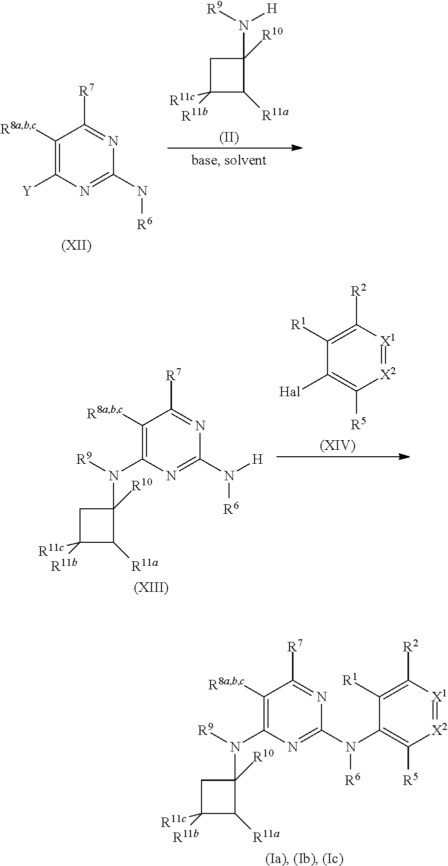

Starting with 4-halo-substituted 2-aminopyrimdines (XII), which can be prepared, for example, analogously to (X) from compounds of the type (VIa), (VIb) or (VII) by reaction with R⁶-amines with subsequent chlorination in the 4-position, following addition of an amino compound (II) it is possible to obtain certain diaminopyrimidines (XIII) In a subsequent transition metal-catalysed step, these can be reacted with an aryl halide (XIV) (as described, for example, in Org. Lett. 2002, 4, 3481) to give the desired target compound (Ia), (Ib), (Ic).

The processes according to the invention for preparing the compounds of the formula (Ia), (Ib) and (Ic) are preferably carried out using one or more reaction auxiliaries.

Suitable reaction auxiliaries are, if appropriate, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or isopropoxide, n-, iso-, s- or t-butoxide or potassium methoxide, ethoxide, n- or isopropoxide, n-, iso-, s- or t-butoxide; furthermore also basic organic nitrogen compounds such as, for example, trimethylamine, triethylamine, tripropylamine, tributyl-amine, ethyldiisopropylamine, N,N-dimethylcyclohexylamine, dicyclohexylamine, ethyldicyclo-hexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo [4.3.0]non-5-ene (DBN) or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and di-oxane, ketones, such as acetone, methylethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethylsulphoxide, tetramethylene sulphone and hexamethyl-phosphoric triamide and DMPU.

The reaction temperatures in the processes according to the invention can be varied within a relatively wide range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use a relatively large excess of in each case one of the components employed. In the processes according to the invention, work-up is in each case carried out by customary methods (cf. the Preparation Examples).

In general, compounds of the formula (I) can be prepared, for example, by sequential nucleophilic addition of an alicyclic amine (II) and a (hetero)aromatic amine (IV) to a suitable substituted pyrimidine (III), as illustrated below in Scheme 8:

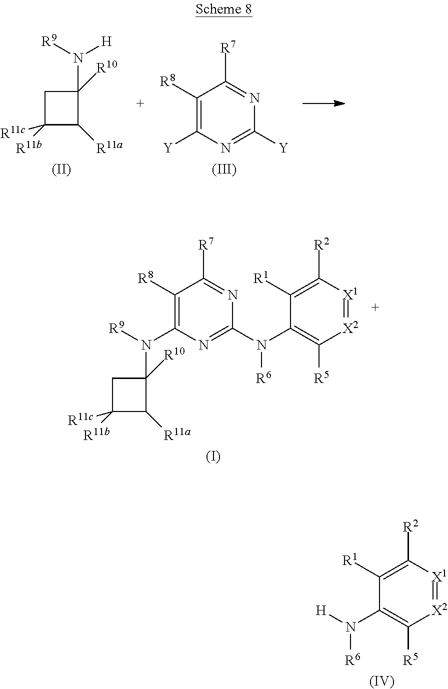

Here, the radicals Y are each independently of one another suitable leaving groups, for example halogen atoms (Hal=F, Cl, Br, I), SMe, SO₂Me, SOMe or else triflate (CF₃SO₂O: in the case of pyrimidines known from WO2005095386).

The synthesis of diaminopyrimidines of the formula (I) according to Scheme 8 or else by different routes has been described numerous times in the literature (see also, for example, WO 2006/021544, WO 2007/072158, WO 2007/003596, WO 2005/016893, WO 05/013996, WO 04/056807, WO 04/014382, WO 03/030909)

The invention furthermore relates to the non-medical use of the diaminopyrimidines according to the invention or mixtures thereof for controlling unwanted microorganisms, in particular phytopathogenic harmful fungi, insects or weeds.

The invention furthermore relates to a composition for controlling unwanted microorganisms, in particular phytopathogenic harmful fungi, insects or weeds, which composition comprises at least one diaminopyrimidine according to the present invention.

Moreover, the invention relates to a method for controlling unwanted microorganisms, in particular phytopathogenic harmful fungi, insects or weeds, which method is characterized in that the diaminopyrimidines according to the invention are applied to the microorganisms, in particular phytopathogenic harmful fungi, insects or weeds and/or to their habitat.

The invention furthermore relates to seed treated with at least one diaminopyrimidine according to the invention.

Finally, the invention relates to a method for protecting seed against unwanted microorganisms by using seed treated with at least one diaminopyrimidine according to the present invention.

The compounds according to the invention have strong microbicidal action and can be used for controlling unwanted microorganisms, such as fungi and bacteria, in crop protection and in the protection of materials.

The diaminopyrimidines of the formulae (I), (Ia), (Ib and (Ic) according to the invention have very good fungicidal properties and can be used in crop protection, for example, for controlling Plasmodiophoromycetes, Oomycetes, Chytridiomycetes, Zygomycetes, Ascomycetes, Basidiomycetes and Deuteromycetes

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil in which the plants grow.

The compositions according to the invention for controlling phytopathogenic fungi in crop protection comprise an effective, but not phytotoxic, amount of the active compounds according to the invention. “Effective, but not phytotoxic, amount” means an amount of the composition according to the invention which is sufficient to control the fungal disease of the plant sufficiently or even to kill it completely and which at the same time does not cause any phytotoxicity symptoms worth mentioning. In general, this application rate may vary within a relatively wide range. It depends on a number of factors, for example on the fungus to be controlled, on the plant, on the climatic conditions and on the ingredients of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetable, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such as Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussel sprouts, pak choi, kohlrabi, garden radish, and also oilseed rape, mustard, horseradish and cress), Fabacae sp. (for example beans, peas), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collocygni; Rhynchosporium species, such as, for example, Rhynchosporium secalis; Septoria species, such as, for example, Septoria apii; Typhula species, such as, for example, Typhula incamata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;

Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia Southern blight (Sclerotinia rolfsii), thielaviopsis root rot (Thielaviopsis basicola).

The active compounds according to the invention also have very good fortifying action in plants. Accordingly, they can be used for mobilizing the defenses of the plant against attack unwanted microorganisms.

In the present context, plant-fortifying (resistance-inducing) substances are to be understood as meaning those substances which are capable of stimulating the defense system of plants such that, when the treated plants are subsequently inoculated with unwanted microorganisms, they show substantial resistance against these microorganisms.

In the present case, unwanted microorganisms are to be understood as meaning phytopathogenic fungi and bacteria. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

Here, the active compounds according to the invention can be used with particularly good results for controlling cereal diseases such as, for example, against Erysiphe species, against Puccinia and against Fusarien species, rice diseases such as, for example, against Pyricularia and Rhizoctonia and diseases in viticulture and the cultivation of fruit and vegetables such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

If appropriate, the compounds according to the invention can, at certain concentrations or application rates, also be used as herbicides, for regulating plant growth and for controlling animal pests. If appropriate, they can also be employed as intermediates or precursors for the synthesis of other active compounds.

The active compounds according to the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may preferably be employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp. Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the Helminthen, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vet micularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitell les spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculate.

From the order of the Thysanoptera, for example, Baliothrips biformis, Enneothrips Havens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

The compounds of the formula (I) according to the invention are distinguished in particular by strong activity against insects, parasites from the sub-class of the Acari (Acarina) (such as mites, spider mites and/or ticks) and/or nematodes.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc. It is furthermore possible to apply the active compounds by the ultra-low volume method or to inject the active compound preparation or the active compound itself into the soil.

The active compounds according to the invention can also be used as defoliants, desiccants, haulm killers and, in particular, as weed killers. Weeds in the broadest sense are understood as meaning all plants which grow at locations where they are undesired. Whether the substances according to the invention act as nonselective or selective herbicides depends essentially on the application rate.

The active compounds according to the invention can be used, for example, in the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but extends in the same manner to other plants.

Depending on the concentration, the active compounds according to the invention are suitable for the nonselective weed control on, for example, industrial terrains and railway tracks and on paths and locations with and without trees. Likewise the active compounds according to the invention can be employed for controlling weeds in perennial crops, for example forests, ornamental tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The active compounds according to the invention have strong herbicidal activity and a broad activity spectrum when used on the soil and on aerial plant parts. To a certain extent, they are also suitable for the selective control of monocotyledonous and dicotyledonous weeds in monocotyledonous and dicotyledonous crops, both pre- and post-emergence.

The active compounds or active compound combinations according to the invention can be applied both before and after plant emergence. They can also be incorporated into the soil prior to planting.

The application rate of active compound can vary within a substantial range. Essentially, it depends on the nature of the desired effect. In general, the application rates are between 1 g and 10 kg of active compound per hectare of soil area, preferably between 5 g and 5 kg per ha.

The advantageous effect of the compatibility with crop plants of the active compound combinations according to the invention is particularly pronounced at certain concentration ratios. However, the weight ratios of the active compounds in the active compound combinations can be varied within relatively wide ranges. In general, from 0.001 to 1000 parts by weight, preferably from 0.01 to 100 parts by weight, particularly preferably 0.05 to 20 parts by weight, of one of the compounds which improves crop plant compatibility (antidotes/safeners) mentioned above under (b′) are present per part by weight of active compound of the formula (I).

The active compound combinations according to the invention are generally applied in the form of finished formulations. However, the active compounds contained in the active compound combinations can, as individual formulations, also be mixed during use, i.e. be applied in the form of tank mixes.

For certain applications, in particular by the post-emergence method, it may furthermore be advantageous to include, as further additives in the formulations, mineral or vegetable oils which are tolerated by plants (for example the commercial preparation “Rako Binol”), or ammonium salts, such as, for example, ammonium sulphate or ammonium thiocyanate.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

Moreover, in the protection of materials, the active compounds or compositions according to the invention can be employed for protecting industrial materials against attack and destruction by unwanted microorganisms, such as, for example, fungi.

In the present context, industrial materials are understood as meaning non live materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example, cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The active compounds or compositions according to the invention may prevent disadvantageous effects, such as rotting, decay, discolouration, decolouration or formation of mould.

The method according to the invention for controlling unwanted fungi can also be employed for protecting storage goods. Here, storage goods are to be understood as meaning natural substances of vegetable or animal origin or processed products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound according to the invention can prevent disadvantageous effects, such as rotting, decay, discolouration, decolouration or the formation of mould.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and against slime organisms and algae. Examples which may be mentioned are microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms, which composition comprises at least one of the diaminopyrimidines according to the invention. These are preferably fungicidal composition which comprise agriculturally suitable auxiliaries, solvents, carriers, surfactants or extenders.

According to the invention a carrier is a natural or synthetic organic or inorganic substance which is mixed with or associated with the active compounds for better applicability, especially for application to plants or plant parts or seed. The carrier, which may be solid or liquid, is generally inert and should be suitable for use in agriculture.

Suitable solid carriers are: for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkylsulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspoemulsion concentrates, natural materials impregnated with active compound, synthetic materials impregnated with active compound, fertilizers and microencapsulations in polymeric substances.

The active compounds can be applied as such, in the form of their formulations or the use foams prepared therefrom, such as ready-to-use solutions, emulsions, water- or oil-based suspensions, powders, wettable powders, pastes, soluble powders, dusts, soluble granules, granules for broadcasting, suspoemulsion concentrates, natural products impregnated with active compound, synthetic materials impregnated with active compound, fertilizers and also microencapsulations in polymeric substances. Application is carried out in a customary manner, for example by watering, spraying, atomizing, broadcasting, dusting, foaming, spreading, etc. It is further possible to apply the active compounds by the ultra-low-volume method, or to inject the active compound preparation or the active compound itself into the soil. It is also possible to treat the seed of the plants.

The formulations mentioned can be prepared in a manner known per se, for example by mixing the active compounds with at least one customary extender, solvent or diluent, emulsifier, dispersant, and/or binder or fixative, wetting agent, water-repellent, if appropriate dessicants and UV stabilizers and, if appropriate, dyes and pigments, defoamers, preservatives, secondary thickeners, adhesives, gibberellins and also further processing auxiliaries.

The compositions according to the invention include not only formulations which are already ready for use and can be applied with a suitable apparatus to the plant or the seed, but also commercial concentrates which have to be diluted with water prior to use.

The active compounds according to the invention can be present as such or in their (commercial) formulations and in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethylsulphoxide).

Liquefied gaseous extenders or carriers refer to liquids which are gaseous at standard temperature and standard pressure, for example aerosol propellants, such as halogenated hydrocarbons, and also butane, propane, nitrogen and carbon dioxide.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and also their ethers and esters, ketones such as acetone, methylethyl ketone, methyl isobutyl ketone or cyclohexanone, strongly polar solvents such as dimethylformamide and dimethylsulphoxide, and also water.

The compositions according to the invention may comprise additional further components, such as, for example, surfactants. Suitable surfactants are emulsifiers and/or foam formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkylsulphates, arylsulphonates, protein hydrolysates, lignosulphite waste liquors and methylcellulose. The presence of a surfactant is required if one of the active compounds and/or one of the inert carriers is insoluble in water and when the application takes place in water. The proportion of surfactants is between 5 and 40 percent by weight of the composition according to the invention.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, other additional components may also be present, for example protective colloids, binders, adhesives, thickeners, thixotropic substances, penetrants, stabilizers, sequestering agents, complex formers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% of active compound, very particularly preferably between 10 and 70% by weight.

The formulations described above can be used in a method according to the invention for controlling unwanted microorganisms, where the diaminopyrimidines according to the invention are applied to the microorganisms and/or to their habitat.

The active compounds according to the invention, as such or in their formulations, can also be used as a mixture with known fungicides, bactericides, acaricides, nematicides or insecticides, for example to widen the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is in a customary manner adapted to the use forms.

The invention furthermore includes a method for treating seed.

A further aspect of the present invention relates in particular to seed treated with at least one of the diaminopyrimidines according to the invention. The seed according to the invention is used in methods for protecting seed against animal pests and/or phytopathogenic harmful fungi. In these methods, seed treated with at least one active compound according to the invention is employed.

The active compounds or compositions according to the invention are also suitable for treating seed. A large part of the damage to crop plants caused by harmful organisms is triggered by an infection of the seed during storage or after sowing both during and after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

The control of animal pests and/or phytopathogenic fungi by treating the seed of plants has been known for a long time and is subject of continuous improvements. However, the treatment of seed entails a series of problems which cannot always be solved in a satisfactory manner. Thus, it is desirable to develop methods for protecting the seed and the germinating plant which dispense with the additional application of crop protection agents after sowing or after the emergence of the plants or where additional application is at least considerably reduced. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

Accordingly, the present invention also relates to a method for protecting seed and germinating plants against attack by animal pests and/or phytopathogenic fungi by treating the seed with a composition according to the invention. The invention also relates to the use of the compositions according to the invention for treating seed for protecting the seed and the geminating plant against phytopathogenic fungi. Furthermore, the invention relates to seed treated with a composition according to the invention for protection against phytopathogenic fungi.

The control of animal pests and/or phytopathogenic fungi which damage plants post-emergence is carried out primarily by treating the soil and the above-ground parts of plants with crop protection compositions. Owing to the concerns regarding a possible impact of the crop protection composition on the environment and the health of humans and animals, there are efforts to reduce the amount of active compounds applied.

One of the advantages of the present invention is that, because of the particular systemic properties of the compositions according to the invention, treatment of the seed with these compositions not only protects the seed itself, but also the resulting plants after emergence, from animal pests and/or phytopathogenic fungi. In this manner, the immediate treatment of the crop at the time of sowing or shortly thereafter can be dispensed with.

It is also considered to be advantageous that the active compounds or compositions according to the invention can be used in particular also for transgenic seed where the plant growing from this seed is capable of expressing a protein which acts against pests. By treating such seed with the active compounds or compositions according to the invention, even by the expression of the, for example, insecticidal protein, certain pests may be controlled. Surprisingly, a further synergistic effect may be observed here, which additionally increases the effectiveness of the protection against attack by pests.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants. The treatment of seed of cereals (such as wheat, barley, rye and oats), maize and rice is of particular importance.

As also described further below, the treatment of transgenic seed with the active compounds or compositions according to the invention is of particular importance. This refers to the seed of plants containing at least one heterologous gene which allows the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. Preferably, this heterologous gene is from Bacillus sp., the gene product having activity against the European corn borer and/or the Western corn rootworm. Particularly preferably, the heterologous gene originates from Bacillus thuringiensis.

In the context of the present invention, the composition according to the invention is applied on its own or in a suitable formulation to the seed. Preferably, the seed is treated in a stable state, so that the treatment does not cause any damage. In general, treatment of the seed may take place at any point in time between harvesting and sowing. Usually, the seed used is separated from the plant and freed from cobs, shells, stalks, coats, hairs or the flesh of the fruits. Thus, it is possible to use, for example, seed which has been harvested, cleaned and dried to a moisture content of less than 15% by weight. Alternatively, it is also possible to use seed which, after drying, has been treated, for example, with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the compositions to the seed in the form of a suitable formulation. Suitable formulations and methods for the treatment of seed are known to the person skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compounds which can be used according to the invention can be converted into customary seed dressing formulations, such as solutions, emulsions, suspensions, powders, foams, slurries or other coating materials for seed, and also ULV formulations.

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and water as well.

Suitable colorants that may be present in the seed dressing formulations to be used according to the invention include all colorants customary for such purposes. Use may be made both of pigments, of sparing solubility in water, and of dyes, which are soluble in water. Examples that may be mentioned include the colorants known under the designations Rhodamin B, C.I. Pigment Red 112 and C.I. Solvent Red 1.

Suitable wetting agents that may be present in the seed dressing formulations to be used according to the invention include all substances which promote wetting and are customary in the formulation of agrochemically active compounds. Preference is given to using alkylnaphthalenesulphonates, such as diisopropyl- or diisobutylnaphthalenesulphonates.

Suitable dispersants and/or emulsifiers that may be present in the seed dressing formulations to be used according to the invention include all nonionic, anionic and cationic dispersants which are customary in the formulation of agrochemically active compounds. Preference is given to using nonionic or anionic dispersants or mixtures of nonionic or anionic dispersants. Particularly suitable nonionic dispersants are ethylene oxide/propylene oxide block polymers, alkylphenol polyglycol ethers, and also tristryrylphenol polyglycol ethers and their phosphated or sulphated derivatives. Particularly suitable anionic dispersants are lignosulphonates, polyacrylic acid salts and arylsulphonate/formaldehyde condensates.

Defoamers that may be present in the seed dressing formulations to be used according to the invention include all foam-inhibiting compounds which are customary in the formulation of agrochemically active compounds. Preference is given to using silicone defoamers and magnesium stearate.

Preservatives that may be present in the seed dressing formulations to be used according to the invention include all compounds which can be used for such purposes in agrochemical compositions. By way of example, mention may be made of dichlorophen and benzyl alcohol hemiformal.

Secondary thickeners that may be present in the seed dressing formulations to be used according to the invention include all compounds which can be used for such purposes in agrochemical compositions. Preference is given to cellulose derivatives, acrylic acid derivatives, xanthan, modified clays and finely divided silicic acids.

Suitable adhesives that may be present in the seed dressing formulations to be used according to the invention include all customary binders which can be used in seed dressings. Polyvinylpyrrolidone, polyvinyl acetate, polyvinyl alcohol and tylose may be mentioned as being preferred.

Suitable gibberellins that may be present in the seed dressing formulations to be used according to the invention are preferably the gibberellins A1, A3 (=gibberellic acid), A4 and A7; particular preference is given to using gibberellic acid. The gibberellins are known (cf. R. Wegler “Chemie der Pflanzenschutz- and Schädlingsbekämpfungsmittel” [Chemistry of Crop Protection Agents and Pesticides], Vol. 2, Springer Verlag, 1970, pp. 401-412).

The seed dressing formulations which can be used according to the invention may be used directly or after dilution with water beforehand to treat seed of any of a very wide variety of types. For instance, the concentrates or the preparations obtainable therefrom by dilution with water may be used to dress the seed of cereals, such as wheat, barley, rye, oats, and triticale, and also the seed of maize, rice, oilseed rape, peas, beans, cotton, sunflowers, and beets, or else vegetable seed of any of a very wide variety of kinds. The seed dressing formulations which can be used according to the invention or their dilute preparations may also be used to dress seed of transgenic plants. In this context, additional synergistic effects may also arise in interaction with the substances formed by expression.

Suitable mixing equipment for treating seed with the seed dressing formulations which can be used according to the invention or the preparations prepared from them by adding water includes all mixing equipment which can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

The application rate of the seed dressing formulations which can be used according to the invention may be varied within a relatively wide range. It depends on the respective content of the active compounds in the formulations and on the seed. In general, the application rates of active compound combination are between 0.001 and 50 g per kilogram of seed, preferably between 0.01 and 15 g per kilogram of seed.

In addition, the compounds of the formulae (Ia), (Ib) and (Ic) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

Accordingly, the active compounds of the formulae (Ia), (Ib) and (Ic) according to the invention can be used both in medical and in non-medical applications.

The active compounds can be applied as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting, etc. It is furthermore possible to apply the active compounds by the ultra-low volume method or to inject the active compound preparation or the active compound itself into the soil. It is also possible to treat the seed of the plants.

When using the active compounds according to the invention as fungicides, the application rates can be varied within a relatively wide range depending on the type of application. The application rate of the active compounds according to the invention is
when treating plant parts, for example leaves: from 0,1 to 10 000 g/ha, preferably from 10 to 1 000 g/ha, particularly preferably from 50 to 300 g/ha (when the application is carried out by watering or dripping, it is even possible to reduce the application rate, especially when inert substrates such as rock wool or perlite are used);
when treating seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
when treating the soil: from 0.1 to 10 000 g/ha, preferably from 1 to 5000 g/ha.

These application rates are mentioned only in an exemplary manner and are not limiting in the sense of the invention.

In addition, the compounds according to the invention can be used for controlling a wide variety of pests, including, for example, harmful sucking insects, biting insects and other plant-parasitic pests, stored grain pests, pests which destroy technical materials, and hygienic pests as well as pests, including parasites, in the veterinary field and can be applied for their control, like for example eradication and extermination. Therefore, the present invention also encompasses a method for controlling harmful pests.

In the animal health field, i.e. in the field of veterinary medicine, the active compounds according to the present invention are active against animal parasites, in particular ectoparasites or endoparasites. The term endoparasites includes in particular helminths, such as cestodes, nematodes or trematodes, and protozoae, such as coccidia. Ectoparasites are typically and preferably arthropods, in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like.

These Parasites Include:

from the order of the Anoplurida, for example, Haematopinus spp., Linognathus spp., Pediculus spp., Phtirus spp., Solenopotes spp.; specific examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;
from the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.; specific examples are: Bovicola bovis, Bovicola ovis, Bovicola limbata, Damalina bovis, Trichodectes canis, Felicola subrostratus, Bovicola caprae, Lepikentron ovis, Werneckiella equi;
from the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; specific examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypodei bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricornis, Gasterophilus pecorum, Braula coeca;
from the order of the Siphonapterida, for example, Pulex spp., Ctenocephalides spp., Tunga spp., Xenopsylla spp., Ceratophyllus spp.; specific examples are: Ctenocephalides canis, Ctenocephalides felis, Pulex irritans, Tunga penetrans, Xenopsylla cheopis;
from the order of the Heteropterida, for example, Cimex spp., Triatoma spp., Rhodnius spp., Panstrongylus spp.
from the order of the Blattarida, for example, Blatta orientalis, Periplaneta americana, Blattela germanica, Supella spp. (for example Suppella longipalpa);
from the sub-class of the Acari (Acarina) and the orders of the Meta- and Mesostigmata, for example, Argas spp., Ornithodorus spp., Otobius spp., Ixodes spp., Amblyomma spp., Rhipicephalus (Boophilus) spp., Dermacentor spp., Haemophysalis spp., Hyalomma spp., Dermanyssus spp., Rhipicephalus spp. (the original genus of the multi-host ticks), Ornithonyssus spp., Pneumonyssus spp., Raillietia spp., Pneumonyssus spp., Sternostoma spp., Varroa spp., Acarapis spp.; specific examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalisotophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni;
from the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; specific examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neoschöngastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds according to the invention are also suitable for controlling arthropods, helminths and protozoae, which attack animals. Animals include agricultural livestock such as, for example, cattle, sheep, goats, horses, pigs, donkeys, camels, buffaloes, rabbits, chickens, turkeys, ducks, geese, cultured fish, honeybees. Moreover, animals include domestic animals—also referred to as companion animals-such as, for example, dogs, cats, cage birds, aquarium fish and what are known as experimental animals such as, for example, hamsters, guinea pigs, rats and mice.

By controlling these arthropods, helminths and/or protozoae, it is intended to reduce deaths and improve performance (in the case of meat, milk, wool, hides, eggs, honey and the like) and health of the host animal, so that more economical and simpler animal keeping is made possible by the use of the active compounds according to the invention.

For example, it is desirable to prevent or interrupt the uptake of blood by the parasites from the hosts (when applicable). Also, controlling the parasites may help to prevent the transmittance of infectious agents.

The term “controlling” as used herein with regard to the animal health field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

Generally, when used for the treatment of animals the active compounds according to the invention can be applied directly. Preferably they are applied as pharmaceutical compositions which may contain pharmaceutically acceptable excipients and/or auxiliaries which are known in the art.

In the animal health field and in animal keeping, the active compounds are applied (=administered) in the known manner by enteral administration in the form of, for example, tablets, capsules, drinks, drenches, granules, pastes, boluses, the feed-through method, suppositories; by parenteral administration, such as, for example, by injections (intramuscular, subcutaneous, intravenous, intraperitoneal and the like), implants, by nasal application, by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like. The active compounds may be formulated as shampoo or as suitable formulations usable in aerosols, unpressurized sprays, for example pump sprays and atomizer sprays.

When used for livestock, poultry, domestic animals and the like, the active compounds according to the invention can be applied as formulations (for example powders, wettable powders [“WP”], emulsions, emulsifiable concentrates [“EC”], flowables, homogeneous solutions, and suspension concentrates [“SC”]) which comprise the active compounds in an amount of from 1 to 80% by weight, either directly or after dilution (e.g. 100- to 10 000-fold dilution), or else as a chemical bath.

When used in the animal health field the active compounds according to the invention may be used in combination with suitable synergists or other active compounds, such as for example, acaricides, insecticides, anthelmintics, anti-protozoal drugs.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;
Hymenopterons, such as Sirex juvencus, Urocerus gigas, Urocerus gigas taignus, Urocerus augur;
Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, in particular hulls, screens, nets, buildings, moorings and signalling systems, against fouling.

Furthermore, the compounds according to the invention, alone or in combinations with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.
From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.
From the order of the Araneae, for example, Aviculariidae, Araneidae.
From the order of the Opiliones, for example, Pseudoscorpiones chelifer, Pseudoscorpiones cheiridium, Opiliones phalangium.
From the order of the Isopoda, for example, Oniscus asellus, Porcellio scaber.
From the order of the Diplopoda, for example, Blaniulus guttulatus, Polydesmus spp.
From the order of the Chilopoda, for example, Geophilus spp.
From the order of the Zygentoma, for example, Ctenolepisma spp., Lepisma saccharina, Lepismodes inquilinus.
From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.
From the order of the Saltatoria, for example, Acheta domesticus.
From the order of the Dermaptera, for example, Forficula auricularia.
From the order of the Isoptera, for example, Kalotermes spp., Reticulitermes spp.
From the order of the Psocoptera, for example, Lepinatus spp., Liposcelis spp.
From the order of the Coloptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.
From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.
From the order of the Lepidoptera, for example, Achroia grisella, Galleria mellonella, Plodia interpunctella, Tinea cloacella, Tinea pellionella, Tineola bisselliella.
From the order of the Siphonaptera, for example, Ctenocephalides canis, Ctenocephalides felis, Pulex irritans, Tunga penetrans, Xenopsylla cheopis.
From the order of the Hymenoptera, for example, Camponotus herculeanus, Lasius fuliginosus, Lasius niger, Lasius umbratus, Monomorium pharaonis, Paravespula spp., Tetramorium caespitum.
From the order of the Anoplura, for example, Pediculus humanus capitis, Pediculus humanus corporis, Phemphigus spp., Phylloera vastatrix, Phthirus pubis.
From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The method of treatment according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

At certain application rates, the active compound combinations according to the invention may also have a strengthening effect in plants. Accordingly, they are also suitable for mobilizing the defense system of the plant against attack by unwanted phytopathogenic fungi and/or microorganisms and/or viruses. This may, if appropriate, be one of the reasons for the enhanced activity of the combinations according to the invention, for example against fungi. Plant-strengthening (resistance-inducing) substances are to be understood as meaning, in the present context, those substances or combinations of substances which are capable of stimulating the defence system of plants in such a way that, when subsequently inoculated with unwanted phytopathogenic fungi and/or microorganisms and/or viruses, the treated plants display a substantial degree of resistance to these unwanted phytopathogenic fungi and/or microorganisms and/or viruses. In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

The plants and plant cultivars which are preferably treated according to the invention include all plants which have genes which confer particularly advantageous useful features on these plants (whether by breeding and/or biotechnological means).

Plants and plant cultivars which are also preferably treated according to the invention are resistant against one or more biotic stresses, i.e. said plants have a better defense against animal and microbial pests, such as against nematodes, insects, mites, phytopathogenic fungi, bacteria, viruses and/or viroids.

Plants and plant cultivar which can also be treated according to the invention are those plants which are resistant to one or more abiotic stress factors. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry1Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in corn events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/Home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARC encoding genes of the plants or plants cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-3-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content.
c) plants, such as oilseed rape plants, which produce oil having a low level of saturated fatty acids.

Particularly useful transgenic plants which may be treated according to the invention are plants which comprise one or more genes which encode one or more toxins and which are sold under the following trade names: YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea), for example maize. Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula (I) or the active compound mixtures according to the invention. The preferred ranges indicated above for the active compounds and mixtures also apply to the treatment of these plants. Particular emphasis is given to treating the plants with the compounds and mixtures specifically indicated in the present text.

The active compounds or compositions according to the invention can thus be employed for protecting plants for a certain period of time after treatment against attack by the pathogens mentioned. The period for which protection is provided extends generally for 1 to 28 days, preferably 1 to 14 days, particularly preferably for 1 to 10 days, very particularly preferably for 1 to 7 days after the treatment of the plants with the active compounds, or up to 200 days after a seed treatment.

The preparation and the use of the active compounds according to the invention of the formulae (Ia), (Ib) and (ft) is illustrated by the examples below. However, the invention is not limited to these examples.

### Preparation of Starting Materials of the Formula (V)

### 2,5-Dichloro-N-cyclobutylpyrimidine-4-amine (V-1)

At −10° C., 3.39 g (24.5 mmol) of potassium carbonate are added to a solution of 3.00 g (16.4 mmol) of 2,4,5-trichloropyrimidine in 50 ml of acetonitrile. 1.22 g (17.2 mmol) of cyclobutylamine are then added dropwise as a 20% strength solution in acetonitrile. With stirring, the reaction mixture is allowed to warm to room temperature overnight. The reaction mixture is stirred into 250 ml of ice-water/dilute hydrochloric acid (1:1). The mixture is extracted with ethyl acetate (2×200 ml), the combined organic phases are then washed with water (2×100 ml) and dried over MgSO₄ and the solvent is removed under reduced pressure. This gives 3.45 g (94%) of 2,5-dichloro-N-cyclobutylpyrimidine-4-amine (V-1) (logP (pH2.3): 2.62).

The following compounds can be prepared analogously:

5-Bromo-2-chloro-N-cyclobutylpyrimidine-4-amine (V-2) (logP (pH2.3): 2.87).
2-Chloro-N-cyclobutyl-5-iodopyrimidine-4-amine (V-3) (logP (pH2.3): 3.08).
2-Chloro-N-cyclobutyl-5-trifluoromethylpyrimidine-4-amine (V-4)

A mixture of 8.07 g (37.2 mmol) of 2,4-dichloro-5-trifluoropyrimidine and 12.8 g (92.9 mmol) of potassium carbonate in 150 ml acetonitrile is warmed to 50° C. 4.00 g (37.2 mmol) of cyclobutylamine hydrochloride are then added, and the mixture is stirred for 2 h. After cooling, the reaction mixture is stirred into 500 ml of ice-water and extracted with ethyl acetate (3×200 ml). The combined organic phases are separated, washed with water (2×250 ml), dried over MgSO₄ and freed from the solvent under reduced pressure. The crude product is purified by column chromatography on silica gel (cyclohexane/ethyl acetate). This gives 4.00 g (41%) of 2-chloro-N-cyclobutyl-5-trifluoromethylpyrimidine-4-amine (V-4). logP (pH2.3): 3.20

### Preparation of Compounds of the Formula (Ia)

### Method A

### 5-Chloro-N⁴-cyclobutyl-N²-(4-isopropoxyphenyl)pyrimidine-2,4-diamine hydrochloride (Compound 36)

At room temperature, 400 μl of 4 M HCl in dioxane are added to a solution of 196 mg (0.90 mmol) of 2,5-dichloro-N-(cyclobutyl)pyrimidine-4-amine and 299 mg (1.98 mmol) of 4-(isopropoxy)aniline in 12 ml acetonitrile, and the mixture is heated at 85° C. After 18 h, the hot reaction mixture is filtered and the filtrate is allowed to cool with stirring. The product, which precipitates from the filtrate, is filtered off and dried. This gives 103 mg (35%) of the desired product (logP (pH2.3): 2.21).

### Method B

### 3-1,5-Chloro-4-(cyclobutylamino)pyrimidin-2-yl]amino]benzenesulphonamide (Compound 23)

A mixture of 250 mg (1.15 mmol) of 2,5-dichloro-N-cyclobutylpyrimidine-4-amine, 247 mg (1.43 mmol) of 3-aminobenzenesulphonamide and 158 mg (0.92 mmol) of 4-toluenesulphonic acid in 12 ml of dioxane is stirred at 105° C. for 16 h. After cooling, the precipitate is filtered off, suspended in 10 ml of water, washed with water (2×10 ml) and dried. This gives 295 mg (73%) of the desired product (logP (pH2.3): 1.52).

### Preparation of Compounds of the Formula (Ib)

### N⁴-Cyclobutyl-N²-phenyl-5-(trifluoromethyl)pyrimidine-2,4-diamine (Compound 54)

A mixture of 250 mg (0.99 mmol) of 2-chloro-N-cyclobutyl-5-(trifluoromethyl)pyrimidine-4-amine, 116 mg (1.24 mmol) of aniline and 137 mg (0.80 mmol) of 4-toluenesulphonic acid in 12 ml of dioxane is stirred at 105° C. for 16 h. After cooling, the reaction mixture is stirred into ice-water and extracted with dichloromethane (3×50 ml). The combined organic phases are washed twice with in each case 10 ml of water, dried over MgSO₄ and freed from the solvent under reduced pressure. This gives 300 mg of the desired product (logP (pH2.3): 3.33).

### Preparation of Compounds of the Formula (Ic)

### 5-Bromo-N⁴-cyclobutyl-N²-(3-propoxyphenyl)pyrimidine-2,4-diamine (Compound 30)

A mixture of 150 mg (0.57 mmol) of 5-bromo-2-chloro-N-cyclobutylpyrimidine-4-amine, 104 mg (0.69 mmol) of 3-propoxyaniline and 84 mg (0.49 mmol) of 4-toluenesulphonic acid in 5 ml of dioxane is stirred at 105° C. for 18 h. After cooling, the reaction mixture is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 210 mg of the desired product (logP (pH2.3): 3.27).

### Preparation of Starting Materials of the Formula (Ix)

### 2-Anilino-5-chloropyrimidin-4(3H)-one (IX-1)

A solution consisting of 3.27 ml of a 1 M NaOH (aq) and 1 ml of water are added to a solution of 500 mg (2.73 mmol) of 2,4,5-trichloropyrimidine in 10 ml of dioxane. After 4 d of stirring at room temperature, the reaction mixture is concentrated under reduced pressure. The residue is taken up in 50 ml of ethyl acetate and neutralized with 1 N HCl (aq). The organic phase is separated off and then washed with 10 ml of water, dried over MgSO₄ and freed from the solvent under reduced pressure. The crude product is, together with 424 mg (4.55 mmol) of aniline and 532 mg (3.09 mmol) of 4-toluenesulphonic acid, taken up in 10 ml of dioxane and heated at 105° C. with stirring. After 18 h, the reaction mixture is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 1000 mg of 2-anilino-5-chloropyrimidin-4(3H)-one (IX-1) which is directly, without further purification, reacted further. logP (pH2.3): 1.56.

### Preparation of Starting Materials of the Formula (X)

### 4,5-Dichloro-N-phenylpyrimidine-2-amine (X-1)

A solution of 400 mg of 2-anilino-5-chloropyrimidin-4(3H)-one in 2 ml of phosphoryl chloride is heated at 95° C. for 18 h. After cooling, the reaction mixture is concentrated under reduced pressure, added to water and extracted with dichloromethane (3×20 ml). The combined organic phases are dried over MgSO₄ and freed from the solvent under reduced pressure. This gives 450 mg of 4,5-dichloro-N-phenylpyrimidine-2-amine (X-1) (logP (pH2.3): 3.52).

### Preparation of Compounds of the Formula (Ia)

### 5-Chloro-N⁴-(1-methylcyclobutyl)-N²-phenylpyrimidine-2,4-diamine (Compound 52)

At 0° C., 96 μl (0.69 mmol) of triethylamine and 69 mg (0.81 mmol) of 1-methylcyclobutylamine are added to a solution of 150 mg (0.63 mmol) of 4,5-dichloro-N-phenylpyrimidine-2-amine in 5 ml of acetonitrile, and the mixture is allowed to warm to room temperature overnight and stirred at 35° C. for a further 48 h. The reaction mixture is stirred into 100 ml of water and extracted with ethyl acetate (5×40 ml). The combined organic phases are dried over MgSO₄ and freed from the solvent under reduced pressure. The crude product is then purified by column chromatography on RP18 (water/acetonitrile). This gives 70 mg (39%) of the desired product (loge (pH2.3): 2.10).

### EXAMPLES

Analogously to the methods given above, it is possible to obtain the compounds of the formula I, (Ia), (Ib), (Ic) listed in Table 1 below.

I, (Ia), (Ib), (Ic)

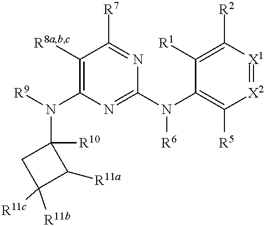

| Ex. No. | X¹ | X² | R¹ | R² or R² + | R³ R³ | R⁴ | R⁵ | R⁶ | R⁷ | R^{8a}, R^{8b}, R^{8c} | R⁹ | 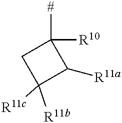 | log p |
|---|---|---|---|---|---|---|---|---|---|---|---|---|---|
| 1 | CR³ | CR⁴ | H | —CH═CH—NH— | | H | H | H | H | Br | H | cyclobutyl | 1.59[b] |
| 2 | CR³ | CR⁴ | H | —NHCH═CH— | | H | H | H | H | Br | H | cyclobutyl | 1.7[b] |
| 3 | CR³ | CR⁴ | H | —SC(SO2CH2CH3)═N— | | H | H | H | H | Br | H | cyclobutyl | 2.91[b] |
| 4 | CR³ | CR⁴ | H | —CH2CH2CONH— | | H | H | H | H | Br | H | cyclobutyl | 1.49[b] |
| 5 | CR³ | CR⁴ | H | —OCONH— | | H | H | H | H | Br | H | cyclobutyl | 1.59[b] |
| 6 | CR³ | CR⁴ | H | —NHCOCH2O— | | H | H | H | H | Br | H | cyclobutyl | 1.61[b] |
| 7 | CR³ | CR⁴ | H | —CH2CONH— | | H | H | H | H | Cl | H | cyclobutyl | 1.33[b] |
| 8 | CR³ | CR⁴ | H | —OCONH— | | H | H | H | H | Cl | H | cyclobutyl | 1.55[b] |
| 9 | CR³ | CR⁴ | H | —CH2CH2CO— | | H | H | H | H | Br | H | cyclobutyl | 2.59[b] |
| 10 | CR³ | CR⁴ | H | —N═C(CH3)S— | | H | H | H | H | Br | H | cyclobutyl | 2.11[b] |
| 11 | CR³ | CR⁴ | H | —SCOO— | | H | H | H | H | Br | H | cyclobutyl | 2.63[b] |
| 12 | CR³ | CR⁴ | H | —N(COCH3)CH2CH2— | | H | H | H | H | Br | H | cyclobutyl | 1.78[b] |
| 13 | CR³ | CR⁴ | H | —N(CH3)COCH2O— | | H | H | H | H | Br | H | cyclobutyl | 1.83[b] |
| 14 | CR³ | CR⁴ | H | —N(CH3)COCH2O— | | H | H | H | H | Br | H | cyclobutyl | 1.85[b] |
| 15 | CR³ | CR⁴ | H | —NHCOCH2CH2CH2— | | H | H | H | H | Br | H | cyclobutyl | 1.75[b] |
| 16 | CR³ | CR⁴ | H | —NHCOO— | | H | H | H | H | Br | H | cyclobutyl | 1.61[b] |
| 17 | CR³ | CR⁴ | H | —CH2CH2N(COCH3)— | | H | H | H | H | Br | H | cyclobutyl | 1.68[b] |
| 18 | CR³ | CR⁴ | H | —OCF2CF2O— | | H | H | H | H | Br | H | cyclobutyl | 4.56[b] |
| 19 | CR³ | CR⁴ | H | methylenebis(oxy) | | H | H | H | H | Br | H | cyclobutyl | 1.87[b] |
| 20 | CR³ | CR⁴ | H | —CH2SO2 CH2— | | H | H | H | H | Br | H | cyclobutyl | 1.78[b] |
| 21 | CR³ | CR⁴ | H | —N═C(CH2CH3)O— | | H | H | H | H | Br | H | cyclobutyl | 2.06[b] |
| 22 | CR³ | CR⁴ | H | —CONHCO— | | H | H | H | H | Br | H | cyclobutyl | 2.25[b] |
| 23 | CR³ | CR⁴ | H | aminosul- | H | H | H | H | H | Cl | H | cyclobutyl | 1.52[a] |
| | | | | phonyl | | | | | | | | | |
| 24 | CR³ | CR⁴ | H | CF₃ | H | SO₂NH₂ | H | H | H | Cl | H | cyclobutyl | 2.7[a] |
| 25 | CR³ | CR⁴ | H | H | (methyl- | H | H | H | H | Cl | H | cyclobutyl | 2.01[a] |
| | | | | | amino)- | | | | | | | | |
| | | | | | sulphonyl | | | | | | | | |
| 26 | CR³ | CR⁴ | H | H | (butylamino) | H | H | H | H | Cl | H | cyclobutyl | 2.91[a] |
| | | | | | sulphonyl | | | | | | | | |
| 27 | CR³ | CR⁴ | H | H | aminosul- | H | H | H | H | Cl | H | cyclobutyl | 1.62[a] |
| | | | | | phonyl | | | | | | | | |
| 28 | CR³ | CR⁴ | H | H | 4-(tert-butoxy- | H | H | H | H | Cl | H | cyclobutyl | 2.73[a] |
| | | | | | carbonyl)pip- | | | | | | | | |
| | | | | | erazin-1-yl | | | | | | | | |
| 29 | CR³ | CR⁴ | H | H | H | H | H | H | H | CN | H | cyclobutyl | 2.92[b] |
| 30 | CR³ | CR⁴ | H | propoxy | H | H | H | H | H | Br | H | cyclobutyl | 3.27[b] |
| 31 | CR³ | CR⁴ | H | methoxy | F | H | H | H | H | Br | H | cyclobutyl | 2.41[b] |
| 32 | CR³ | CR⁴ | H | acetyl | H | H | H | H | H | Br | H | cyclobutyl | 2.28[b] |
| 33 | CR³ | CR⁴ | H | 1,3-thiazol- | H | H | H | H | H | Br | H | cyclobutyl | 2.8[b] |
| | | | | 2-yl | | | | | | | | | |
| 34 | CR³ | CR⁴ | H | H | 2,5-dioxo- | H | H | H | H | Br | H | cyclobutyl | 1.62[b] |
| | | | | | pyrrolidin-1-yl | | | | | | | | |
| 35 | CR³ | CR⁴ | H | H | ethoxy | H | H | H | H | Br | H | cyclobutyl | 2.17[b] |
| 36 | CR³ | CR⁴ | H | H | isopropoxy | H | H | H | H | Br | H | cyclobutyl | 2.44[b] |
| 37 | CR³ | CR⁴ | H | H | OH | H | H | H | H | Br | H | cyclobutyl | 1.27[b] |
| 38 | CR³ | CR⁴ | H | methoxy | methoxy | MeO | H | H | H | Br | H | cyclobutyl | 2.04[b] |
| 39 | CR³ | CR⁴ | H | methoxy- | H | H | H | H | H | Br | H | cyclobutyl | 2.2[b] |
| | | | | methyl | | | | | | | | | |
| 40 | CR³ | CR⁴ | H | H | isopropyl | H | H | H | H | Br | H | cyclobutyl | 3.13[b] |
| 41 | CR³ | CR⁴ | H | H | 2-methoxy-1- | H | H | H | H | Br | H | cyclobutyl | 2.11[b] |
| | | | | | methylethoxy | | | | | | | | |
| 42 | CR³ | CR⁴ | H | methoxy- | H | H | H | H | H | Cl | H | cyclobutyl | 2.05[b] |
| | | | | methyl | | | | | | | | | |
| 43 | CR³ | CR⁴ | H | H | [(2-chloro- | H | H | H | H | Br | H | cyclobutyl | 2.06[b] |
| | | | | | ethoxy)car- | | | | | | | | |
| | | | | | bonyl]amino | | | | | | | | |
| 44 | CR³ | CR⁴ | H | methylthio | H | H | H | H | H | Br | H | cyclobutyl | 2.92[b] |
| 45 | CR³ | CR⁴ | H | 2-oxopyrro- | H | H | H | H | H | Br | H | cyclobutyl | 1.84[b] |
| | | | | lidin-1-yl | | | | | | | | | |
| 46 | CR³ | CR⁴ | H | H | (methoxycar- | H | H | H | H | Br | H | cyclobutyl | 1.62[b] |
| | | | | | bonyl)-amino | | | | | | | | |
| 47 | CR³ | CR⁴ | H | H | F | H | H | H | H | Cl | H | cyclobutyl | 2.15[a]; |
| | | | | | | | | | | | | | 3.7[c] |
| 48 | CR³ | CR⁴ | H | 2-oxopyrro- | H | H | H | H | H | Cl | H | cyclobutyl | 1.71[a]; |
| | | | | lidin-1-yl | | | | | | | | | 2.74[c] |
| 49 | CR³ | CR⁴ | H | H | F | H | H | H | H | Br | H | cyclobutyl | 2.33[b] |
| 50 | CR³ | CR⁴ | H | H | 3-oxomor- | H | H | H | H | Br | H | cyclobutyl | 1.53[b] |
| | | | | | pholin4-yl | | | | | | | | |
| 51 | CR³ | CR⁴ | H | 3,5-dimethyl- | H | H | H | H | H | Cl | H | cyclobutyl | 2.94[a]; |
| | | | | piperidin-1-yl | | | | | | | | | 5.82[c] |
| 52 | CR³ | CR⁴ | H | H | H | H | H | H | H | Cl | H | 1-methyl- | 2.1[b] |
| | | | | | | | | | | | | cyclobutyl | |
| 53 | CR³ | CR⁴ | H | methoxy | H | OMe | H | H | H | Br | H | cyclobutyl | 2.12[b] |
| 54 | CR³ | CR⁴ | H | H | H | H | H | H | H | CF₃ | H | cyclobutyl | 3.33[a]; |
| | | | | | | | | | | | | | 3.96[c] |
| 55 | CR³ | CR⁴ | H | 2-oxopyrro- | H | H | H | H | H | CF₃ | H | cyclobutyl | 2.64[a]; |
| | | | | lidin-1-yl | | | | | | | | | 3.09[c] |
| 56 | CR³ | CR⁴ | H | H | 2-oxopyrrolid- | H | H | H | H | CF₃ | H | cyclobutyl | 2.51[a]; |
| | | | | | in-1-yl | | | | | | | | 3.01[c] |
| 57 | CR³ | CR⁴ | H | methylthio | H | H | H | H | H | CF₃ | H | cyclobutyl | 4.05[a]; |
| | | | | | | | | | | | | | 4.36[c] |
| 58 | CR³ | CR⁴ | H | H | F | H | H | H | H | CF₃ | H | cyclobutyl | 3.5[a]; |
| | | | | | | | | | | | | | 4[c] |
| 59 | CR³ | CR⁴ | H | 3-methyl-2- | H | H | H | H | H | Br | H | cyclobutyl | 1.79[b] |
| | | | | oxo-imidazo- | | | | | | | | | |
| | | | | lidin-1-yl | | | | | | | | | |
| 60 | CR³ | CR⁴ | Me | H | H | H | H | H | H | Br | H | cyclobutyl | 1.87[b] |
| 61 | CR³ | CR⁴ | H | Me | H | Me | Me | H | H | Br | H | cyclobutyl | 2.57[b] |
| 62 | CR³ | CR⁴ | H | H | F | H | H | H | H | Br | H | cyclobutyl | 2.6[b] |
| 63 | CR³ | CR⁴ | H | H | CF₃ | H | F | H | H | Br | H | cyclobutyl | 3.9[b] |
| 64 | CR³ | CR⁴ | H | cyano | H | H | H | H | H | Br | H | cyclobutyl | 2.67[b] |
| 65 | CR³ | CR⁴ | H | OMe | OMe | H | H | H | H | Br | H | cyclobutyl | 1.71[b] |
| 66 | CR³ | CR⁴ | H | [(4-methyl- | Me | H | H | H | H | Br | H | cyclobutyl | 3.07[b] |
| | | | | phenyl)amino] | | | | | | | | | |
| | | | | sulphonyl | | | | | | | | | |
| 67 | CR³ | CR⁴ | H | H | Br | H | H | H | H | Br | H | cyclobutyl | 3.18[b] |
| 68 | CR³ | CR⁴ | H | H | H | H | O- | H | H | Br | H | cyclobutyl | 2.23[b] |
| | | | | | | | Me | | | | | | |
| 69 | CR³ | CR⁴ | H | H | Cl | H | H | H | H | Br | H | cyclobutyl | 2.99[b] |
| 70 | CR³ | CR⁴ | H | H | CN | H | H | H | H | Br | H | cyclobutyl | 2.95[b] |
| 71 | CR³ | CR⁴ | H | H | (trifluoro- | H | H | H | H | Br | H | cyclobutyl | 4.51[b] |
| | | | | | methyl)thio | | | | | | | | |
| 72 | CR³ | CR⁴ | H | H | anilinocar- | H | H | H | H | Br | H | cyclobutyl | 2.09[b] |
| | | | | | bonyl | | | | | | | | |
| 73 | CR³ | CR⁴ | H | Cl | methylthio | H | H | H | H | Br | H | cyclobutyl | 3.5[b] |
| 74 | CR³ | CR⁴ | H | H | 1,3-thiazol- | H | H | H | H | Br | H | cyclobutyl | 2.26[b] |
| | | | | | 4-yl | | | | | | | | |
| 75 | CR³ | CR⁴ | H | H | difluoro- | H | H | H | H | Br | H | cyclobutyl | 2.5[b] |
| | | | | | methoxy | | | | | | | | |
| 76 | CR³ | CR⁴ | H | Cl | H | Cl | H | H | H | Br | H | cyclobutyl | 4.98[b] |
| 77 | CR³ | CR⁴ | H | ethoxycar- | H | H | H | H | H | Br | H | cyclobutyl | 2.77[b] |
| | | | | bonyl | | | | | | | | | |
| 78 | CR³ | CR⁴ | H | Cl | methoxy- | H | H | H | H | Br | H | cyclobutyl | 3.59[b] |
| | | | | | carbonyl | | | | | | | | |
| 79 | CR³ | CR⁴ | H | aminosul- | Cl | H | H | H | H | Br | H | cyclobutyl | 2.02[b] |
| | | | | phonyl | | | | | | | | | |
| 80 | CR³ | CR⁴ | H | H | OH | H | F | H | H | Br | H | cyclobutyl | 1.55[b] |
| 81 | CR³ | CR⁴ | H | Cl | trifluoro- | H | H | H | H | Br | H | cyclobutyl | 4.72[b] |
| | | | | | methoxy | | | | | | | | |
| 82 | CR³ | CR⁴ | H | acryloylamino | H | H | H | H | H | Br | H | cyclobutyl | 1.71[b] |
| 83 | CR³ | CR⁴ | H | H | allylcarbamoyl | H | H | H | H | Br | H | cyclobutyl | 1.99[b] |
| 84 | CR³ | CR⁴ | H | OMe | (methylsul- | H | H | H | H | Br | H | cyclobutyl | 1.75[b] |
| | | | | | phonyl)amino | | | | | | | | |
| 85 | CR³ | CR⁴ | H | H | (methylsul- | H | H | H | H | Br | H | cyclobutyl | 1.61[b] |
| | | | | | phonyl)amino | | | | | | | | |
| 86 | CR³ | CR⁴ | H | H | [(methyl- | H | H | H | H | Br | H | cyclobutyl | 1.71[b] |
| | | | | | amino)sul- | | | | | | | | |
| | | | | | phonyl]methyl | | | | | | | | |
| 87 | CR³ | CR⁴ | H | (methylsul- | H | H | H | H | H | Br | H | cyclobutyl | 1.73[b] |
| | | | | phonyl)amino | | | | | | | | | |
| 88 | CR³ | CR⁴ | H | (methylsul- | H | H | H | H | H | Br | H | cyclobutyl | 1.66[b] |
| | | | | phonyl)methyl | | | | | | | | | |
| 89 | CR³ | CR⁴ | H | [(dimethyl- | H | H | H | H | H | Br | H | cyclobutyl | 2.7[b] |
| | | | | amino)sul- | | | | | | | | | |
| | | | | phonyl]oxy | | | | | | | | | |
| 90 | CR³ | CR⁴ | H | OMe | acetamido | H | H | H | H | Br | H | cyclobutyl | 1.59[b] |
| 91 | CR³ | CR⁴ | H | H | (dimethyl- | H | H | H | H | Br | H | cyclobutyl | 2.73[b] |
| | | | | | amino)sul- | | | | | | | | |
| | | | | | phonyl | | | | | | | | |
| 92 | CR³ | CR⁴ | H | H | (2,2- | H | H | H | H | Br | H | cyclobutyl | 2.02[b] |
| | | | | | dimethylpro- | | | | | | | | |
| | | | | | panoyl) | | | | | | | | |
| | | | | | amino | | | | | | | | |
| 93 | CR³ | CR⁴ | H | H | acetyl(ethyl) | H | H | H | H | Br | H | cyclobutyl | 1.92[b] |
| | | | | | amino | | | | | | | | |
| 94 | CR³ | CR⁴ | H | H | acetyl(methyl) | H | H | H | H | Br | H | cyclobutyl | 1.73[b] |
| | | | | | amino | | | | | | | | |
| 95 | CR³ | CR⁴ | H | H | (trifluoro- | H | H | H | H | Br | H | cyclobutyl | 2.21[b] |
| | | | | | acetyl)amino | | | | | | | | |
| 96 | CR³ | CR⁴ | H | acetamido | H | H | H | H | H | Br | H | cyclobutyl | 1.54[b] |
| 97 | CR³ | CR⁴ | H | propionyl- | H | H | H | H | H | Br | H | cyclobutyl | 1.71[b] |
| | | | | amino | | | | | | | | | |
| 98 | CR³ | CR⁴ | H | methacryloyl- | H | H | H | H | H | Br | H | cyclobutyl | 1.9[b] |
| | | | | amino | | | | | | | | | |
| 99 | CR³ | CR⁴ | H | (2,2-di- | H | H | H | H | H | Br | H | cyclobutyl | 2.11[b] |
| | | | | methylpro- | | | | | | | | | |
| | | | | panoyl)amino | | | | | | | | | |
| 100 | CR³ | CR⁴ | H | H | dimethylcar- | H | H | H | H | Br | H | cyclobutyl | 1.73[b] |
| | | | | | bamoyl | | | | | | | | |
| 101 | CR³ | CR⁴ | H | H | methylcar- | H | H | H | H | Br | H | cyclobutyl | 1.54[b] |
| | | | | | bamoyl | | | | | | | | |
| 102 | CR³ | CR⁴ | H | H | tert-butylcar- | H | H | H | H | Br | H | cyclobutyl | 2.46[b] |
| | | | | | bamoyl | | | | | | | | |
| 103 | CR³ | CR⁴ | H | piperidin-1- | H | H | H | H | H | Br | H | cyclobutyl | |
| | | | | yl-sulphonyl | | | | | | | | | |
| 104 | CR³ | CR⁴ | H | H | 3,3-dimethyl- | H | H | H | H | Br | H | cyclobutyl | 2.63[b] |
| | | | | | 2-oxobutyl | | | | | | | | |
| 105 | CR³ | CR⁴ | H | (ethoxycar- | H | H | H | H | H | Br | H | cyclobutyl | 2.04[b] |
| | | | | bonyl)amino | | | | | | | | | |
| 106 | CR³ | CR⁴ | H | (methoxycar- | H | H | H | H | H | Br | H | cyclobutyl | 1.83[b] |
| | | | | bonyl)amino | | | | | | | | | |
| 107 | CR³ | CR⁴ | H | CN | H | H | H | H | H | Cl | H | cyclobutyl | 2.46[b] |
| 108 | CR³ | CR⁴ | H | OMe | acetamido | H | H | H | H | Cl | H | cyclobutyl | 1.55[b] |
| 109 | CR³ | CR⁴ | H | OMe | H | H | H | H | H | Cl | H | cyclobutyl | 2.06[b] |
| 110 | CR³ | CR⁴ | H | H | OMe | H | H | H | H | Cl | H | cyclobutyl | 1.75[b] |
| 111 | CR³ | CR⁴ | H | H | CN | H | H | H | H | Cl | H | cyclobutyl | 2.77[b] |
| 112 | CR³ | CR⁴ | H | trifluoro- | H | H | H | H | H | Cl | H | cyclobutyl | 3.59[b] |
| | | | | methoxy | | | | | | | | | |
| 113 | CR³ | CR⁴ | H | H | methylsul- | H | H | H | H | Cl | H | cyclobutyl | 2.09[b] |
| | | | | | phonyl | | | | | | | | |
| 114 | CR³ | CR⁴ | H | H | (dimethyl- | H | H | H | H | Cl | H | cyclobutyl | 2.53[b] |
| | | | | | amino)sul- | | | | | | | | |
| | | | | | phonyl | | | | | | | | |
| 115 | CR³ | CR⁴ | H | Cl | methylthio | H | H | H | H | Cl | H | cyclobutyl | 3.22[b] |
| 116 | CR³ | CR⁴ | H | H | (2,2- | H | H | H | H | Cl | H | cyclobutyl | 1.94[b] |
| | | | | | dimethylpro- | | | | | | | | |
| | | | | | panoyl)-amino | | | | | | | | |
| 117 | CR³ | CR⁴ | H | H | acetamido | H | H | H | H | Cl | H | cyclobutyl | 1.43[b] |
| 118 | CR³ | CR⁴ | H | Cl | acetamido | H | H | H | H | Cl | H | cyclobutyl | 1.78[b] |
| 119 | CR³ | CR⁴ | H | H | acetyl(ethyl) | H | H | H | H | Cl | H | cyclobutyl | 1.83[b] |
| | | | | | amino | | | | | | | | |
| 120 | CR³ | CR⁴ | H | H | acetyl(methyl) | H | H | H | H | Cl | H | cyclobutyl | 1.64[b] |
| | | | | | amino | | | | | | | | |
| 121 | CR³ | CR⁴ | H | H | (trifluoroacetyl) | H | H | H | H | Cl | H | cyclobutyl | 2.09[b] |
| | | | | | amino | | | | | | | | |
| 122 | CR³ | CR⁴ | H | H | [(1-methyl- | H | H | H | H | Cl | H | cyclobutyl | 1.87[b] |
| | | | | | cyclopropyl) | | | | | | | | |
| | | | | | carbonyl] | | | | | | | | |
| | | | | | amino | | | | | | | | |
| 123 | CR³ | CR⁴ | H | acetamido | H | H | H | H | H | Cl | H | cyclobutyl | 1.51[b] |
| 124 | CR³ | CR⁴ | H | methylacryl- | H | H | H | H | H | Cl | H | cyclobutyl | 1.8[b] |
| | | | | oylamino | | | | | | | | | |
| 125 | CR³ | CR⁴ | H | (2,2-di- | H | H | H | H | H | Cl | H | cyclobutyl | 2.04[b] |
| | | | | methyl)pro- | | | | | | | | | |
| | | | | panoyl)amino | | | | | | | | | |
| 126 | CR³ | CR⁴ | H | H | methylcar- | H | H | H | H | Cl | H | cyclobutyl | 1.5[b] |
| | | | | | bamoyl | | | | | | | | |
| 127 | CR³ | CR⁴ | H | methylcar- | H | H | H | H | H | Cl | H | cyclobutyl | 1.5[b] |
| | | | | bamoyl | | | | | | | | | |
| 128 | CR³ | CR⁴ | H | methylthio | H | H | H | H | H | Cl | H | cyclobutyl | 2.46[b] |
| 129 | CR³ | CR⁴ | H | H | methylthio | H | H | H | H | Cl | H | cyclobutyl | 2.33[b] |
| 130 | CR³ | CR⁴ | H | piperidin-1- | H | H | H | H | H | Cl | H | cyclobutyl | 2.3[b] |
| | | | | ylsulphonyl | | | | | | | | | |
| 131 | CR³ | CR⁴ | H | H | acetyl | H | H | H | H | Cl | H | cyclobutyl | 2.26[b] |
| 132 | CR³ | CR⁴ | H | H | 2-oxo-1,3-oxa- | H | H | H | H | Cl | H | cyclobutyl | 1.59[b] |
| | | | | | zolidin-3-yl | | | | | | | | |
| 133 | CR³ | CR⁴ | H | H | 2-oxopyrro- | H | H | H | H | Cl | H | cyclobutyl | 1.66[b] |
| | | | | | lidin-1-yl | | | | | | | | |
| 134 | CR³ | CR⁴ | H | H | 2,5-dioxoimi- | H | H | H | H | Cl | H | cyclobutyl | 1.33[b] |
| | | | | | dazolidin-4-yl | | | | | | | | |
| 135 | CR³ | CR⁴ | H | H | (methoxycar- | H | H | H | H | Cl | H | cyclobutyl | 1.61[b] |
| | | | | | bonyl)amino | | | | | | | | |
| 136 | CR³ | CR⁴ | H | H | (ethoxy- | H | H | H | H | Cl | H | cyclobutyl | 1.8[b] |
| | | | | | carbonyl) | | | | | | | | |
| | | | | | amino | | | | | | | | |
| 137 | CR³ | CR⁴ | H | (ethoxycar- | H | H | H | H | H | Cl | H | cyclobutyl | 1.94[b] |
| | | | | bonyl)amino | | | | | | | | | |
| 138 | CR³ | CR⁴ | H | (methylcar- | H | H | H | H | H | Cl | H | cyclobutyl | 1.64[b] |
| | | | | bamoyl)oxy | | | | | | | | | |
| 139 | CR³ | CR⁴ | H | (methoxycar- | H | H | H | H | H | Cl | H | cyclobutyl | 1.73 |
| | | | | bonyl)amino | | | | | | | | | |
| 140 | CR³ | CR⁴ | H | [(1-methyl- | H | H | H | H | H | Cl | H | cyclobutyl | 1.92[b] |
| | | | | cyclopropyl) | | | | | | | | | |
| | | | | carbonyl]amino | | | | | | | | | |
| 141 | CR³ | CR⁴ | H | (3-fluoro-2,2- | H | H | H | H | H | Cl | H | cyclobutyl | 1.97[b] |
| | | | | dimethylpro- | | | | | | | | | |
| | | | | panoyl)amino | | | | | | | | | |
| 142 | CR³ | CR⁴ | H | acetamido | Me | H | H | H | H | Cl | H | cyclobutyl | 1.38[b] |
| 143 | CR³ | CR⁴ | H | 2-(tert-butyl- | H | H | H | H | H | Br | H | cyclobutyl | 2.19[b] |
| | | | | amino)2-oxo- | | | | | | | | | |
| | | | | ethyl | | | | | | | | | |
| 144 | CR³ | CR⁴ | H | F | H | F | H | H | H | Br | H | cyclobutyl | 3.98[b] |
| 145 | CR³ | CR⁴ | H | H | acetyl | H | H | H | H | Br | H | cyclobutyl | 2.65[b] |
| 146 | CR³ | CR⁴ | H | isopropylthio | H | H | H | H | H | Cl | H | cyclobutyl | 3.62[b] |
| 147 | CR³ | CR⁴ | H | morpholin-4- | H | H | H | H | H | Br | H | cyclobutyl | 2.46[b] |
| | | | | ylsulphonyl | | | | | | | | | |
| 148 | CR³ | CR⁴ | H | H | 2-ethoxy-2- | H | H | H | H | Br | H | cyclobutyl | 2.3[b] |
| | | | | | oxo-ethyl | | | | | | | | |
| 149 | CR³ | CR⁴ | H | H | cyanoacetyl | H | H | H | H | Br | H | cyclobutyl | 2.57[b] |
| 150 | CR³ | CR⁴ | H | acetyl | Cl | H | H | H | H | Br | H | cyclobutyl | 2.95[b] |
| 151 | CR³ | CR⁴ | H | OH | (ethoxycar- | H | H | H | H | Br | H | cyclobutyl | 1.8[b] |
| | | | | | bonyl)amino | | | | | | | | |
| 152 | CR³ | CR⁴ | H | H | formamido | H | H | H | H | Br | H | cyclobutyl | 1.47[b] |
| 153 | CR³ | CR⁴ | H | H | formyl | H | H | H | H | Br | H | cyclobutyl | 1.68[b] |
| | | | | | (methyl)amino | | | | | | | | |
| 154 | CR³ | CR⁴ | H | formamido | H | H | H | H | H | Br | H | cyclobutyl | 1.58[b] |
| 155 | CR³ | CR⁴ | H | dimethylcar- | H | H | H | H | H | Br | H | cyclobutyl | 1.68[b] |
| | | | | bamoyl | | | | | | | | | |
| 156 | CR³ | CR⁴ | H | H | COOH | H | H | H | H | Br | H | cyclobutyl | 1.94[b] |
| 157 | CR³ | CR⁴ | H | (dimethyl- | H | H | H | H | H | Br | H | cyclobutyl | 2.53[b] |
| | | | | amino)sul- | | | | | | | | | |
| | | | | phonyl | | | | | | | | | |
| 158 | CR³ | CR⁴ | H | H | methoxy- | H | H | H | H | Br | H | cyclobutyl | 2.84[b] |
| | | | | | carbonyl | | | | | | | | |
| 159 | CR³ | CR⁴ | H | H | 1,1,2,2- | H | H | H | H | Br | H | cyclobutyl | 3.1[b] |
| | | | | | tetrafluoro- | | | | | | | | |
| | | | | | ethoxy | | | | | | | | |
| 160 | CR³ | CR⁴ | H | H | [(dimethyl- | H | H | H | H | Br | H | cyclobutyl | 2.46[b] |
| | | | | | amino)- | | | | | | | | |
| | | | | | sulphonyl]oxy | | | | | | | | |
| 161 | CR³ | CR⁴ | H | H | pentanoyloxy | H | H | H | H | Br | H | cyclobutyl | 4.32[b] |
| 162 | CR³ | CR⁴ | H | H | (tert-butoxy- | H | H | H | H | Br | H | cyclobutyl | 2.36[b] |
| | | | | | carbonyl)amino | | | | | | | | |
| 163 | CR³ | CR⁴ | H | H | acetyl(cyclo- | H | H | H | H | Br | H | cyclobutyl | 2.7[b] |
| | | | | | hexyl)amino | | | | | | | | |
| 164 | CR³ | CR⁴ | H | H | 3-methyl-2,5- | H | H | H | H | Br | H | cyclobutyl | 1.63[b] |
| | | | | | dioxo- | | | | | | | | |
| | | | | | imidazo- | | | | | | | | |
| | | | | | lidin-1-yl | | | | | | | | |
| 165 | CR³ | CR⁴ | H | H | (3-chloro-2,2- | H | H | H | H | Br | H | cyclobutyl | 2.16[b] |
| | | | | | dimethylpro- | | | | | | | | |
| | | | | | panoyl)amino | | | | | | | | |
| 166 | CR³ | CR⁴ | H | (3-fluoro-2,2- | H | H | H | H | H | Br | H | cyclobutyl | 2.09[b] |
| | | | | dimethylpro- | | | | | | | | | |
| | | | | panoyl)amino | | | | | | | | | |
| 167 | CR³ | CR⁴ | H | 2-furoylamino | H | H | H | H | H | Br | H | cyclobutyl | 1.94[b] |
| 168 | CR³ | CR⁴ | H | benzoylamino | H | H | H | H | H | Br | H | cyclobutyl | 2.46[b] |
| 169 | CR³ | CR⁴ | H | H | isobutyryl- | H | H | H | H | Br | H | cyclobutyl | 1.83[b] |
| | | | | | amino | | | | | | | | |
| 170 | CR³ | CR⁴ | H | 1,1,2,2-tetra- | H | H | H | H | H | Br | H | cyclobutyl | 3.37[b] |
| | | | | fluoroethoxy | | | | | | | | | |
| 171 | CR³ | CR⁴ | H | H | (3-fluoro-2,2- | H | H | H | H | Br | H | cyclobutyl | 1.99[b] |
| | | | | | dimethylpro- | | | | | | | | |
| | | | | | panoyl)amino | | | | | | | | |
| 172 | CR³ | CR⁴ | H | H | propionyl | H | H | H | H | Br | H | cyclobutyl | 3.19[b] |
| 173 | CR³ | CR⁴ | H | H | 2-oxo-1,3-oxa- | H | H | H | H | Br | H | cyclobutyl | 1.62[b] |
| | | | | | zolidin-3-yl | | | | | | | | |
| 174 | CR³ | CR⁴ | H | H | 2-oxopyrro- | H | H | H | H | Br | H | cyclobutyl | 1.72[b] |
| | | | | | idin-1 | | | | | | | | |
| 175 | CR³ | CR⁴ | H | methylthio | Me | H | H | H | H | Br | H | cyclobutyl | 3.08[b] |
| 176 | CR³ | CR⁴ | H | H | [(2- | H | H | H | H | Br | H | cyclobutyl | 1.13[b] |
| | | | | | methoxyethyl) | | | | | | | | |
| | | | | | amino]-methyl | | | | | | | | |
| 177 | CR³ | CR⁴ | H | acetyl | OH | H | H | H | H | Br | H | cyclobutyl | 2.09[b] |
| 178 | CR³ | CR⁴ | H | OH | H | H | H | H | H | Br | H | cyclobutyl | 1.5[b] |
| 179 | CR³ | CR⁴ | H | H | methylsul- | H | H | H | H | Br | H | cyclobutyl | 2.32[b] |
| | | | | | phonyl | | | | | | | | |
| 180 | CR³ | CR⁴ | H | cyclopropyl- | H | H | H | H | H | Br | H | cyclobutyl | 1.78[b] |
| | | | | carbamoyl | | | | | | | | | |
| 181 | CR³ | CR⁴ | H | (2,2,2-tri- | H | H | H | H | H | Br | H | cyclobutyl | 2.21[b] |
| | | | | fluoroethyl)- | | | | | | | | | |
| | | | | carbamoyl | | | | | | | | | |
| 182 | CR³ | CR⁴ | H | trifluoro- | H | H | H | H | H | Br | H | cyclobutyl | 4.15[b] |
| | | | | methoxy | | | | | | | | | |
| 183 | CR³ | CR⁴ | H | H | trifluoro- | H | H | H | H | Br | H | cyclobutyl | 3.73[b] |
| | | | | | methoxy | | | | | | | | |
| 184 | CR³ | CR⁴ | H | F | OH | H | H | H | H | Br | H | cyclobutyl | 1.5[b] |
| 185 | CR³ | CR⁴ | H | hydroxy- | H | H | H | H | H | Br | H | cyclobutyl | 1.44[b] |
| | | | | methyl | | | | | | | | | |
| 186 | CR³ | CR⁴ | H | Cl | acetamido | H | H | H | H | Br | H | cyclobutyl | 1.97[b] |
| 187 | CR³ | CR⁴ | H | H | 2,5-dioxoimida- | H | H | H | H | Br | H | cyclobutyl | 1.24[b] |
| | | | | | zolidin-4-yl | | | | | | | | |
| 188 | CR³ | CR⁴ | H | ethylthio | H | H | H | H | H | Cl | H | cyclobutyl | 3.15[b] |
| 189 | CR³ | CR⁴ | H | H | [(1- | H | H | H | H | Br | H | cyclobutyl | 2[b] |
| | | | | | methylcyclo- | | | | | | | | |
| | | | | | propyl)- | | | | | | | | |
| | | | | | carbonyl]amino | | | | | | | | |
| 190 | CR³ | CR⁴ | H | ethylcar- | H | H | H | H | H | Br | H | cyclobutyl | 1.73[b] |
| | | | | bamoyl | | | | | | | | | |
| 191 | CR³ | CR⁴ | H | H | morpholin-4- | H | H | H | H | Br | H | cyclobutyl | 1.75[b] |
| | | | | | ylcarbonyl | | | | | | | | |
| 192 | CR³ | CR⁴ | H | methylcar- | H | H | H | H | H | Br | H | cyclobutyl | 1.58[b] |
| | | | | bamoyl | | | | | | | | | |
| 193 | CR³ | CR⁴ | H | H | (trifluoro- | H | H | H | H | Br | H | cyclobutyl | 4.32[b] |
| | | | | | methyl)sul- | | | | | | | | |
| | | | | | phonyl; | | | | | | | | |
| 194 | CR³ | CR⁴ | H | propionyl | OH | H | H | H | H | Br | H | cyclobutyl | 2.39[b] |
| 195 | CR³ | CR⁴ | H | H | (ethoxycar- | H | H | H | H | Br | H | cyclobutyl | 1.9[b] |
| | | | | | bonyl)amino | | | | | | | | |
| 196 | CR³ | CR⁴ | H | (methylcar- | H | H | H | H | H | Br | H | cyclobutyl | 1.75[b] |
| | | | | bamoyl)oxy | | | | | | | | | |
| 197 | CR³ | CR⁴ | H | H | trifluoroacetyl | H | H | H | H | Br | H | cyclobutyl | 4.41[b] |
| 198 | CR³ | CR⁴ | H | H | OH | H | Me | H | H | Br | H | cyclobutyl | 1.47[b] |
| 199 | CR³ | CR⁴ | H | (phenoxycar- | H | H | H | H | H | Br | H | cyclobutyl | 2.51[b] |
| | | | | bonyl)amino | | | | | | | | | |
| 200 | CR³ | CR⁴ | H | acetamido | Me | H | H | H | H | Br | H | cyclobutyl | 1.58[b] |
| 201 | CR³ | CR⁴ | H | Me | OH | H | H | H | H | Br | H | cyclobutyl | 1.59[b] |
| 202 | CR³ | CR⁴ | H | H | (difluoro- | H | H | H | H | Br | H | cyclobutyl | 3.41[b] |
| | | | | | methyl)thio | | | | | | | | |
| 203 | CR³ | CR⁴ | H | H | piperidin-1-yl- | H | H | H | H | Cl | H | cyclobutyl | 2.27[b] |
| | | | | | carbonyl | | | | | | | | |
| 204 | CR³ | CR⁴ | H | 2-oxopiper- | H | H | H | H | H | Cl | H | cyclobutyl | 1.66[b] |
| | | | | idin-1-yl | | | | | | | | | |
| 205 | CR³ | CR⁴ | H | isopropyl | H | H | H | H | H | Br | H | cyclobutyl | 3.21[b] |
| 206 | CR³ | CR⁴ | H | 3-(2-chloro- | H | H | H | H | H | Br | H | cyclobutyl | 2.19[b] |
| | | | | ethyl)- | | | | | | | | | |
| | | | | 2-oxoimida- | | | | | | | | | |
| | | | | zolidin-1-yl | | | | | | | | | |
| 207 | CR³ | CR⁴ | H | H | cyclopropyl- | | H | H | H | Cl | H | cyclobutyl | 0.71[b] |
| | | | | | amino | | | | | | | | |
| 208 | CR³ | CR⁴ | H | H | OMe | | H | H | H | Br | H | cyclobutyl | 1.8[b] |
| 209 | CR³ | CR⁴ | H | H | acetyl(cyclo- | H | H | H | H | Br | H | cyclobutyl | 1.9[b] |
| | | | | | propyl)amino | | | | | | | | |
| 210 | CR³ | CR⁴ | H | 3-oxomor- | H | H | H | H | H | Br | H | cyclobutyl | 1.53[b] |
| | | | | pholin-4-yl | | | | | | | | | |
| 211 | CR³ | CR⁴ | H | 2-oxo- | F | H | H | H | H | CF₃ | H | cyclobutyl | 2.69[a]; |
| | | | | pyrro- | | | | | | | | | 2.96[c] |
| | | | | lidin-1-yl | | | | | | | | | |
| 212 | CR³ | CR⁴ | H | 2-oxo- | F | H | H | H | H | Cl | H | cyclobutyl | 1.75[a] |
| | | | | pyrro- | | | | | | | | | |
| | | | | lidin-1-yl | | | | | | | | | |
| 213 | CR³ | CR⁴ | H | H | methylamino | H | H | H | H | Br | H | cyclobutyl | 1.18[b] |
| 214 | CR³ | CR⁴ | H | 2,2,2-trifluoro- | H | H | H | H | H | Br | H | cyclobutyl | 2.17[b] |
| | | | | 1-hydroxy- | | | | | | | | | |
| | | | | ethyl | | | | | | | | | |
| 215 | CR³ | CR⁴ | H | 2-oxoazepan- | H | H | H | H | H | Br | H | cyclobutyl | 2.08[a] |
| | | | | 1-yl | | | | | | | | | |
| 216 | CR³ | CR⁴ | H | 2-oxoazepan- | H | H | H | H | H | Cl | H | cyclobutyl | 1.96[a] |
| | | | | 1-yl | | | | | | | | | |
| 217 | CR³ | CR⁴ | H | 2-oxopiper- | H | H | H | H | H | Br | H | cyclobutyl | 1.77[a] |
| | | | | idin-1-yl | | | | | | | | | |
| 218 | CR³ | CR⁴ | H | 2,2,2-trifluoro- | H | H | H | H | H | Cl | H | cyclobutyl | 2.04[b] |
| | | | | 1-hydroxy- | | | | | | | | | |
| | | | | ethyl | | | | | | | | | |
| 219 | CR³ | CR⁴ | H | cyanomethyl | F | H | H | H | H | Br | H | cyclobutyl | 2.2[b] |
| 220 | CR³ | CR⁴ | H | cyanomethyl | H | H | H | H | H | Br | H | cyclobutyl | 2.12[b] |
| 221 | CR³ | CR⁴ | H | H | dimethylcar- | H | H | H | H | CF₃ | H | cyclobutyl | 2.48[b] |
| | | | | | bamoyl | | | | | | | | |
| 222 | CR³ | CR⁴ | H | ethyl | CH₃ | H | H | H | H | Cl | H | cyclobutyl | 2.73[b] |
| 223 | CR³ | CR⁴ | H | H | piperidin-1- | H | H | H | H | CF₃ | H | cyclobutyl | 3.31[b] |
| | | | | | ylcarbonyl | | | | | | | | |
| 224 | CR³ | CR⁴ | H | CH₃ | H | H | H | H | H | Br | H | cyclobutyl | 2.55[b] |
| 225 | CR³ | CR⁴ | H | propan-2-yl | H | H | H | H | H | CF₃ | H | cyclobutyl | 4.61[b] |
| 226 | CR³ | CR⁴ | H | cyanomethyl | H | H | H | H | H | CF₃ | H | cyclobutyl | 3.01[b] |
| 227 | CR³ | CR⁴ | H | acetyl | H | H | H | H | H | CF₃ | H | cyclobutyl | 3.24[b] |
| 228 | CR³ | CR⁴ | H | ethylsulphanyl | H | H | H | H | H | Br | H | cyclobutyl | 3.37[b] |
| 229 | CR³ | CR⁴ | H | H | H | H | H | H | H | Br | H | cyclobutyl | 2.18[b] |
| 230 | CR³ | CR⁴ | H | H | iodine | H | H | H | H | Cl | H | cyclobutyl | 3.49[b] |
| 231 | CR³ | CR⁴ | H | CH₃ | H | H | H | H | H | Cl | H | cyclobutyl | 2.33[b] |
| 232 | CR³ | CR⁴ | H | H | H | H | H | H | H | CF₃ | H | 1- | 3.36[b] |
| | | | | | | | | | | | | methylcyclo | |
| | | | | | | | | | | | | butyl | |
| 233 | CR³ | CR⁴ | H | H | cyanomethyl | H | H | H | H | Cl | H | cyclobutyl | 1.88[b] |
| 234 | CR³ | CR⁴ | H | H | 1-cyanoethyl | H | H | H | H | Cl | H | cyclobutyl | 3.45[c] |
| 235 | CR³ | CR⁴ | H | ethylsulphanyl | H | H | H | H | H | CF₃ | H | cyclobutyl | 4.8[c] |
| 236 | CR³ | CR⁴ | H | H | H | H | H | H | H | iodine | H | cyclobutyl | 2.19[a] |
| 237 | CR³ | CR⁴ | H | 3-methyl-2- | H | H | H | H | H | Cl | H | cyclobutyl | 1.66[b] |
| | | | | oxoimida- | | | | | | | | | |
| | | | | zolidin-1-yl | | | | | | | | | |
| 238 | CR³ | CR⁴ | H | methoxy | H | H | H | H | H | CF₃ | H | cyclobutyl | 3.48[b] |
| 239 | CR³ | CR⁴ | H | H | prop-2-en-1- | H | H | H | H | CF₃ | H | cyclobutyl | 2.79[b] |
| | | | | | ylcarbamoyl | | | | | | | | |
| 240 | CR³ | CR⁴ | H | H | dimethylcar- | H | H | H | H | Cl | H | cyclobutyl | 1.6[b] |
| | | | | | bamoyl | | | | | | | | |
| 241 | CR³ | CR⁴ | H | H | prop-2-en-1- | H | H | H | H | Cl | H | cyclobutyl | 1.91[b] |
| | | | | | ylcarbamoyl | | | | | | | | |
| 242 | CR³ | CR⁴ | H | cyanomethyl | H | H | H | H | H | Cl | H | cyclobutyl | 1.97[b] |
| 243 | CR³ | CR⁴ | H | propan-2-yl | H | H | H | H | H | Cl | H | cyclobutyl | 3.1[b] |
| 244 | CR³ | CR⁴ | H | H | 2-oxopropyl | H | H | H | H | Cl | H | cyclobutyl | 1.75[b] |
| 245 | CR³ | CR⁴ | H | H | H | H | H | H | H | Cl | CH₃ | cyclobutyl | 3.32[a] |
| 246 | CR³ | CR⁴ | H | H | ethyl(methyl) | H | H | H | H | Cl | H | cyclobutyl | 1.85[b] |
| | | | | | carbammoyl | | | | | | | | |
| 247 | CR³ | CR⁴ | H | dimethylcar- | H | H | H | H | H | Cl | H | cyclobutyl | 1.57[b] |
| | | | | bamoyl | | | | | | | | | |
| 248 | CR³ | CR⁴ | H | H | propan-2-yl- | H | H | H | H | Cl | H | cyclobutyl | 2.68[c] |
| | | | | | carbamoyl | | | | | | | | |
| 249 | CR³ | CR⁴ | H | H | morpholin-4- | H | H | H | H | Cl | H | cyclobutyl | 1.24[b] |
| | | | | | ylcarbonyl | | | | | | | | |
| 250 | CR³ | CR⁴ | H | H | diethylcar- | H | H | H | H | Cl | H | cyclobutyl | 3.19[c] |
| | | | | | bamoyl | | | | | | | | |
| 251 | CR³ | CR⁴ | H | ethyl | H | H | H | H | H | Cl | H | cyclobutyl | 2.67[b] |
| 252 | CR³ | CR⁴ | H | diethylcar- | H | H | H | H | H | Br | H | cyclobutyl | 2.26[b] |
| | | | | bamoyl | | | | | | | | | |
| 253 | CR³ | CR⁴ | H | 3-oxobutyl | H | H | H | H | H | Br | H | cyclobutyl | 2.04[b] |
| 254 | CR³ | CR⁴ | H | tert-butyl | H | H | H | H | H | Br | H | cyclobutyl | 3.67[b] |
| 255 | CR³ | CR⁴ | H | tert-butyl | H | H | H | H | H | Cl | H | cyclobutyl | 3.26[b] |
| 256 | CR³ | CR⁴ | H | 3-oxobutyl | H | H | H | H | H | Cl | H | cyclobutyl | 1.98[b] |
| 257 | CR³ | CR⁴ | H | 1,1-dioxido- | H | H | H | H | H | Cl | H | cyclobutyl | 1.74[b] |
| | | | | isothiazolidin- | | | | | | | | | |
| | | | | 2-yl | | | | | | | | | |
| 258 | CR³ | CR⁴ | H | 1,1-dioxido- | H | H | H | H | H | Br | H | cyclobutyl | 2.25[b] |
| | | | | isothiazolidin- | | | | | | | | | |
| | | | | 2-yl | | | | | | | | | |
| 259 | CR³ | CR⁴ | H | tert-butyl | H | H | H | H | H | CF₃ | H | cyclobutyl | 4.98[b] |
| 260 | CR³ | CR⁴ | H | methylamino | H | H | H | H | H | Cl | H | cyclobutyl | 1.49[b] |
| 261 | CR³ | CR⁴ | H | acetyl(methyl) | H | H | H | H | H | Cl | H | cyclobutyl | 2.49[c] |
| | | | | amino | | | | | | | | | |
| 262 | CR³ | CR⁴ | H | morpholin-4- | H | H | H | H | H | Cl | H | cyclobutyl | 2.39[c] |
| | | | | yl-carbonyl | | | | | | | | | |
| 263 | CR³ | CR⁴ | H | methylsul- | H | H | H | H | H | Cl | H | cyclobutyl | 2.63[c] |
| | | | | phonyl | | | | | | | | | |
| 264 | CR³ | CR⁴ | H | methylsul- | H | H | H | H | H | CF₃ | H | cyclobutyl | 2.76[b] |
| | | | | phonyl | | | | | | | | | |
| 265 | CR³ | CR⁴ | H | methylsul- | H | H | H | H | H | Br | H | cyclobutyl | 2.07[b] |
| | | | | phonyl | | | | | | | | | |

where X¹ = N or CR³
X² = N OR CR⁴

Analogously to the methods given above, it is also possible to obtain the compounds of the formula I, (Ia), (Ib), (Ic) listed in Table 2 below

I, (Ia), (Ib), (Ic)

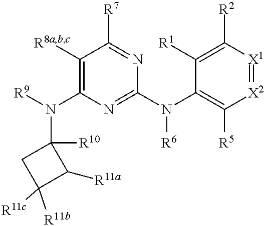

| Ex No | X¹ | X² | R¹ | R² | R³ | R⁴ | R⁵ | R⁶ | R⁷ | R^{8a,8b,8c} | R⁹ | 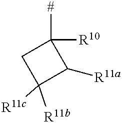 | log p | Y-H |
|---|---|---|---|---|---|---|---|---|---|---|---|---|---|---|
| 2-1 | CR³ | CR⁴ | H | Cl | H | H | H | H | H | Cl | H | cyclobutyl | 3.09^{[a]} | HCl |
| 2-2 | CR³ | CR⁴ | H | H | H | H | H | H | H | Cl | H | cyclobutyl | 1.97^{[a]} | HCl |
| 2-3 | CR³ | CR⁴ | H | OMe | H | OMe | H | H | H | Cl | H | cyclobutyl | 2.23^{[a]} | HCl |
| 2-4 | CR³ | CR⁴ | H | acetyl | H | H | H | H | H | Cl | H | cyclobutyl | 2.04^{[a]} | HCl |
| 2-5 | CR³ | CR⁴ | H | H | isopropyl | H | H | H | H | Cl | H | cyclobutyl | 2.74^{[a]} | HCl |
| 2-6 | CR³ | CR⁴ | H | H | isopropoxy | H | H | H | H | Cl | H | cyclobutyl | 2.21^{[a]} | HCl |
| 2-7 | CR³ | CR⁴ | H | H | (dimethyl- | H | H | H | H | Cl | H | cyclobutyl | 2.58^{[a]} | HCl |
| | | | | | amino)sulphonyl | | | | | | | | | |
| 2-8 | CR³ | CR⁴ | H | H | H | H | H | H | CH₃ | Cl | H | cyclobutyl | 1.95^{[a]} | 4-methylben- |
| | | | | | | | | | | | | | | zenesulphonic acid |

The logP values are measured in accordance with EEC Directive 79/831 Annex V.A8 by HPLC (high performance liquid chromatography) on reserved-phase columns (C 18), using the methods below:
^{[a]}The determination in the acidic range is carried out at pH 2.3 using the mobile phases 0.1% aqueous phosphoric acid and acetonitrile linear gradient from 10% acetonitrile to 95% acetonitrile.
^{[b]}The determination by LC-MS in the acidic range is carried out at pH 2.7 using the mobile phases 0.1% aqueous formic acid and acetonitrile (with 0.1% formic acid) linear gradient from 10% aceetonitrile to 95% acetonitrile.
^{[c]}The determination by LC-MS in the neutral range is carried out at pH 7.8 using the mobile phases 0.001 molar aqueous ammonium bicarbonate solution and acetonitrile linear gradient from 10% acetonitrile to 95% acetonitrile.
Calibration is carried out using straight-chain alkan-2-ones (having 3 to 16 carbon atoms) with known logP values (determination of the logP values by the retention times using linear interpolation between two successive alkanones). The lambda-max values were determined in the maximum of the chromatographic signals using the UV spectra from 200 nm to 400 nm.

### Use Examples

### Example A

### Venturia Test (Apple)/Protective

Solvents: 24.5 parts by weight of acetone
24.5 parts by weight of dimethylacetamide
   Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention Nos. 5, 17, 32, 38, 39, 41, 42, 45, 46, 48, 49, 50, 55, 56, 65, 84, 88, 94, 104, 106, 111, 143, 154, 164, 176, 185, 196, 206, 210, 212, 214, 218, 220, 223, 226, 227, 229, 233, 234, 238, 239, 244, 252, 253, 256, 257, 258, 261, 265, 2-2, 2-3, 2-4, 2-5 from Tables 1 and 2 show, at an active compound concentration of 100 ppm, an efficacy of 70% or more.

### Example B

### Uromyces Test (Bean)/Protective

Solvents: 24.5 parts by weight of acetone
24.5 parts by weight of dimethylacetamide
   Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity young plants are sprayed with the active compound preparation at the stated application rate. After the spray coating has dried on, the plants are inoculated with an aqueous spore suspension of the bean rust pathogen Uromyces appendiculatus and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention Nos. 32, 39, 42, 48, 55, 65, 78, 86, 87, 88, 94, 96, 100, 106, 109, 119, 128, 157, 214, 218, 240, 252, 253, 257, 258, 2-2, 2-4, 2-5 from Tables 1 and 2 show, at an active compound concentration of 100 ppm, an efficacy of 70% or more.

### Example C

### Alternaria Test (Tomato)/Protective

Solvent: 49 parts by weight of N,N-dimethylfoiniamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the active compound preparation at the stated application rate. 1 day after the treatment, the plants are inoculated with a spore suspension of Alternaria solani and then remain at 100% rel. humidity and 20° C. for 24 h. The plants then remain at 96% rel. atmospheric humidity and a temperature of 20° C.

Evaluation is carried out 7 days after the inoculation. 0% means an efficacy which corresponds to that of the control, whereas an efficacy of 100% means that no infection is observed.

In this test, the compounds according to the invention Nos. 4, 5, 16, 32, 40, 46, 50, 51, 52, 55, 56, 65, 88, 90, 93, 94, 96, 97, 100, 102, 104, 110, 116, 117, 118, 119, 120, 125, 135, 137, 138, 141, 146, 152, 153, 155, 157, 165, 176, 178, 188, 190, 196, 200, 201, 203, 205, 206, 208, 212, 214, 218, 223, 224, 227, 231, 234, 239, 240, 241, 243, 244, 247, 251, 253, 256, 257, 258, 260, 261, 264, 265, 2-2, 2-4, 2-5 from Tables 1 and 2 show, at an active compound concentration of 500 ppm, an efficacy of 70% or more.

### Example D

### Sphaerotheca Test (Cucumber)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. The plants are inoculated 1 day after the treatment with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the compounds according to the invention Nos. 1, 5, 19, 31, 32, 35, 36, 38, 39, 41, 42, 44, 45, 46, 47, 48, 49, 51, 54, 55, 57, 58, 59, 60, 61, 62, 63, 64, 65, 73, 75, 76, 77, 78, 79, 82, 83, 84, 88, 94, 100, 104, 109, 110, 128, 129, 138, 143, 146, 148, 149, 155, 156, 157, 164, 175, 176, 185, 188, 205, 206, 208, 210, 212, 215, 216, 222, 223, 224, 225, 226, 227, 229, 231, 233, 237, 240, 242, 243, 244, 246, 247, 249, 251, 252, 254, 256, 257, 258, 259, 261, 263, 264, 2-2, 2-3, 2-5 from Tables 1 and 2 show, at an active compound concentration of 500 ppm, an efficacy of 70% or more.

### Example E

### Puccinia Test (Wheat)/Protective

Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Puccinia recondita. The plants remain in an incubation cabin at 20° C. and 100% relative atmospheric humidity for 48 hours. The plants are then placed in a greenhouse at a temperature of about 20° C. and a relative atmospheric humidity of 80% to promote the development of rust pustules.

Evaluation is carried out 10 days after the inoculation. 0% means an efficacy which corresponds to that of the control, whereas an efficacy of 100% means that no infection is observed.

In this test, the following compounds according to the invention Nos. 1, 2, 3, 4, 5, 6, 10, 12, 13, 15, 16, 17, 18, 19, 23, 32, 33, 36, 38, 39, 40, 41, 42, 44, 45, 46, 48, 50, 51, 53, 54, 55, 56, 57, 58, 59, 60, 61, 62, 63, 64, 65, 66, 68, 70, 71, 72, 73, 74, 75, 77, 78, 79, 80, 81, 82, 83, 84, 85, 86, 87, 88, 89, 90, 91, 92, 93, 94, 95, 96, 97, 98, 99, 100, 101, 102, 104, 105, 106, 108, 117, 119, 123, 128, 129, 138, 139, 141, 143, 146, 149, 152, 153, 154, 155, 156, 157, 160, 162, 164, 166, 167, 171, 172, 173, 174, 175, 176, 177, 178, 179, 180, 181, 184, 185, 186, 187, 188, 190, 191, 192, 195, 196, 197, 200, 201, 202, 203, 204, 205, 206, 209, 210, 211, 212, 214, 215, 216, 217, 218, 220, 221, 223, 225, 226, 227, 228, 229, 231, 233, 234, 237, 239, 240, 241, 242, 246, 247, 248, 249, 250, 252, 253, 257, 258, 259, 262, 263, 264, 265, 2-5 from Tables 1 and 2 show, at an active compound concentration of 1000 ppm, an efficacy of 70% or more.

### Example F

### Fusarium nivale (var. majus) Test (Wheat)/Protective

Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Fusarium nivale (var. majus).

The plants are placed in a greenhouse under translucent incubation hoods at a temperature of about 15° C. and a relative atmospheric humidity of about 100%.

Evaluation is carried out 6 days after the inoculation. 0% means an efficacy which corresponds to that of the control, whereas an efficacy of 100% means that no infection is observed.

In this test, the compounds according to the invention Nos. 23, 31, 36, 38, 39, 40, 42, 45, 48, 49, 55, 57, 59, 83, 94, 96, 109, 110, 111, 116, 123, 125, 126, 135, 137, 140, 148, 149, 150, 152, 154, 155, 157, 164, 166, 167, 175, 176, 188, 209, 214, 215, 218, 220, 224, 226, 240, 242, 2-2, 2-3, 2-5, 2-6 from Tables 1 and 2 show, at an active compound concentration of 1000 ppm, an efficacy of 70% or more.

### Example G

### Leptosphaeria nodorum Test (Wheat)/Protective

Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a spore suspension of Leptosphaeria nodorum. The plants remain in an incubation cabin at 20° C. and 100% relative atmospheric humidity for 48 hours.

The plants are placed in a greenhouse at a temperature of about 20° C. and a relative atmospheric humidity of 80%.

Evaluation is carried out 10 days after the inoculation. 0% means an efficacy which corresponds to that of the control, whereas an efficacy of 100% means that no infection is observed.

In this test, the following compounds according to the invention Nos. 23, 31, 35, 36, 38, 39, 40, 42, 45, 48, 49, 55, 57, 59, 94, 109, 135, 140, 147, 148, 150, 152, 154, 157, 164, 166, 167, 175, 176, 188, 209, 214, 215, 218, 220, 224, 226, 227, 240, 242, 2-2, 2-3, 2-4 from Tables 1 and 2 show, at an active compound concentration of 1000 ppm, an efficacy of 70% or more.

### Example H

### Pyricularia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with an aqueous spore suspension of Pyricularia oryzae. The plants are then placed in a greenhouse at 100% relative atmospheric humidity and 25° C.

Evaluation is carried out 7 days after the inoculation. 0% means an efficacy which corresponds to that of the control, whereas an efficacy of 100% means that no infection is observed.

In this test, the compounds according to the invention Nos. 5, 21, 61, 109, 129, 146, 150, 152, 154, 155, 156, 157, 178, 185, 188, 191, 196, 206, 210, 215, 217, 218, 219, 220, 223, 224, 225, 226, 229, 231, 234, 235, 236, 237, 238, 240, 242, 243, 244, 252, 253, 257, 258, 260, 2-2 from Tables 1 and 2 show, at an active compound concentration of 250 ppm, an efficacy of 80% or more.

### Example I

### Rhizoctonia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with hyphae of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the compounds according to the invention Nos. 5, 39, 41, 46, 48, 50, 55, 57, 61, 94, 100, 109, 119, 123, 125, 128, 129, 138, 139, 143, 146, 149, 150, 152, 154, 155, 156, 157, 185, 188, 191, 196, 206, 210, 215, 217, 220, 223, 224, 225, 226, 229, 234, 235, 236, 237, 238, 240, 243, 244, 252, 253, 257, 258, 260, 2-2 from Tables 1 and 2 show, at an active compound concentration of 250 ppm, an efficacy of 80% or more.

### Example J

### Cochliobolus Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with an aqueous spore suspension of Cochliobolus miyabeanus. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the compounds according to the invention Nos. 39, 41, 138, 148, 152, 156, 185, 191, 196, 210, 223, 226, 229, 235, 240, 242, 243, 244, 253, 257, 258, 260 from Table 1 show, at an active compound concentration of 250 ppm, an efficacy of 80% or more.

### Example K

### Gibberella Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with an aqueous spore suspension of Gibberella zeae. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the compounds according to the invention Nos. 5, 50, 57, 100, 128, 129, 146, 152, 154, 155, 188 and 210 from Table 1 show, at an active compound concentration of 250 ppm, an efficacy of 80% or more:

### Example L

### Meloidogyne Test (Spray Treatment)

Solvent: 80 parts by weight of acetone

To produce a suitable preparation of active compound, 1 part by weight of active compound is mixed with the stated amount of solvent, and the concentrate is diluted with emulsifier-containing water to the desired concentration.

Containers are filled with sand, active compound solution, Meloidogyne incognita egg/larvae suspension and lettuce seeds. The lettuce seeds germinate and the plants develop. On the roots, galls are formed.

After the desired period of time, the nematicidal activity is determined by the formation of galls in %. 100% means that no galls were found; 0% means that the number of galls on the treated plants corresponds to that of the untreated control.

In this test, for example, the following compound of the Preparation Examples shows, at a concentration of 20 ppm, an efficacy of >80%:

### Example NO. 118

### Example M

### Spodoptera frugiperda Test (SPDFR Spray Application)

Solvents: 78.0 parts by weight of acetone
1.5 parts by weight of dimethylformamide
   Emulsifier: 0.6 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Maize plants (Zea mays) are watered with an active compound preparation of the desired concentration and infected with Spodoptera frugiperda larvae.

After the desired period of time, the kill in % is determined. 100% means that all larvae have been killed; 0% means that none of the larvae have been killed.

In this test, for example, the following compound of the Preparation Examples shows, at an application rate of 500 g/ha, a good efficacy of ≧80%:

### Example NO. 221

### Example N

### Boophilus microplus Test (BOOPMI Injection)

Solvent: dimethylsulphoxide

To produce a suitable preparation of active compound, 1 part by weight of active compound is mixed with the stated amount of solvent, and the concentrate is diluted with water to the desired concentration.

The solution of active compound is injected into the abdomen (Boophilus microplus), and the animals are transferred into dishes and kept in a temperature-controlled room. The activity is assessed by examination for deposition of fertile eggs.

After the desired period of time, the effect in % is determined. 100% means that no tick has laid any fertile eggs.

In this test, for example, the following compounds of the Preparation Examples show an activity of ≧80% at an application rate of 20 μg/animal:

### Examples Nos. 83, 88, 100, 129

### Example O

### 1. Herbicidal Pre-Emergence Action (═Pe)

### Test Description

Seeds of monocotyledonous and dicotyledonous harmful plants are placed in sandy loam and covered with soil. The compounds according to the invention, formulated as emulsion concentrates (EC), are then, as an aqueous emulsion with a water application rate of 800 l/ha (converted), applied to the surface of the covering soil.

After the treatment, the pots are placed in a greenhouse and kept under good growth conditions for the test plants. The visual assessment of the damage on the test plants is carried out after a trial period of 2 weeks by comparison with untreated controls (herbicidal effect in percent (%): 100% effect=the plants have died, 0% effect=like control plants). CAPBP=Capsella bursa-pastoris (shepherd's purse), STEME=Stellaria media (common chickweed)

| | | |
|---|---|---|
| Ex. | Test | Test object |

| No. | type | Dose | Unit | CAPBP | STEME |
|---|---|---|---|---|---|
| 88 | PE | 1000 | g/ha | 90 | 80 |
| 156 | PE | 1000 | g/ha | 70 | 70 |
| 142 | PE | 1000 | g/ha | 80 | 70 |

### 2. Herbicidal Post-Emergence Action (═PO)

### Test Description

Seeds of monocotyledonous and dicotyledonous harmful plants are placed in sandy loam, covered with soil and cultivated in a greenhouse under good growth conditions. About 2 weeks after sowing, the test plants are treated at the one-leaf stage. To this end, the compounds according to the invention, formulated as emulsion concentrates (EC), as an aqueous emulsion with a water application rate of 800 l/ha (converted), are sprayed onto the green parts of the plants. After the test plants have been kept in the greenhouse under optimum growth conditions for about 2 weeks, the effect of the preparations is rated visually in comparison to untreated controls (herbicidal effect in percent (%): 100% effect=the plants have died, 0% effect=like control plants). CAPBP=Capsella bursa-pastoris (shepherd's purse), STEME=Stellaria media (common chickweed), ABUTH=Abutilon theophrasti (velvetleaf), AMARE=Amaranthus retroflexus (redroot pigweed)

| | Test | | | Test object | | | |
|---|---|---|---|---|---|---|---|
| Ex. No. | type | Dose | Unit | ABUTH | AMARE | CAPBP | STEME |
| 38 | PO | 1000 | g/ha | 70 | 90 | | |
| 96 | PO | 1000 | g/ha | | 70 | 90 | 70 |
| 100 | PO | 1000 | g/ha | | 70 | 70 | 70 |
| 5 | PO | 1000 | g/ha | | | 70 | 70 |
| 87 | PO | 1000 | g/ha | | 80 | | 70 |
| 88 | PO | 1000 | g/ha | | | 70 | 80 |
| 148 | PO | 1000 | g/ha | 70 | 90 | 80 | 70 |
| 149 | PO | 1000 | g/ha | | 90 | 90 | 70 |
| 155 | PO | 1000 | g/ha | | 70 | 80 | 80 |
| 44 | PO | 1000 | g/ha | | | 80 | 70 |
| 45 | PO | 1000 | g/ha | | 70 | 70 | 80 |
| 50 | PO | 1000 | g/ha | | 70 | | 80 |
| 59 | PO | 1000 | g/ha | 80 | | 70 | 80 |
| 48 | PO | 1000 | g/ha | | 70 | 70 | |
| 120 | PO | 1000 | g/ha | | 90 | 70 | |
| 123 | PO | 1000 | g/ha | | 100 | 80 | 70 |
| 128 | PO | 1000 | g/ha | | 90 | 80 | 90 |
| 129 | PO | 1000 | g/ha | | | 70 | 70 |
| 142 | PO | 1000 | g/ha | 70 | 80 | | |

1. A method of protecting crops from animal pests and/or phytopathogenic fungi and/or weeds comprising applying to an animal pest and/or a habitat thereof, and/or to phytopathogenic fungi and/or a habitat thereof, and/or to a weed and/or a habitat thereof, a compound of formula (I): 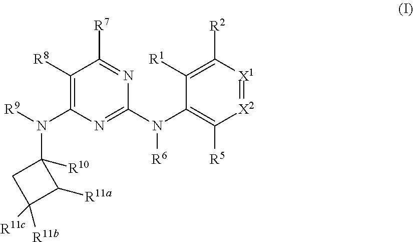
in which the symbols have the following meanings:
X¹ is nitrogen or CR³
X² is nitrogen or CR⁴
and where X¹ and X² are not both nitrogen
R¹ and R⁵ independently of one another are hydrogen, C₁-C₄-alkyl, C₁-C₄-alkoxy or Hal
R² to R⁴ independently of one another are hydrogen, halogen, cyano, nitro, a 3- to 8-membered unsubstituted or substituted, saturated or unsaturated cycle which may contain no or up to four heteroatoms selected from the group consisting of N, O and S, where any two oxygen atoms are not adjacent to one another, OR¹², O(CH₂)ₘOR¹², O(CH₂)ₘN(R¹²)₂, O[C(R¹²)₂]ₘOR¹², O[C(R¹²)₂], N(R¹²)₂, OSO₂N(R¹²)₂, OCON(R¹²)₂, OCOR¹³, SF₅, SR¹², SOR¹², SO₂R¹², SON(R¹²)₂, SO₂N(R¹²)₂, C═OR¹², CH═NOR¹², CR¹³═NOR¹², COCl, CON(R¹²)₂, COOR¹², COO(CH₂)ₘOR¹², CO(CH₂)ₘCN, NR¹²COR¹², NR¹²COOR¹³, NR¹²(C═S)OR¹³, N(R¹²)₂, NR¹²SO₂R¹³, NR¹²SOR¹³, C(R¹²)₂OR¹², [C(R¹²)₂]ₘCN, (CH₂)ₘC(R¹²)₂OR¹², (CH₂)ₘOR¹², (CH₂)ₘSR¹², [C(R¹²)₂]ₘSR¹², (CH₂)ₘSOR¹², (CH₂)ₘSO₂R¹², (CH₂)ₘSON(R¹²)₂, (CH₂)ₘSO₂N(R¹²)₂, (CH₂)ₘN(R¹²)₂, [C(R¹²)₂]ₘN(R¹²)₂, (CH₂)ₘCOOR¹², (CH₂)ₘCOR¹², [C(R¹²)₂]ₘOR¹², [C(R¹²)₂]ₘCOR¹², [C(R¹²)₂]ₘCON(R¹²)₂, (CH₂)ₘNR¹²COR¹², (CH₂)ₘNR¹²COOR [C(R¹²)₂]ₘNR¹²COR¹², [C(R¹²)₂]ₘNR¹²COOR¹³, [C(R¹²)₂]ₘNR¹²OR¹², unsubstituted or substituted C₁-C₈-alkyl, C₂-C₆-alkenyl, C₁-C₈-haloalkyl; where m=1−4 where additionally or independently thereof two adjacent radicals R², R³ or R⁴, if appropriate via R¹² or R¹³, may form a 3- to 7-membered unsubstituted or substituted, saturated or unsaturated cycle which may contain no or up to four heteroatoms selected from the group consisting of N, O and S, where any two oxygen atoms are not adjacent to one another,
where the substituents independently of one another are selected from the group consisting of hydrogen, fluorine, chlorine or bromine, C₁-C₄-alkyl, C₁-C₄-alkoxy, hydroxyl, oxo, C₁-C₄-haloalkyl and cyano,
R⁶ is hydrogen, C₁-C₂-alkyl, C₁-C₄-alkoxy(C₁-C₄)alkyl, C₁-C₄-trialkylsilyl, C₁-C₄-trialkylsilylethyl, C₁-C₄-dialkylmonophenylsilyl, formyl, (C₁-C₄-alkyl)carbonyl, (C₁-C₄-alkoxy-C₁-C₄-alkyl)carbonyl, (C₃-C₆-alkenyloxy)carb (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulphinyl or C₁-C₂-alkylsulphonyl,
where the substituents on said benzyl, C₂-C₆-alkenyl, or C₂-C₆-alkynyl, independently of one another are selected from the group consisting of hydrogen, fluorine, chlorine or bromine, C₁-C₄-alkyl, C₁-C₄-alkoxy, hydroxyl, C₁-C₄-haloalkyl and cyano,
R⁷ is hydrogen, C₁-C₃-alkyl, cyano or C₁-C₃-haloalkyl,
R⁸ is chlorine, bromine, iodine, cyano, methyl, CF₃, CCl₃, CFH₂ or CF₂H,
R⁹ is hydrogen, C₁-C₂-alkyl, C₁-C₄-alkoxy(C₁-C₄)alkyl, C₁-C₆-trialkylsilyl, C₁-C₄-tri alkyl silyl ethyl, C₁-C₄-di alkylmonophenylsilyl, (C₁-C₄-alkyl)carbonyl, (C₁-C₄-haloalkyl)carbonyl, (C₁-C₄-alkoxy)carbonyl, unsubstituted or substituted benzyl, unsubstituted or substituted C₂-C₆-alkenyl, unsubstituted or substituted C₂-C₆-alkynyl, C₁-C₆-alkylsulphinyl, C₁-C₆-alkylsulphonyl, C₁-C₆-haloalkylsulphinyl or C₁-C₆-haloalkylsulphonyl,
where the substituents on said benzyl, C₂-C₆-alkenyl, or C₂-C₆-alkynyl independently of one another are selected from the group consisting of fluorine, chlorine and/or bromine atoms, cyano, hydroxyl, methoxy, CF₃
R¹⁰ is hydrogen, methyl, trifluoromethyl, nitrile, phenyl, meta-chlorophenyl, meta-fluorophenyl, para-chlorophenyl, para-fluorophenyl, benzyl, meta-chlorobenzyl, COOH, COOMe or COOEt,
R^{11a} is hydrogen, phenyl, para-methoxyphenyl or COOMe,
R^{11b} is hydrogen, fluorine, phenyl, para-methoxyphenyl, COOH or COOEt,
R^{11c} is hydrogen or fluorine,
where in each case only one of the radicals R¹⁰, R^{11a}, R^{11b} or R^{11c} is not a hydrogen,
or
R^{11b} and R^{11c} both represent fluorine,
R¹² are identical or different and are hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another
or
if two radicals R¹² are attached to a nitrogen atom, two radicals R¹² may form a 3-to 7-membered unsubstituted or substituted, saturated or unsaturated cycle which may contain up to four further heteroatoms selected from the group consisting of N, O and S, where any two oxygen atoms are not adjacent to one another,
or
if two radicals R¹² are adjacent in a grouping NR¹²COR¹² NR¹²SOR¹² NR¹²SO₂R¹² NR¹²SO₂NR¹² or NR¹²SO₂NR¹², the two adjacent radicals R¹² may form a 3- to 7-membered unsubstituted or substituted, saturated or unsaturated cycle which may contain up to four further heteroatoms selected from the group consisting of N, O and S, where any two oxygen atoms are not adjacent to one another,
R¹³ are identical or different and are C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another,
where two R¹³ may form a 3- to 7-membered unsubstituted or substituted, saturated or unsaturated cycle which may contain up to four further heteroatoms selected from the group consisting of N, O and S, where any two oxygen atoms are not adjacent to one another
and where possible substituents are selected from the following list:
fluorine, chlorine, bromine, iodine, cyano, nitro, CF₃, CFH₂, CF₂H, C₂F₅, CCl₃, hydroxyl, OMe, OEt, OPr, OisoPr, OBu, OsecBu, OisoBu, OtertBu, O(CH₂)₂OCH₃, O(CH₂)₃OCH₃, O-cyclopentyl, O-phenyl, OCF₃, OCF₂H, OCF₂CF₃, OCF₂CF₂H, SH, SMe, SEt, SCF₃, SCF₂H, SPh, SCF₅, SO₂Me, SO₂CF₃, SOMe, SOEt, CO₂H, CO₂CH₃, CO₂Et, CO₂Pr, CO₂ isoPr, CO₂tertBu, COMe, COCF₃, NH₂, NHMe, NMe₂, NHEt, NEt₂, NHPr, NHisoPr, NHnBu, NHtertBu, NHisoBu, NHsecBu, cyclopropylamino, formyl, CH₂CN, CHMeCN, CH₂COCH₃, CH₂OMe, (CH₂)₂OMe, (CH₂)₃OMe, CH₂OH, CH₂SMe, (CH₂)₂SMe, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, cyclohexylmethyl, neopentyl, prop-2-en-1-yl, 1-methylprop-2-en-1-yl, but-3-en-1-yl, (trimethylsilyl)methyl, cyclopropyl, cyclobutyl, cyclopentyl, cyclohexyl, phenyl, benzyl, —CH₂CH═CH₂, —CH(CH₃)CH═CH₂, —CH₂CE-CH,
or an agrochemically active salt thereof.

2. The method according to claim 1 wherein
X¹ is nitrogen or CR³
X² is nitrogen or CR⁴
and where X¹ and X² are not both nitrogen,
R¹ and R⁵ independently of one another are H, C₁-C₂-alkyl, C₁-C₂-alkoxy, F, Cl or Br
R² to R⁴ independently of one another are hydrogen, fluorine, chlorine, bromine, iodine, cyano, nitro, hydroxyl, O-C₁-C₄-alkyl, O—(C₁-C₃-haloalkyl), O—(C₃-C₆-cycloalkyl), O-C₂-C₄-alkenyl, O-C₂-C₄-alkynyl, O(CH₂)ₘO(C₁-C₄-alkyl), OPh, O(CH₂)ₘN(C₁-C₄-alkyl)₂, O(CH₂)ₘNH(C₁-C₄-alkyl), OCH(C₁-C₄-alkyl)CH₂O(C₁-C₄-alkyl), OSO₂N(C₁-C₄-alkyl)₂, OCONH(C₁-C₄-alkyl), OCON(C₁-C₄-alkyl)₂, OCO(C₁-C₄-alkyl), SF₅, SH, S-C₁-C₄-alkyl, S-C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂CH₂CN, SO₂(C₂-C₄-alkynyl), SONH(C₁-C₄-alkyl), SON(C₁-C₄-alkyl)₂, SO₂NH₂, SO₂NH(C₁-C₄-alkyl), SO₂N(C₁-C₄-alkyl)₂, SO₂NHCO(C₁-C₄-alkyl), SO₂NHPh, SO₂NH(CH₂)ₘN(C₁-C₄-alkyl)₂, SO₂NH(C₂-C₄-alkenyl), (C₁-C₄-alkyl)carbonyl, (C₁-C₃-haloalkyl)carbonyl, CH═NO(C₁-C₄-alkyl), C(C₁-C₄-alkyl)═NO(C₁-C₄-alkyl), CO(CH₂)ₘCN, CONH(C₁-C₄-alkyl), CON(C₁-C₄-alkyl)₂, CONH(C₁-C₃-haloalkyl), CONH(C₂-C₄-alkenyl). CONH(C₂-C₄-alkynyl), CONHCH₂C(═CH₂)CH₃, CONHCH(CH₃)CH₂O(C₁-C₄-alkyl), CONH(CH₂)ₘO(C₁-C₄-alkyl), CONHPh, COCH₂N(C₁-C₄-alkyl)₂, CONH-cyclopropyl, CONH-cyclopropylmethyl, piperidin-1-ylcarbonyl, morpholin-4-ylcarbonyl, (4-methylpiperazin-1-yl)carbonyl, COOH, COCl, (C₁-C₄-alkoxy)carbonyl, CO₂(CH₂)ₘO(C₁-C₄-alkyl), NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCOC((C₁-C₄-alkyl)₂CH₂Hal, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NHCO₂(C₁-C₄-alkyl), NHCO₂Ph, NHCO₂CH₂CH₂Hal, N(C₁-C₄-alkyl)CO₂(C₁-C₄-alkyl), NH(C═S)O(C₁-C₄-alkyl), NH₂, NH(C₁-C₄-alkyl), N(C₁-C₄-alkyl)₂, cyclopropylamino, NHCH(C₁-C₄-alkyl)CH₂O(C₁-C₄-alkyl), acetyl(cyclopropyl)amino, [(1-methylcyclopropyl)carbonyl]amino, morpholin-1-yl, morpholin-4-ylmethyl, NHSO(C₁-C₄-alkyl), NHSO(C₁-C₃-haloalkyl), NHSO₂(C₁-C₄-alkyl), NHSO₂(C₁-C₃-haloalkyl), CH₂CN, CH(C₁-C₄-alkyl)CN, (CH₂)ₘSO₂(C₁-C₄-alkyl), (CH₂)ₘSO₂NH(C₁-C₄-alkyl), (CH₂)ₘCO(C₁-C₄-alkyl), CH(C₁-C₄-alkyl)CO(C₁-C₄-alkyl), (CH₂)ₘCO-cyclopropyl, (CH₂)ₘCO₂(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CHCHF₂OH, CH₂OH, (CH₂)ₘS(C₁-C₄-alkyl), C(CH₃)₂S(C₁-C₄-alkyl), CH₂NHCOO(C₁-C₄-alkyl), CH₂NHCOOBn, CH₂NH(CH₂)ₘO(C₁-C₄-alkyl), (CH₂)ₘN(C₁-C₄-alkyl)₂, (CH₂)ₘNHCO(C₁-C₄-alkyl), (CH₂)ₘNHCO(C₁-C₃-haloalkyl), (CH₂)ₘNH(C₁-C₄-alkyl), (CH₂)ₘN(C₁-C₄-alkyl)₂, CH₂COO(C₁-C₄-alkyl), C₁-C₅-alkyl, C₃-C₆-cycloalkyl, 1-methoxycyclopropyl, 1-chlorocyclopropyl, cyclopenten(1)yl, 2-oxocyclopentyl, cyclohexylmethyl, C₂-C₆-alkenyl, (trimethylsilyl)methyl, C₁-C₃-haloalkyl, 4-(tent-butoxycarbonyl)piperazin-1-yl, morpholin-4-ylsulphonyl, [(4,6-dimethylpyrimidin-2-yl)amino]sulphonyl, 2-oxopyrrolidin-1-yl, 1H-tetrazol-5-yl, 2-oxo-1,3-oxazolidin-3-yl, (cyclopropylcarbonyl)amino, (2-furoyl amino), (3-methyl-2,5-dioxoimidazolidin-1-yl), (piperidin-1-ylethyl)amino, 5-methyl-2-oxo-1,3-oxazolidin-3-yl, cyclopropyl(trifluoroacetyl)amino, (1-methyl cyclopropyl)carbonyl amino, 2,5-dioxopyrrolidin-1-yl, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-methyl-2-oxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, 2-thienyl, piperidin-1-ylsulphonyl, 1,3-thiazol-2-yl, 1,3-thiazol-4-yl, (morpholin-4-ylsulphonyl)methyl, (piperidin-1-ylsulphonyl)methyl, [(4-methylphenyl)amino]sulphonyl, (pyrrolidin-1-ylsulphonyl)methyl, 2-oxoimidazolidin-1-yl, 3-methyl-5-oxo-4,5-dihydro-1H-pyrazol-1-yl, (3,4-dimethyl-5-oxo-4,5-dihydro-1H-pyrazol-1-yl), (1-methylcyclopentyl), pyrrolidin-1-yl, piperidin-1-yl, 2-oxo-2,5-dihydro-1H-pyrrol-1-yl, 3,3-dimethyl-2-oxocyclopentyl, 1-oxo-1,3-dihydro-2H-isoindol-2-yl, 3-oxo-4,5-dimethyl-2,4-dihydropyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-2,3 a,4,5,6,7-hexahydroindazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 2,5-dioxopyrrolidin-1-yl, 3-oxo-4,4-dimethylpyrazolidin-1-yl, 3-oxopyrazolidin-1-yl, 3-oxopyrazolidin-1-yl, (2-oxopyrrolidin-1-yl)methyl, (2-oxopiperidin-1-yl)methyl, 2-oxopiperidin-1-yl, 3-oxomorpholin-4-yl, 2-oxoazetidin-1-yl, 2,5-dioxo-2,5-dihydro-1H-pyrrol-1-yl, 3,5-dimethylpiperidin-1-yl, 4-(tert-butoxycarbonyl)piperazin-1-yl, (4-methylphenyl)sulphamoyl, (3-fluoro-2,2-dimethylpropanoyl)amino, (3-chloro-2,2-dimethylpropanoyl)amino, 5-ethoxy-3,4-dimethyl-1H-pyrazol-1-yl, acetyl(cyclohexyl)amino, 2-furoylamino, cyclopropylcarbamoyl, 2,2,2-(trifluoroethyl)carbamoyl, 5-ethoxy-3-(trifluoromethyl)-1H-pyrazol-1-yl, 3-(2-chloroethyl)-2-oxoimidazolidin-1-yl, 2-oxoazepan-1-yl, 2-oxopyridin-1(2H)-yl, 3-oxobutyl, acetyl(methoxy)amino,1,1-dioxidoisothiazolidin-2-yl, 1,1-dioxidotetrahydrothiophen-2-yl, 5-methyl-1,1-dioxido-1,2,5-thiadiazolidin-2-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 4-methyl-5-oxo-4,5-dihydro-1H-1,2,4-triazol-1-yl, 3-methyl-5-oxo-2,5-dihydro-1H-pyrazol-1-yl, 4-oxo-1,3-oxazolidin-3-yl, 2-(methoxymethyppyrrolidin-1-yl, 2-oxo cyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydro-furan-2-yl, furan-2-yl, 1,3-dioxolan-2-yl, 2-methyl-1,3-dioxolan-2-yl, 1-(methylethyl)-2-oxo-1,3-oxazolidin-3-yl, 1,1-dioxido-1,2-thiazinan-2-yl, 6-methyl-1,1-dioxido-1,2,6-thiadiazinan-2-yl, 3-5-methyl-1,1-dioxido-1,2,5-thiadiazolidin-2-yl, 3-6-methyl-1,1-dioxido-1,2,6-thiadiazinan-2-yl,
where m=1-3
and, if in each case two adjacent radicals R², R³ or R⁴, if appropriate via R¹² or R¹³, form a cycle with a subunit from the general formula (I): 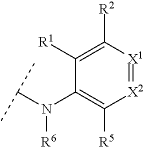
the cycle may be (2-oxo-2,3-dihydro-1H-indol-5-yl)amino, 1H-indol-6-ylamino, 1H-indol-5-ylamino, [2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-o xo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ is hydrogen, C₁-C₂-alkyl, triethylsilyl, trimethylsilyl, tert-butyldimethylsilyl, dimethylphenylsilyl, C₁-C₂-alkoxy(C₁-C₂)alkyl, formyl, (C₁-C₄-alkyl)carbonyl, (methoxymethyl)carbonyl, (allyloxy)carbonyl, (cyclopropyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₄-alkenyl, unsubstituted or substituted C₂-C₄-alkynyl, C₁-C₂-alkylsulphinyl or C₁-C₂-alkylsulphonyl,
where the substituents on said benzyl, C₂-C₄-alkenyl or C₁-C₄-alkynyl independently of one another are selected from the group consisting of hydrogen, fluorine, chlorine or bromine, C₁-C₄-alkyl, C₁-C₄-alkoxy, hydroxyl, C₁-C₄-haloalkyl or cyano,
R⁷ is hydrogen, methyl, CF₃, CFH₂, cyano, or CF₂H
R⁸ is chlorine, bromine, iodine, cyano, methyl, CF₃, CCl₃, CFH₂, or CF₂H
R⁹ is hydrogen, C₁-C₂-alkyl, C₁-C₂-alkoxy(C₁-C₂)alkyl, C₁-C₆-trialkylsilyl, C₁-C₄-trialkylsilylethyl, C₁-C₄-dialkylmonophenylsilyl, (C₁-C₄-alkyl)carbonyl, (C₁-C₄-haloalkyl)carbonyl, (C₁-C₄-alkoxy)carbonyl, benzyl, 4-methoxybenzyl, C₂-C₄-alkenyl, C₂-C₄-alkynyl, C₁-C₄-alkylsulphinyl, C₁-C₄-alkylsulphonyl, C₁-C₄-haloalkylsulphinyl or C₁-C₄-haloalkylsulphonyl,
R¹⁰ is hydrogen, methyl, trifluoromethyl, nitrile, phenyl, meta-chlorophenyl, meta-fluorophenyl, para-chlorophenyl, para-fluorophenyl, benzyl, meta-chlorobenzyl, COOH, COOMe or COOEt,
R^{11a} is hydrogen, phenyl, para-methoxyphenyl or COOMe,
R^{11b} is hydrogen, fluorine, phenyl, para-methoxyphenyl, COOH or COOEt,
R^{11c} is hydrogen or fluorine,
where in each case only one of the radicals R¹⁰, R^{11a}, R^{11b} or R^{11c} is not hydrogen,
or
R^{11b} and R^{11c} both represent fluorine.
R¹² are identical or different and are hydrogen, unsubstituted or substituted C₁-C₆-alkyl, unsubstituted or substituted C₁-C₆-haloalkyl, unsubstituted or substituted C₃-C₆-cycloalkyl, C₁-C₄-tri alkyl silyl, unsubstituted or substituted C₂-C₄-alkenyl, unsubstituted or substituted C₃-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where any two oxygen atoms are not adjacent to one another
or an agrochemically active salt thereof.

3. The method according to claim 1 wherein the compound of formula (I) is applied to the animal pest and/or the habitat thereof and/or to the phytopathogenic harmful fungi and/or the habitat thereof their habitat.

4. A compound of formula (I) which is a compound of formula (Ia) 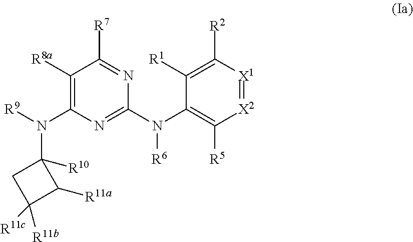
in which the symbols have the following meanings:
R^{8a} represents chlorine, iodine, CFH₂, CF₂H, CCl₃, cyano or Me and
X¹, X², R¹ to R⁷, R^{1-A}, R⁹, R¹⁰, R^{11a}, R^{11b}, R^{11c}, R¹² and R¹³ have the meanings as defined in claim 1,
or an agrochemically active salt thereof.

5. A compound of formula (I) which is a compound of formula (Ib) 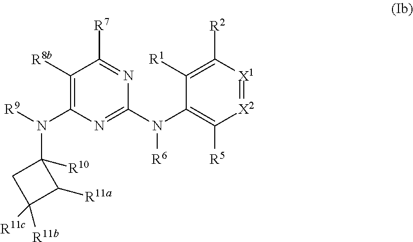
in which the symbols have the following meanings:
R^{8b} represents CF₃ and
X¹, X², R¹ to R⁷, R^{1-A}, R⁹, R¹⁰, R^{11a}, R^{11b} R^{11c} R¹² and R¹³ have the meanings according
as defined in claim 1,
or an agrochemically active salt thereof,
except for those cases wherein a subunit of the general formula (Ib) 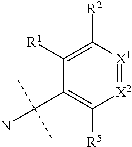
wherein X¹ represents CR³ and X² represents CR⁴ and
either R² and R³ or R³ and R⁴ together with the subunit form a (1H-2-hydroxyindol-6-yl), (1H-2-hydroxyindol-7-yl) or (1H-3,4-dihydro-2-oxoquinolin-7-yl) radical;
or R³ is CON(R¹²)₂ and both radicals R¹² together with the nitrogen atom to which they are attached form a 4-methyl-1,4-piperazinyl radical.

6. A compound of formula (I) which is a compound of formula (Ic) 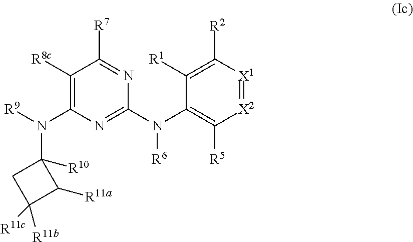
in which the symbols have the following meanings:
R^{8c} represents Br
and
X¹, X², R⁶, R⁷ R^{1-A}, R⁹, R¹⁰, R^{11a}, R^{11b}, R^{11c}, R¹² and R¹³ have the meanings as defined in claim 1, or an agrochemically active salt thereof,
except wherein
(i): X¹ represents CR³ and R² and R³ in a subunit from the general formula (Ic): 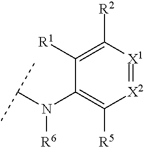
form a (1H-indazol-6-yl)amine,
or
(ii): X¹ represents CR³ and X² represents CR⁴ and R⁴ and R³ in the subunit from the general formula (Ic) form a (1H-indazol-6-yl)amine.

7. (canceled)

8. (canceled)

9. (canceled)

10. (canceled)

11. A process for preparing a compound of formula (Ia), formula (Ib) or formula (Ic) as defined in claim 4, 5, or 6 which process comprises at least one of steps (a) to (e) below:
a) reacting a 2,4-dihalopyrimidine of the formula (III) in the presence of a base, if appropriate in the presence of a solvent, if appropriate in the presence of a catalyst with a cyclobutylamine of formula (II) to give a compound of the formula (V), 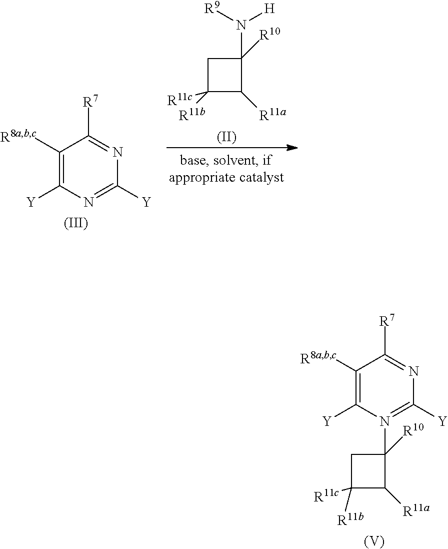
where Y═F, Cl, Br, I;
b) reacting the compound of the formula (V), if appropriate in the presence of an acid, if appropriate in the presence of a solvent, with an aromatic amine of formula (IV), 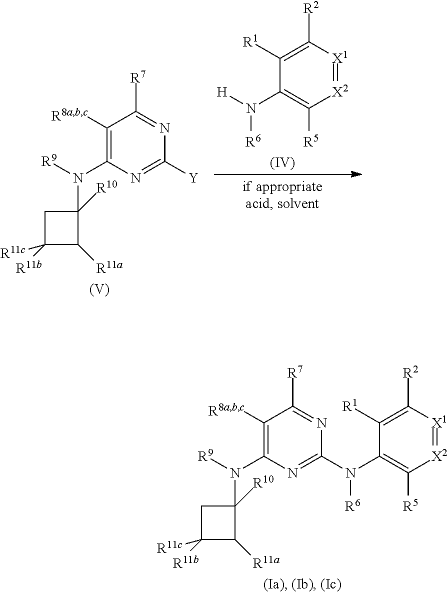
where Y═F, Cl, Br, I;
c) reacting a compound of the formula (VIb), if appropriate in the presence of an acid and in the presence of a solvent, with an aromatic amine of formula (IV) to give a compound of formula (IX), 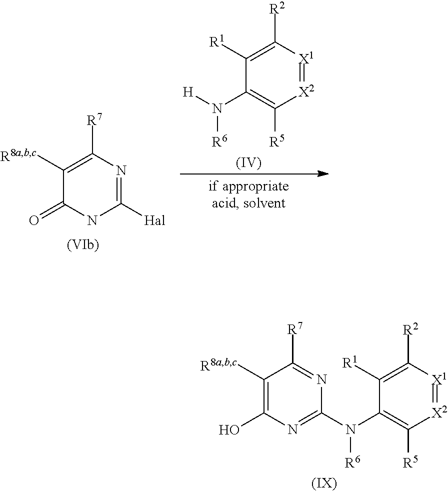
where Hal=F, Cl, Br, I;
d) reacting the compound of formula (IX), if appropriate in the presence of a solvent, with a halogenating agent to give a compound of the formula (X), 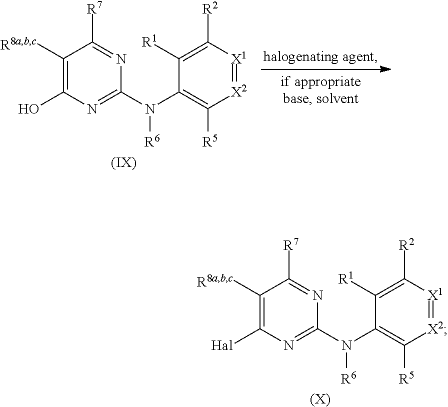
e) reacting the compound of the formula (X) in the presence of a base, if appropriate in the presence of a solvent, if appropriate in the presence of a catalyst with a cyclobutylamine of the formula (II) to give a compound of formula (Ia), formula (Ib), or formula (Ic) 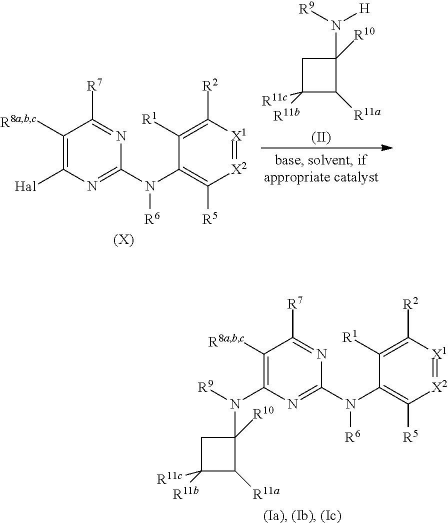
where the definitions of the radicals R¹ to R^{11c} and X¹ and X² are defined as in any one of claims 4, 5, or 6, and Y and Hal represent F, Cl, Br, I.

12. A composition for controlling animal pests and/or phytopathogenic fungi comprising a compound of formula (Ia) according to claim 4 and an extender and/or a surfactant.

13. A process for preparing a composition according to claim 12 comprising mixing the compound of formula (Ia) with a surfactant and/or extender.

14. A method of controlling animal pests and/or phytopathogenic fungi comprising applying to an animal pest and/or a habitat thereof, and/or phytopathogenic fungi and/or a habitat thereof, a compound of formula (Ia) according to claim 4.

15. A composition for controlling animal pests and/or phytopathogenic fungi comprising a compound of formula (Ib) according to claim 5 and an extender and/or a surfactant.

16. A process for preparing a composition according to claim 15 comprising mixing the compound of formula (Ib) with a surfactant and/or extender.

17. A method of controlling animal pests and/or phytopathogenic fungi comprising applying to an animal pest and/or a habitat thereof, and/or phytopathogenic fungi and/or a habitat thereof, a compound of formula (Ib) according to claim 5.

18. A composition for controlling animal pests and/or phytopathogenic fungi comprising a compound of formula (Ic) according to claim 6 and an extender and/or a surfactant.

19. A process for preparing a composition according to claim 18 comprising mixing the compound of formula (Ic) with a surfactant and/or extender.

20. A method of controlling animal pests and/or phytopathogenic fungi comprising applying to an animal pest and/or a habitat thereof, and/or phytopathogenic fungi and/or a habitat thereof, a compound of formula (Ic) according to claim 6.
